(19) United States
(12) Patent Application Publication
(10) Pub. No.: US 2024/0279324 A1
(43) Pub. Date: Aug. 22, 2024

(54) MULTISPECIFIC ANTAGONISTS

(71) Applicant: GENSUN BIOPHARMA INC., NEWBURY PARK, CA (US)

(72) Inventors: Jackie SHENG, Thousand Oaks, CA (US); Tyler LIBAN, Camerillo, CA (US); Gopal SAPPARAPU, Oak Park, CA (US)

(21) Appl. No.: 18/562,994

(22) PCT Filed: Jun. 2, 2022
(86) PCT No.: PCT/US2022/072716

§ 371 Date: Nov. 21, 2023

Related U.S. Application Data
(60) Provisional application No. 63/202,262, filed on Jun. 3, 2021.

(51) Int. Cl. C07K 16/22; A61P 35/00; C07K 16/28

(52) U.S. Cl. CPC C07K 16/22; A61P 35/00; C07K 16/2863; C07K 2317/31

(57) ABSTRACT

Multispecific antagonists for targeting angiogenesis pathways are disclosed. The multispecific antagonists may be used for the treatment of disorders associated with angiogenesis pathways.

This application is a U.S. National Stage Entry of PCT International Application No. PCT/US2022/072716, filed Jun. 2, 2022, which claims priority to U.S. provisional Application No. 63/202,262, filed on Jun. 3, 2021, which is incorporated herein by reference in its entirety.

### FIELD

The present application relates generally to multispecific antagonists capable of modulating angiogenesis pathways and the use of such antagonist for the treatment of disorders associated with angiogenesis pathways.

### BACKGROUND

Age-related macular degeneration (AMD) is the most common cause of uncorrectable severe vision loss in people aged 55 years and older in the developed world. There are two forms of AMD, atrophic or dry AMD and neovascular or wet (exudative) AMD. Typically AMD begins as dry AMD. Dry AMD is characterized by the formation of yellow plaque like deposits called drusen in the macula, between the retinal pigment epithelium (RPE) and the underlying choroid. About 15% of dry AMD patients develop wet AMD; the neovascular form of wet AMD is responsible for most cases of severe vision loss. Indeed, 80% of those with severe visual loss (worse than 20/200 Snellen acuity) have the neovascular form. Choroidal neovascularization (CNV) secondary to AMD accounts for most cases of AMD-related severe vision loss. Once neovascular disease develops in one eye, the risk of developing neovascular disease in the other eye of the same person is approximately 40% by five years.

The integrity of the macula, located in the central retina in the back of the eye, is critical for vision. The incidence of progression from non-neovascular AMD to neovascular AMD is increased by the presence of numerous large and confluent drusen in the macula, as well as by the presence of pigment in the macula. Wet AMD occurs when abnormal growth of blood vessels in the back of the eye damages the macula. Wet AMD causes blurriness, darkness, or distortion in the center of the field of vision, thus reducing the individual's ability to read, drive, and see faces.

VEGF over-expression has been implicated in a variety of angiogenic disorders of the eye. For example, VEGF is known to play an important role in the development and severity of retinopathy of prematurity (ROP) and other ocular neovascular diseases. Given the prominent role of VEGF in angiogenesis generally, and in such eye disorders specifically, a number of therapeutic strategies for inhibiting VEGF activity have been developed. However, current angiogenesis blockade therapies are typically limited to blocking VEGF only. Thus, there is a need for alternative or improved strategies for VEGF blockade in patients suffering from angiogenic disorders, such AMD and various forms of cancer.

### SUMMARY

One aspect of the present application relates to a bispecific antagonist, comprising: an immunoglobulin scaffold comprising CH3 domains; a first targeting domain comprising one or more VEGF or VEGFR binding domains and a second targeting domain comprising one or more Tie2 receptor or Ang binding domains.

In some embodiments, the first targeting domain comprises SEQ ID NO:1. In some embodiments, the first targeting domain comprises SEQ ID NO:88 and SEQ ID NO:89.

In some embodiments, the second targeting domain is inserted into a loop region of the CH3 domain. In some embodiments, the second targeting domain is linked to a N-terminal or a C-terminal of the immunoglobulin scaffold. In some embodiments, the second targeting domain comprises a single copy or two copies of SEQ ID NO:6.

In some embodiments, the first targeting domain comprises SEQ ID NO:1 and is attached to a C-terminal of the immunoglobulin scaffold, and the second targeting domain comprises SEQ ID NO:7 and is inserted into a loop region of the CH3 domain of the immunoglobulin scaffold

In some embodiments, the first targeting domain comprises SEQ ID NO:1 and is attached to a N-terminal of the immunoglobulin scaffold, and wherein the second targeting domain comprises SEQ ID NO:7 and is linked to a C-terminal of the immunoglobulin scaffold.

Another aspect of the present application relates to a trispecific antagonist comprising: (1) an immunoglobulin scaffold comprising a CH3 domain, (2) a first targeting domain comprising one or more VEGF or VEGFR binding domains, (3) a second targeting domain comprising one or more Tie2 receptor or Ang binding domains, and (4) a third targeting domain comprising one or more PD-1 binding domains.

In some embodiments, the first targeting domain comprises SEQ ID NO:88 and SEQ ID NO:89.

In some embodiments, the second targeting domain is inserted into a loop region of the CH3 domain. In some embodiments, the second targeting domain comprises SEQ ID NO:2. In some embodiments the second targeting domain comprises a single copy or two copies of SEQ ID NO:6.

In some embodiments, the third targeting domain comprises SEQ ID NO:90 and SEQ ID NO:91.

In some embodiments, the first targeting domain comprises SEQ ID NO:88 and SEQ ID NO:89 and is attached to a C-terminal of the immunoglobulin scaffold, the second targeting domain comprises SEQ ID NO:2 and is inserted into a loop region of the CH3 domain of the immunoglobulin scaffold, and the third targeting domain comprises SEQ ID NO:90 and SEQ ID NO:91 and is attached to a N-terminal of the immunoglobulin scaffold.

In some embodiments, the first targeting domain comprises SEQ ID NO:88 and SEQ ID NO:89 and is attached to a C-terminal of the immunoglobulin scaffold, the second targeting domain comprises SEQ ID NO:7 and is inserted into a loop region of the CH3 domain of the immunoglobulin scaffold, and the third targeting domain comprises SEQ ID NO:90 and SEQ ID NO:91 and is attached to a N-terminal of the immunoglobulin scaffold.

Another aspect of the present application relates to a pharmaceutical composition comprising the bispecific or trispecific antagonist of the present application and a pharmaceutically acceptable carrier.

Another aspect of the present application relates to a method for treating an angiogenic eye disorder in a subject. The method includes the step of administering to a subject in need thereof an effective amount of a pharmaceutical composition described herein. In certain embodiments, the angiogenic eye disorder is selected from the group consisting of wet macular degeneration, diabetic retinopathy, retinopathy of prematurity, choroidal neovascularization, neovascular glaucoma, corneal neovascularization, and restenosis following glaucoma treatment, angiogenesis resulting from corneal transplantation surgery, and angiogenesis resulting from cataract surgery.

Another aspect of the present application relates to a method for treating cancer in a subject. The method comprises administering the subject an effective amount of the pharmaceutical composition of the present application.

### BRIEF DESCRIPTION OF THE DRAWINGS

FIG. 1 shows exemplary bispecific antagonist configurations containing an Ang2-specific peptide A2 (L1-7) and an aflibercept domain that specifically binds to VEGF (A2Z-d, A2Z-ld, ZA2-s, ZA2-d, ZA2-ls and ZA2-ld), as well as exemplary bispecific antagonist configurations containing an Ang2-specific peptide A2 and the variable regions of an anti-VEGF antibody (As) (AsA2-d, AsA2-ld, AsA2-s and AsA2-ls).

FIG. 2 shows amino acid sequences of some of the functional domains used in the multispecific agonist of the present application.

FIG. 3 shows an exemplary bispecific construct A2Z-d, which contains an Fc scaffold with one or more Ang2-specific peptides A2 linked to each chain of the Fc scaffold at the N-terminal and an aflibercept domain linked to each chain of Fc scaffold at the C-terminal. The Fc scaffold may be any Fc scaffold described in the present application. Linkers of various lengths may be used between two Ang2-specific peptides A2, between the Ang2-specific peptide and the Fc scaffold, or between the aflibercept domain and the Fc scaffold. An exemplary sequence of the A2Z-d construct is shown in SEQ ID NO:8.

FIG. 4 shows an exemplary bispecific construct A2Z-ld, which contains an Fc scaffold with one or more Ang2-specific peptides A2 inserted into the CH3 loop region of each chain of the Fc scaffold and an aflibercept domain linked to each chain of the Fc scaffold at the C-terminal. The Fc scaffold may be any Fc scaffold described in the present application. Linkers of various lengths may be used between two Ang2-specific peptides A2, between the Ang2-specific peptide and the Fc scaffold, or between the aflibercept domain and the Fc scaffold. An exemplary sequence of the A2Z-ld construct is shown in SEQ ID NO:9.

FIG. 5 shows an exemplary bispecific construct ZA2-s, which contains a Fc scaffold with an aflibercept domain linked to each chain of the Fc scaffold at the N-terminal and a single Ang2-specific peptide A2 linked to each chain of the Fc scaffold at the C-terminal. The Fc scaffold may be any Fc scaffold described in the present application. Linkers of various lengths may be used between the Ang2-specific peptide and the Fc scaffold or between the aflibercept domain and the Fc scaffold. An exemplary sequence of the ZA2-s construct is shown in SEQ ID NO:10.

FIG. 6 shows an exemplary bispecific construct ZA2-d, which contains a Fc scaffold with an aflibercept domain linked to each chain of the Fc scaffold at the N-terminal and two Ang2-specific peptides A2 linked to each chain of the Fc scaffold at the C-terminal. The Fc scaffold may be any Fc scaffold described in the present application. Linkers of various lengths may be used between two Ang2-specific peptide A2, between the Ang2-specific peptide and the Fc scaffold, or between the aflibercept domain and the Fc scaffold. An exemplary sequence of the ZA2-d construct is shown in SEQ ID NO:11.

FIG. 7 shows an exemplary bispecific construct ZA2-ls, which contains a Fc scaffold with an aflibercept domain linked to each chain of the Fc scaffold at the N-terminal and one Ang2-specific peptide A2 inserted into the CH3 loop of each chain of the Fc scaffold. The Fc scaffold may be any Fc scaffold described in the present application. Linkers of various lengths may be used between two Ang2-specific peptide A2, between the Ang2-specific peptide and the Fc scaffold, or between the aflibercept domain and the Fc scaffold. An exemplary sequence of the ZA2-ls construct is shown in SEQ ID NO: 12.

FIG. 8 shows an exemplary bispecific construct ZA2-ld, which contains a Fc scaffold with an aflibercept domain linked to each chain of the Fc scaffold at the N-terminal and two Ang2-specific peptides A2 inserted into the CH3 loop of each chain of the Fc scaffold. The Fc scaffold may be any Fc scaffold described in the present application. Linkers of various lengths may be used between two Ang2-specific peptide A2, between the Ang2-specific peptide and the Fc scaffold, or between the aflibercept domain and the Fc scaffold. An exemplary sequence of the ZA2-ld construct is shown in SEQ ID NO: 13.

FIG. 9 shows an exemplary bispecific construct AsA2-d, which contains a Fc scaffold with the F(ab)₂ fragment of an anti-VEGF antibody (As) linked to the the N-terminal of the Fc scaffold and one or more Ang2-specific peptides A2 (L1-7) linked to each chain of the Fc scaffold at the C-terminal. The Fc scaffold may be any Fc scaffold described in the present application. Linkers of various lengths may be used between two Ang2-specific peptide A2, between the Ang2-specific peptide and the Fc scaffold, or between the As F(ab)₂ fragment and the Fc scaffold. Exemplary sequences of the AsA2-d construct are shown in SEQ ID NO:14 (hesvy chain) and SEQ ID NO:15 (light chain). The heavy chain variable region of antibody As is shown in SEQ ID NO:88 and the light chain variable region of antibody As is shown in SEQ ID NO:89.

FIG. 10 shows an exemplary bispecific construct AsA2-ld, which contains a Fc scaffold with the F(ab)₂ fragment of an anti-VEGFantibody (As) linked to the N-terminal of the Fc scaffold and one or more Ang2-specific peptides A2 (L1-7) inserted into the CH3 loop of each chain of the Fc scaffold. The Fc scaffold may be any Fc scaffold described in the present application. Linkers of various lengths may be used between two Ang2-specific peptide A2, between the Ang2-specific peptide and the Fc scaffold, or between the As F(ab)₂ fragment and the Fc scaffold. Exemplary sequences of the AsA2-ld construct are shown in SEQ ID NO:16 (hesvy chain) and SEQ ID NO:17 (light chain).

FIG. 11 shows additional Ang2-specific peptides A2 that can be used to. replace the L1-7 peptide in the above figures.

FIG. 12 shows molecule characterization (SEC) of anti-VEGF/Ang2 bispecific constructs A2Z-d, A2Z-ld, ZA2-s, ZA2-d, ZA2-ls and ZA2-ld. HPLC analysis was performed to evaluate the levels of aggregation and homogeneity for each protein. Briefly, 5-10 ugs of purified proteins were injected to a TOSOH UP-SW3000 size exclusion column, and the UV peaks were integrated to calculated the percent distribution of high molecular weight, main peak, and low molecular weight species and summarized in FIG. 14.

FIG. 13 shows molecule characterization (SEC) of anti-VEGF/Ang2 bispecific molecules AsA2-d, AsA2-ld, AsA2-s and AsA2-ls. HPLC analysis was performed to evaluate the levels of aggregation and homogeneity for each protein. Briefly, 5-10 ugs of purified proteins were injected to a TOSOH UP-SW3000 size exclusion column, and the UV peaks were integrated to calculated the percent distribution of high molecular weight, main peak, and low molecular weight species and summarized in FIG. 14.

FIG. 14 is a summary of aggregation profiles of the bispecific antibodies shown in FIGS. 12 and 13.

FIG. 15 is a diagram showing that exemplary bispecific molecules inhibit binding of Ang2 to its receptor Tie2. Compared to ZPT molecules (ZPT-3L/3S) which contain the long or short version of the trebananib peptide (AMG386) that inhibit both Ang2 and Ang1, A2 peptide is more potent and specifically inhibit Ang2 activity.

FIG. 16 shows the amino acid sequences of ZPT-3L/3S constructs.

FIG. 17 is another diagram showing that exemplary bispecific molecules inhibit binding of Ang2 to its receptor Tie2.

FIG. 18 is another diagram showing that exemplary bispecific molecules inhibit binding of Ang2 to its receptor Tie2.

FIG. 19 is a diagram showing that the exemplary bispecific molecules of the present application inhibit VEGF signal in cell-based assay.

FIG. 20 is another diagram showing that the exemplary bispecific molecules of the present application inhibit VEGF signal in cell-based assay.

FIG. 21 is another diagram showing that the exemplary bispecific molecules of the present application inhibit VEGF signal in cell-based assay.

FIG. 22 is a Western blot showing that A2Z-Ld and ZA2-d can also be packaged into AAV viruses and express corresponding bispecific molecules. cDNA encoding bispecific molecule A2Z-ld or ZA2-d was cloned into recombinat AAV viruses. 293 cells were transduced with recombinant AAV viruses carrying A2Z-Ld or ZA2-d cDNA for 72 hours. Culture medium were collected and 20 ul of supernatant were loaded on SDA PAGE and Western Blot were performed.

FIG. 23 shows an exemplary trispecific construct (GS01-T) that contains an PD-1 binding domain (derived from anti-PD-1 mab 2P17), an VEGF binding domain (scFv derived from anti-VEGF antibody As) and an Ang1/Ang2 bining domain (Trebananib peptide).

FIG. 24 shows another exemplary trispecific molecule (GS01-A) that contains an PD-1 binding domain (derived from anti-PD-1 mab 2P17), an VEGF binding domain (scFv derived from anti-VEGF antibody As) and an Ang2 bining domain (Ang2 binding peptide A2).

FIG. 25 is a diagram showing that the exemplary trispecific constructs of FIG. 24 and FIG. 25 inhibit VEGF signal in cell-based assay.

FIG. 26 shows SDS-PAGE characterization of trispecific constructs GS01-A and GS01-T. The trispecific contructs were expressed in HEK293 cells and purified with Protein A.

FIG. 27 shows HPLC SEC characterization of trispecific constructs GS01-A and GS01-T. The trispecific contructs were expressed in HEK293 cells and purified with Protein A.

FIG. 28A-28C shows the blocking activity of GS01-A for Ang (FIG. 28A) PD-1 (FIG. 28B) and VEGF (FIG. 28C).

FIG. 29 shows the blocking activity of GS01-T for VEGF (panel A), Ang2 (panel B) and PD-1 (panel C).

FIG. 30 shows the tumor inhibiting effect of GS01-T in hPD-1 knockin C57BL/6 mice bearing MC38.

FIG. 31 shows the tumor inhibiting effect of GS01-T in hPD-1/hTIGIT Balb/c mice bearing STK11 KO CT26.

FIG. 32 shows the pharmacokinetics of GS01-T in cynomolgus monkeys.

FIG. 33 shows exemplary bispecific VEGF and Ang2/Ang1 inhibitors Bi-TZ, Bi-ZT0, Bi-T0Z, Bi-AT0 and Bi-AmT0 for cancer therapy

FIG. 34 shows an exemplary bispecific construct Bi-TZ, which contains an Fc scaffold with trebananib peptide linked to each chain of the Fc scaffold at the N-terminal and an aflibercept domain linked to each chain of Fc scaffold at the C-terminal. The Fc scaffold may be any Fc scaffold described in the present application. Linkers of various lengths may be used between the trebananib peptide and the Fc scaffold, or between the aflibercept domain and the Fc scaffold. An exemplary sequence of the Bi-TZ construct is shown in SEQ ID NO:80.

FIG. 35 shows an exemplary bispecific construct Bi-TZ0, which contains an Fc scaffold with an aflibercept domain linked to each chain of Fc scaffold at the N-terminal and a trebananib peptide inserted into the CH3 loop of each chain of the Fc scaffold. The Fc scaffold may be any Fc scaffold described in the present application. Linkers of various lengths may be used between the aflibercept domain and the Fc scaffold. An exemplary sequence of the Bi-TZ0 construct is shown in SEQ ID NO:81.

FIG. 36 shows an exemplary bispecific construct Bi-T0Z, which contains an Fc scaffold with an aflibercept domain linked to each chain of Fc scaffold at the C-terminal and a trebananib peptide inserted into the CH3 loop of each chain of the Fc scaffold. The Fc scaffold may be any Fc scaffold described in the present application. Linkers of various lengths may be used between the aflibercept domain and the Fc scaffold. An exemplary sequence of the Bi-T0Z construct is shown in SEQ ID NO:82.

FIG. 37 shows an exemplary bispecific construct Bi-AT0, which contains an Fc scaffold with an F(ab)₂ fragment of bevacizumab linked to each chain of Fc scaffold at the N-terminal and a trebananib peptide inserted into the CH3 loop of each chain of the Fc scaffold. The Fc scaffold may be any Fc scaffold described in the present application. Linkers of various lengths may be used between the F(ab)₂ fragment of bevacizumab and the Fc scaffold. Exemplary sequences of the Bi-AT0 construct is shown in SEQ ID NO:83 (heavy chain) and SEQ ID NO:84 (light chain).

FIG. 38 shows an exemplary bispecific construct Bi-AmT0, which contains an Fc scaffold with an F(ab)₂ fragment of an improved bevacizumab linked to each chain of Fc scaffold at the N-terminal and a trebananib peptide inserted into the CH3 loop of each chain of the Fc scaffold. The Fc scaffold may be any Fc scaffold described in the present application. Linkers of various lengths may be used between the F(ab)₂ fragment of the improved bevacizumab and the Fc scaffold. Exemplary sequences of the Bi-AmT0 construct is shown in SEQ ID NO:85 (heavy chain) and SEQ ID NO:86 (light chain).

FIG. 39 shows titers of HEK 293 transient expression of bispecific molecules Bi-ZT0 and Bi-T0Z (panel A) and SEC characterization of bispecific molecules Bi-ZT0 and Bi-T0Z (panel B).

FIG. 40 shows the blocking activity of Bi-ZT0 and Bi-T0Z for VEGF (panel A) and Ang2 (panel B).

### DETAILED DESCRIPTION

Herein incorporated by reference is the sequence listing filed with the USPTO named as 2022-005 PCT.txt which was created on Jun. 3, 2021, and the size of the TXT file is 178,535 in bytes.

### I. Definitions

Unless defined otherwise, all technical and scientific terms used herein have the same meanings as commonly understood by one of skill in the art to which the disclosed method and compositions belong. It must be noted that as used herein and in the appended claims, the singular forms “a,” “an,” and “the” include plural reference unless the context clearly dictates otherwise. Thus, for example, reference to “a peptide” includes “one or more” peptides or a “plurality” of such peptides. With respect to the teachings in the present application, any issued patent or patent application publication described in this application is expressly incorporated by reference herein.

The term “agonist” refers to a substance which promotes (i.e., induces, causes, enhances, or increases) the biological activity or effect of another molecule. The term agonist encompasses substances which bind receptor, such as an antibody, and substances which promote receptor function without binding thereto (e.g., by activating an associated protein).

The term “antagonist” or “inhibitor” refers to a substance that prevents, blocks, inhibits, neutralizes, or reduces a biological activity or effect of another molecule, such as a receptor or ligand.

As used herein, the term “antibody” refers to a polypeptide or a polypeptide complex that specifically recognizes and binds to an antigen through one or more immunoglobulin variable regions. An antibody can be a whole antibody, an antigen binding fragment or a single chain thereof. The term “antibody” encompasses various broad classes of polypeptides that can be distinguished biochemically. Those skilled in the art will appreciate that heavy chains are classified as alpha, delta, epsilon, gamma, and mu, or α, δ, ε, γ and μ) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG1, IgG2, IgG3, IgG4, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are within the scope of the present disclosure, the following discussion will generally be directed to the IgG class of immunoglobulin molecules.

Antibody antagonists of the present application include immunoglobulin heavy chains paired with immunoglobulin light chains. Antibody variable domain sequences in the heavy and light chains may be from any animal origin, including birds and mammals. Preferably, the antibodies are human, murine, donkey, rabbit, goat, guinea pig, camel, llama, horse, or chicken antibodies. In some embodiments, the variable region may be condricthoid in origin (e.g., from sharks).

The terms “antibody fragment” or “antigen-binding fragment” are used with reference to a portion of an antibody, such as F(ab′)₂, F(ab)₂, Fab′, Fab, Fv, single-chain Fvs (scFv), single-chain antibodies, disulfide-linked Fvs (sdFv), fragments comprising either a VL or VH domain, fragments produced by a Fab expression library and anti-idiotypic (anti-ld) antibodies. Regardless of structure, an antibody fragment binds with the same antigen that is recognized by the intact antibody. The term “antibody fragment” includes DARTs and diabodies. The term “antibody fragment” also includes any synthetic or genetically engineered proteins comprising immunoglobulin variable regions that act like an antibody by binding to a specific antigen to form a complex. A “single-chain fragment variable” or “scFv” refers to a fusion protein of the variable regions of the heavy (VH) and light chains (VL) of immunoglobulins. In some aspects, the regions are connected with a short linker peptide of ten to about 25 amino acids. The linker can be rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker. With regard to IgGs, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration where the light chains bracket the heavy chains starting at the mouth of the “Y” and continuing through the variable region.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention, the numbering of the constant region domains in conventional antibodies increases as they become more distal from the antigen-binding site or amino-terminus of the antibody. In conventional antibodies, the N-terminal portion is a variable region and at the carboxy-terminal portion is a constant region; the CH3 and CL domains actually comprise the carboxy-terminus of the heavy and light chain, respectively. Exemplary CH1-CH2-CH3 sequences described in the current application include the following: wild type IgG1 (SEQ ID NO:92), IgG1 with a K447A mutation (SEQ ID NO:93), IgG1 with the N297A mutation (SEQ ID NO:94), wild type IgG2 (SEQ ID NO:95), IgG2 with carboxy terminal lysine deleted (SEQ ID NO:96), wild type IgG4 (SEQ ID NO:97), IgG4 with the hinge S231P mutation (SEQ ID NO:98), IgG4 with S231P and K447A (SEQ ID NO:99), and IgG4 S231P and the carboxy-terminal lysine deleted (SEQ ID NO:100). An exemplary CL sequence is set forth in SEQ ID NO:101.

The term “Fc fragment” “Fc scaffold,” and “Fc” are used with reference to a portion of an antibody contains no antigen-binding activity but was originally observed to crystallize readily, and for this reason was named the Fc fragment, for Fragment crystallizable. This fragment corresponds to the paired CH2 and CH3 domains and is the part of the antibody molecule that interacts with effector molecules and cells. The Fc fragments described herein may be derived from human IgG1, IgG2 and IgG4 antibodies with the modifications of a-glycosylation, hinge mutation and deletion of carboxy-terminal lysine. Exemplary Fc sequences described in the current application include the following: wild type IgG1 Fc (SEQ ID NO:102), a-glycosylated IgG1 Fc (SEQ ID NO:103), wild type IgG4 Fc (SEQ ID NO:104), IgG4 Fc with hinge mutation (SEQ ID NO: 105), wild type IgG2 Fc (SEQ ID NO: 106), IgG1 Fc with deletion of carboxy-terminal lysine (SEQ ID NO: 107), a-glycosylated IgG1 Fc with deletion of carboxy-terminal lysine (SEQ ID NO:108), IgG4 Fc with hinge mutation and deletion of carboxy-terminal lysine (SEQ ID NO:109), and IgG2 Fc with deletion of carboxy-terminal lysine (SEQ ID NO:110).

As indicated above, the variable region allows the antibody to selectively recognize and specifically bind epitopes on antigens. That is, the VL domain and VH domain, or subset of the complementarity determining regions (CDRs), of an antibody combine to form the variable region that defines a three dimensional antigen-binding site. This quaternary antibody structure forms the antigen-binding site present at the end of each arm of the Y. More specifically, the antigen-binding site is defined by three CDRs on each of the VH and VL chains (i.e., HCDR1, HCDR2, HCDR3, LCDR1, LCDR 2 and LCDR3). In some instances, e.g., certain immunoglobulin molecules are derived from camelid species or engineered based on camelid immunoglobulins. Alternatively, an immunoglobulin molecule may consist of heavy chains only with no light chains or light chains only with no heavy chains.

In naturally occurring antibodies, the six CDRs present in each antigen-binding domain are short, non-contiguous sequences of amino acids that are specifically positioned to form the antigen-binding domain as the antibody assumes its three dimensional configuration in an aqueous environment. The remainder of the amino acids in the antigen-binding domains, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined.

As used herein, the terms “VH1” and “VH2” refer to immunoglobulin heavy chain variable domains corresponding to two different binding specificities. Likewise, the terms “VL1” and “VL2” refer to light chain variable domains corresponding to two different binding specificities. When used together, it is to be understood that VH1 and VL1 regions define a common binding specificity and that VH2 and VL2 domains define a second binding specificity.

The term “framework region (FR)” as used herein refers to variable domain residues other than the CDR residues. Each variable domain typically has four FRs flanking the corresponding CDRs. For example, a VH domain typically has four HFRs, HFR1, HFR2, HFR3 and HFR4, flanking the three HCDRs in the configuration of HFR1-HCDR1-HFR2-HCDR2-HFR3-HCDR3-HFR4. Similarly, an LH domain typically has four LFR, LFR1, LFR2, LFR3 and LFR4, flanking the three LCDRs in the configuration of: LFR1-LCDR1-LFR2-LCDR2-LFR3-LCDR3-LFR4.

Light chains are classified as either kappa or lambda (K, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

As used herein, the term “light chain constant region (CL)” includes amino acid sequences derived from antibody light chain CL (SEQ ID NO:101). Preferably, the light chain constant region comprises at least one of a constant kappa domain or constant lambda domain.

As used herein, the term “heavy chain constant region (CH)” includes amino acid sequences derived from an immunoglobulin heavy chain. A polypeptide comprising at least one of a heavy chain constant region comprising e.g., an IgG1 CH1 (SEQ ID NO:111) or IgG4 CH1 (SEQ ID NO:112), a hinge (e.g., upper, middle, and/or lower hinge region) domain, a CH2 domain, a CH3 domain, or a variant or fragment thereof. For example, an antigen-binding polypeptide for use in the disclosure may comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In some embodiments, a polypeptide of the disclosure comprises a polypeptide chain comprising a CH3 domain. Further, an antibody for use in the disclosure may lack at least a portion of a CH2 domain (e.g., all or part of a CH2 domain). It should be understood that the heavy chain constant region may be modified such that they vary in amino acid sequence from the naturally occurring immunoglobulin molecule.

For example, Applicant has found that the CH3 domain can tolerate or accommodate significant insertions (e.g., greater than 100 aa) in the Fc loop of the CH3 domain (data not shown). Therefore, in the present application, other inhibitor domains may be similarly inserted in the Fc loop in a manner analogous to the insertion of trebananib as further described below.

The heavy chain constant region of an antibody disclosed herein may be derived from different immunoglobulin molecules. For example, a heavy chain constant region of a polypeptide may comprise a CH1 domain derived from an IgG1 molecule and a hinge region derived from an IgG3 molecule. In another example, a heavy chain constant region can comprise a hinge region derived, in part, from an IgG1 molecule and, in part, from an IgG3 molecule. In another example, a heavy chain portion can comprise a chimeric hinge derived, in part, from an IgG1 molecule and, in part, from an IgG4 molecule.

A “light chain-heavy chain pair” refers to the collection of a light chain and heavy chain that can form a dimer through a disulfide bond between the CL domain of the light chain and the CH1 domain of the heavy chain.

The subunit structures and three dimensional configurations of the constant regions of the various immunoglobulin classes are well known. As used herein, the term “VH domain” includes the amino terminal variable domain of an immunoglobulin heavy chain and the term “CH1 domain” includes the first (most amino terminal) constant region domain of an immunoglobulin heavy chain. The CH1 domain is adjacent to the VH domain and is amino terminal to the hinge region of an immunoglobulin heavy chain molecule.

As used herein the term “CH2 domain” includes the portion of a heavy chain molecule that extends, e.g., from about residue 244 to residue 360 of an antibody using conventional numbering schemes (residues 244 to 360, Kabat numbering system; and residues 231-340, EU numbering system). The CH2 domain is unique in that it is not closely paired with another domain. Rather, two N-linked branched carbohydrate chains are interposed between the two CH2 domains of an intact native IgG molecule. The CH3 domain extends from the CH2 domain to the carboxy-terminal of the IgG molecule and comprises approximately 108 residues.

As used herein, the term “hinge region” includes the portion of a heavy chain molecule that joins the CH1 domain to the CH2 domain. This hinge region comprises approximately 25 residues and is flexible, thus allowing the two N-terminal antigen-binding regions to move independently. Hinge regions can be subdivided into three distinct domains: upper, middle, and lower hinge domains.

As used herein the term “disulfide bond” includes a covalent bond formed between two sulfur atoms. The amino acid cysteine comprises a thiol group that can form a disulfide bond or bridge with a second thiol group. In most naturally occurring IgG molecules, the CH1 and CL regions are structurally linked by a disulfide bond and the two heavy chains are structurally linked by two disulfide bonds at positions corresponding to 239 and 242 using the Kabat numbering system (position 226 or 229, EU numbering system).

As used herein, a “variant” of antibody, antibody fragment or antibody domain refers to antibody, antibody fragment or antibody domain that (1) shares a sequence identity of at least 80%, 85%, 90%, 95%, 96%, 97%, 98% or 99% with the original antibody, antibody fragment or antibody domain, and (2) binds specifically to the same target that the original antibody, antibody fragment or antibody domain binds specifically. It should be understood that where a measure of sequence identity is presented in the form of the phrase “at least x % identical” or “at least x % identity”, such an embodiment includes any and all whole number percentages equal to or above the lower limit. Further it should be understood that where an amino acid sequence is presented in the present application, it should be construed as additionally disclosing or embracing amino acid sequences having a sequence identity of at least 80%, at least 85%, at least 90%, at least 95%, at least 96%, at least 97%, at least 98% or at least 99% identity to that amino acid sequence.

It should be understood that where a sequence homology range is presented herein, as in e.g., the phrase “about 80% to about 100%”, such an embodiment includes any and all sub-ranges defined by any whole numbers within, wherein the lower number can be any whole number between 80 and 100.

As used herein, the phrase “humanized antibody” refers to an antibody derived from a non-human antibody, typically a mouse monoclonal antibody. Alternatively, a humanized antibody may be derived from a chimeric antibody that retains or substantially retains the antigen binding properties of the parental, non-human, antibody but which exhibits diminished immunogenicity as compared to the parental antibody when administered to humans.

As used herein, the phrase “chimeric antibody,” refers to an antibody where the immunoreactive region or site is obtained or derived from a first species and the constant region (which may be intact, partial or modified in accordance with the instant disclosure) is obtained from a second species. In certain embodiments the target binding region or site will be from a non-human source (e.g., mouse or primate) and the constant region is human.

Included within the scope of the multispecific antibodies of the present application are various compositions and methodologies, including asymmetric IgG-like antibodies (e.g., triomab/quadroma, Trion Pharma/Fresenius Biotech); knobs-into-holes antibodies (Genentech); Cross MAbs (Roche); electrostatically matched antibodies (AMGEN); LUZ-Y (Genentech); strand exchange engineered domain (SEED) body (EMD Serono; Biolonic, Merus); Fab-exchanged antibodies (Genmab); symmetric IgG-like antibodies (e.g. dual targeting (DT)-Ig (GSK/Domantis); two-in-one antibody (Genentech); crosslinked MAbs (Karmanos Cancer Center); mAb² (F-star); Cov X-body (Cov X/Pfizer); dual variable domain (DVD)-Ig fusions (Abbott); IgG-like bispecific antibodies (Eli Lilly); Ts2Ab (Medimmune/AZ); BsAb (ZymoGenetics); HERCULES (Biogen Idec, TvAb, Roche); scFv/Fc fusions; SCORPION (Emergent BioSolutions/Trubion, ZymoGenetics/BMS); dual affinity retargeting technology (Fc-DART); MacroGenics; dual (scFv)₂-Fabs (National Research Center for Antibody Medicine); F(ab)₂ fusions (Medarex/AMGEN); dual-action or Bis-Fab (Genentech); Dock-and-Lock (DNL, ImmunoMedics); Fab-Fv (UCB-Celltech); scFv- and diabody-based antibodies (e.g., bispecific T cell engagers (BiTEs, Micromet); tandem diabodies (Tandab, Affimed); DARTs (MacroGenics); single-chain diabodies; TCR-like antibodies (AIT, Receptor Logics); human serum albumin scFv fusion (Merrimack); COMBODIES (Epigen Biotech); and IgG/non-IgG fusions (e.g., immunocytokines (EMDSerono, Philogen, ImmunGene, ImmunoMedics).

By “specifically binds” or “has specificity to”, it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D”. In some embodiments, an antibody or an antibody fragment “has specificity to” an antigen if the antibody or antibody fragment forms a complex with the antigen with a dissociation constant (K_{d}) of 10⁻⁶M or less, 10⁻⁷M or less, 10⁻⁸M or less, 10⁻⁹M or less, or 10⁻¹⁰M or less.

The term “antagonist antibody” refers to an antibody that binds to a target and prevents or reduces the biological effect of that target. In some embodiments, the term can denote an antibody that prevents the target, e.g., VEGF, to which it is bound from performing a biological function.

The phrases “dominant-negative protein” or “dominant-negative peptide” refer to a protein or peptide derived from a wild type protein that has been genetically modified by mutation and/or deletion so that the modified protein or peptide interferes with the function of the endogenous wild-type protein from which it is derived.

The phrase “VEGF binding antagonist” refers to a functional class of agents that bind to VEGF-A or its receptor, VEGFR-2, so that, as a result of the binding, activation of VEGFR-2 by VEGF-A is blocked or inhibited. As used herein, the term “VEGF binding antagonists” include antibody fragments, peptide inhibitors, dominant negative peptides and small molecule drugs, either in isolated forms or as part of a fusion protein or conjugate.

The phrase “Tie2 tyrosine kinase receptor binding antagonist” refers to a functional class of agents that bind to a Tie2 tyrosine kinase receptor or one of its ligands so that, as a result of the binding, activation of the Tie2 tyrosine kinase receptor by one or more of its ligands (i.e., Ang1, Ang2, Ang3 and Ang4) is blocked or inhibited. As used herein, the term “Tie2 tyrosine kinase receptor binding antagonist” include antibody fragments, peptide inhibitors, dominant negative peptides and small molecule drugs, either in isolated forms or as part of a fusion protein or conjugate.

The phrase “small molecule drug” refers to a molecular entity, often organic or organometallic, that is not a polymer, that has medicinal activity, and that has a molecular weight less than about 2 kDa, less than about 1 kDa, less than about 900 Da, less than about 800 Da or less than about 700 Da. The term encompasses most medicinal compounds termed “drugs” other than protein or nucleic acids, although a small peptide or nucleic acid analog can be considered a small molecule drug. Small molecules drugs can be derived synthetically, semi-synthetically (i.e., from naturally occurring precursors), or biologically.

When describing polypeptide domain arrangements with hyphens between individual domains (e.g., CH2-CH3), it should be understood that the order of the listed domains is from the amino terminal end to the carboxy terminal end.

The term “immunoconjugate” refers to an antibody which is fused by covalent linkage to an inhibitory peptide or small molecule drug. The peptide or small molecule drug can be chemically linked to the C-terminus of a constant heavy chain or to the N-terminus of a variable light and/or heavy chain.

A “linker” may be used to link the peptide or small molecule drug, such as a maytansinoid, to a multispecific antagonists in a stable, covalent manner. Linkers can be susceptible to or be substantially resistant to acid-induced cleavage, light-induced cleavage, peptidase-induced cleavage, esterase-induced cleavage, and disulfide bond cleavage, at conditions under which the compound or the antibody remains active. Suitable linkers are well known in the art and include, for example, disulfide groups, thioether groups, acid labile groups, photolabile groups, peptidase labile groups and esterase labile groups. Linkers also include charged linkers, and hydrophilic forms thereof as described herein and know in the art. The immunoconjugate may further include a flexible 3-15 amino acid linker between a multispecific antagonist and the peptide and/or small molecule drug, such as GGG (SEQ ID NO:113), GGGGS (G4S; SEQ ID NO:114) or a repetitive peptide linker thereof (SEQ ID NOS:115-118).

As used herein, the term “scaffold” refers to any polymer of amino acids that exhibits properties desired to support the function of an antagonist, including addition of antibody specificity, enhancement of antibody function or support of antibody structure and stability. A scaffold can be grafted with binding domains of a donor polypeptide to confer the binding specificity of the donor polypeptide onto the scaffold.

As used herein, the phrase “multispecific inhibitor” refers to a molecule comprising at least two targeting domains with different binding specificities. In some embodiments, the multispecific inhibitor is a polypeptide comprising a scaffold and two or more immunoglobulin antigen binding domains targeting different antigens or epitopes. In certain embodiments, the multispecific inhibitor is a bispecific antibody. In certain embodiments, the multispecific inhibitor is a trispecific antibody.

As used herein, the phrase “bispecific” refers to a molecule comprising two types of targeting domains, each with a different binding specificity. Each targeting domain is capable of binding specifically to a target molecule and inhibiting a biological function of the target molecule upon binding to the target molecule. In some embodiments, the targeting domain is a peptide. In some embodiments, the targeting domain includes an antigen binding domain or a CDR of an antibody. In some embodiments, the bispecific antagonist includes an immunoglobulin CH2-CH3 domain.

The term “bispecific antagonist” is used with reference to an antagonist that can specifically bind two different antigens (or epitopes). In some embodiments, the bispecific antagonist includes an immunoglobulin Fc receptor dimer fused to two pairs of different targeting domains. In some embodiments, the bispecific antagonist is configured as a full-length antibody that binds one antigen (or epitope) on one or both of its two binding arms (one pair of Ig HC/LC), and further includes a pair of targeting domains fused to the N-terminal or C-terminal ends of the Ig HC. In other embodiments, the bispecific antibody is configured as a full-length antibody that can bind two different antigens (or epitopes) in each of its two binding arms (two pairs of HC/LC) In these embodiments, the bispecific antibody has two identical antigen binding arms, with identical specificity and identical CDR sequences, and is bivalent for each antigen it binds to.

The term “trispecific antagonist” is used with reference to a molecule comprising three targeting domains with three different binding specificities. Each targeting domain is capable of binding specifically to a target molecule and inhibiting a biological function of the target molecule upon binding to the target molecule. In some embodiments, the trispecific antagonist is a polymeric molecule having two or more peptides. In some embodiments, the targeting domain comprises an antigen binding domain or a CDR of an antibody. In some embodiments, the trispecific antagonist is a trispecific antibody.

The terms “treat” and “treatment” refer to the amelioration of one or more symptoms associated with an eye disorder; prevention or delay of the onset of one or more symptoms of an eye disorder; and/or lessening of the severity or frequency of one or more symptoms of an eye disorder.

The phrases “to a patient in need thereof”, “to a patient in need of treatment” or “a subject in need of treatment” includes subjects, such as mammalian subjects, that would benefit from administration of the bispecific antagonist of the present disclosure for treatment of an angiogenic eye disorder.

The terms “therapeutically effective amount”, “pharmacologically effective amount”, and “physiologically effective amount” are used interchangeably to mean the amount of an bispecific antagonist that is needed to provide a threshold level of active antagonist agents in the bloodstream or in the target tissue. The precise amount will depend upon numerous factors, e.g., the particular active agent, the components and physical characteristics of the composition, intended patient population, patient considerations, and the like, and can readily be determined by one skilled in the art, based upon the information provided herein or otherwise available in the relevant literature.

The terms “improve”, “increase” or “reduce”, as used in this context, indicate values or parameters relative to a baseline measurement, such as a measurement in the same individual prior to initiation of the treatment described herein, or a measurement in a control individual (or multiple control individuals) in the absence of the treatment described herein.

A “control individual” is an individual afflicted with the same eye disorder as the individual being treated, who is about the same age as the individual being treated (to ensure that the stages of the disease in the treated individual and the control individual(s) are comparable). The individual (also referred to as “patient” or “subject”) being treated may be a fetus, infant, child, adolescent, or adult human with an eye disorder.

A “control individual” is an individual afflicted with the same cell eye disorder as the individual being treated, who is about the same age as the individual being treated (to ensure that the stages of the disease in the treated individual and the control individual(s) are comparable). The individual (also referred to as “patient” or “subject”) being treated may be a fetus, infant, child, adolescent, or adult human with an eye disorder.

### II. Multispecific Anti-Angiogenic Antagonists

Angiogenesis is a physiological process involving the growth of new blood vessels from pre-existing vessels. Angiogenesis is a normal and vital process in growth and development, as well as in wound healing and in granulation tissue. Angiogenesis is regulated by a complex and interrelated system of pathways that involve various angiogenic and angiostatic factors. Over- or under-expression of angiogenic or angiostatic factors results in various pathologic conditions, including several eye disorders characterized by aberrant angiogenesis.

The present application provides multispecific antagonist compositions and methods targeting angiogenesis pathways for treating a variety of angiogenic eye disorders characterized by aberrant angiogenesis. Two important angiogenesis pathways targeted by the multispecific antagonists of the present application include the vascular endothelial growth factor (VEGF) pathway and the Tie2 pathway. In some embodiments, the multispecific antagonists of the present application further target the TGF-β pathway.

### 1. VEGF Pathway and VEGF Pathway Antagonists

The principal VEGF pathway is mediated by the transmembrane tyrosine kinase VEGF-R2. Various isoforms of VEGF, particularly VEGF-A, bind to VEGF-R1 and VEGFF-R2, resulting in dimerization and activation through phosphorylation of various downstream tyrosine kinases.

In some embodiments, the VEGF pathway antagonist binds to VEGF-A or its receptors VEGFR-1 and VEGFR-2 so that, as a result of the binding, activation of VEGFR-1 and VEGFR-2 by VEGF-A is blocked or inhibited. Angiogenesis inhibitors may be in the form of e.g., antibodies, variable domain fragments, or dominant negative fusion protein fragments.

An exemplary dominant negative anti-VEGFR antagonist is a protein fragment corresponding to the extracellular domain (ECD) of human VEGF receptor 1 or 2. In a preferred embodiment, the dominant negative anti-VEGFR antagonist comprises an aflibercept binding domain (Zaltrap®). Aflibercept binds multiple ligands involved in angiogenesis, including VEGF-A, VEGF-B, anti-placental growth factor (PIGF)-1 and PIGF-2, including soluble ligands in circulation. As such, VEGFR ECDs, such as aflibercept act as soluble receptor decoys for VEGF-A. In certain embodiments, the VEGF binding domain comprises the amino acid sequence of SEQ ID NO:1. In other embodiments, the VEGF binding domain comprises an aglycosylated (or unglycosylated) variant sequence of SEQ ID NO:87. In certain preferred embodiments described below, an aflibercept binding domain is fused to a human IgG4 Fc fragment in the multispecific antagonists of the present application.

VEGF pathway antagonists also include anti-VEGF antibodies and small molecule inhibitors of the VEGF pathway.

Examples of anti-VEGF antibodies include, but are not limited to, antibody As, bevacizumab, ramucirumab, ranibizumab, the G6 or B20 series antibodies (e.g., G6-23, G6-31, B20-4.1) described in U.S. Publication No. 2006/0280747, 2007/0141065 and/or 2007/0020267, and the antibodies described in U.S. Pat. Nos. 7,297,334, 7,060,269, 6,884,879, 6,582,959, 6,703,020, 6,054,297; U.S. Patent Application Publication Nos. U.S. 2007/059312, U.S. 2006/009360, U.S. 2005/0186208, U.S. 2003/0206899, U.S. 2003/0190317, and U.S. 2003/0203409.

Antibody As is an antibody that binds specifically to VEGF. The HCVR and LCVR sequences of antibody As are shown in SEQ ID NOS: 88 and 89.

Bevacizumab (trade name Avastin™) comprises mutated human IgG1 framework regions (FRs) and antigen-binding complementarity-determining regions from the murine anti-hVEGF monoclonal antibody A4.6.1 that blocks binding of human VEGF-A to VEGFR-2. Approximately 93% of the amino acid sequence of bevacizumab, including most of the framework regions, is derived from human IgG1, and about 7% of the sequence is derived from the murine antibody A4.6.1. Bevacizumab has a molecular mass of about 149,000 Daltons and is glycosylated. In certain embodiments, amino acid substitutions may be included in a bevacizumab antibody as described in U.S. Pat. No. 7,575,893. Exemplary amino acid substitutions include, but are not limited to EIQ, E6Q, L11V, Q13K, L18V, R19K, A23K, or combinations thereof. The HCVR sequence of bevacizumab, a modified HCVR sequence of bevacizumab and the LCVR sequence of bevacizumab are shown in SEQ ID NOS:3-5.

Ramucirumab is a humanized IgG1 monoclonal antibody that binds to the extracellular domain of VEGFR-2, thereby blocking its interaction with VEGF-A. Additional anti-VEGFR-2 antibodies are described in U.S. Pat. Nos. 7,498,414, 6,448,077 and 6,365,157.

Exemplary small molecule antagonists of the VEGF pathway include multikinase inhibitors of VEGFR-2, including sunitinib, sorafenib, cediranib, pazonpanib and nintedanib.

### 2. Tie2 Pathway and Tie2 Pathway Antagonists

The Tie2 pathway is another angiogenesis pathway for which therapeutic antibodies and small molecule drugs have been developed. The Tie2 tyrosine kinase receptor activates angiogenesis in response to binding by its angiopoietin (Ang) ligands (i.e., Ang1, Ang2, Ang3 (mouse) and Ang4). A Tie2 pathway antagonist binds to the Tie2 tyrosine kinase receptor or one of its angiopoietin (Ang) ligands (i.e., Ang-1, Ang-2, Ang-3 and Ang-4) so that, as a result of the binding, activation of the Tie2 receptor by one or more of its ligands is blocked or inhibited.

Tie2 pathway antagonists include, but are not limited to, Tie2 receptor binding antagonists, small molecule inhibitors and antibody-based antagonists.

Examples of Tie2 receptor binding antagonists include, but are not limited to peptide antagonist such as trebananib. Unless otherwise noted, the “trebananib” or “trebananib peptide” has the amino acid sequence of AQQEECEWDPWTCEHMGSGSATGGSGSTASSGSGSATHQEECEWDPWTCEHM (SEQ ID NO:2). Trebananib selectively binds to both Ang2 and Ang1, inhibiting their interactions with Tie2.

In other embodiments, the Tie2 receptor binding antagonist is a Ang2-specific binding peptide (peptide A2). Examples of peptide A2 include, but are not limited to, peptides shown in SEQ ID NOS:6, 7 and 18-73. Other peptide inhibitors of Tie2 activation (including Ang-2 inhibitors) for use in the bispecific antagonists of the present application include A-11 (Compugen), which comprises the amino acid sequence ETFLSTNKLENQ (SEQ ID NO:119); a peptide having the amino acid sequence NSLSNASEFRAPY (SEQ ID NO:120); a peptide having the amino acid sequence NLLMAAS (SEQ ID NO:121); the CVX-060 peptide (Pfizer); the CVX-037 peptide (Pfizer); and CGEN-25017 (Compugen). Additional peptide inhibitors of Tie2 activation are described in U.S. Pat. No. 7,138,370. Exemplary peptide inhibitors of angiopoietin-1 or angiopoietin-2 are described in U.S. Pat. Nos. 7,138,370, 7,521,053, 7,658,924, and 8,030,025.

Examples of small molecule Tie2 pathway antagonists include, but are not limited to, CGI-1842 (CGI Pharmaceuticals), LP-590 (Locus Pharmaceuticals), ACTB-1003 (Act Biotech/Bayer AG), CEP-11981 (Cephalon/Teva), MGCD265 (Methylgene), Regorafenib (Bayer), Cabozantinib/XL-184/BMS-907351 (Exelixis), Foretnib (Exelixis), and MGCD-265 (MethylGene Inc.).

Antibody-based Tie2 pathway antagonists include, but are not limited to, AMG-780 (Amgen), MEDI-3617 (MedImmune/AstraZeneca), DX-2240 (Dyax/Sanofi-Aventis), REGN-910 (Sanofi/Regeneron), RG7594 (Roche), LC06 (Roche), TAvi6 (Roche), AT-006 (Roche/Affitech), as well as HCVRs, LCVRs, HCs and LCs thereof. Additional Tie2 receptor binding antibody antagonists and antibody binding sequences therefrom are described in U.S. Pat. Nos. 6,376,653, 7,521,053, 7,658,924, 8,030,025, as well as U.S. Patent Application Publication Nos. 2013/0078248, 2013/0259859, and 2015/0197578.

### 3. Bispecific Constructs

One aspect of the present application relates to a bispecific antagonist that comprises: a protein scaffold comprising a Fc fragment of an immunoglobulin, a first targeting domain that binds specifically to VEGF or VEGFR and a second targeting domain comprising an inhibitor of the angiopoietin/Tie-2 signaling pathway.

In some embodiments, the first targeting domain is structurally linked to the N-terminus of the protein scaffold and the second targeting domain is structurally linked to the C-terminus of the protein scaffold.

In some embodiments, the first targeting domain is structurally linked to the C-terminus of the protein scaffold and the second targeting domain is structurally linked to the N-terminus of the protein scaffold.

In some embodiments, the first targeting domain is located at the N-terminal or C-terminal of the protein scaffold and the second targeting domain is inserted in a CH3 domain in an Fc loop of the protein scaffold.

In some embodiments, the Fc fragment is derived from human IgG1, human IgG2 or human IgG4. In some embodiments, the Fc fragment is selected from SEQ ID NOS: 102-110. In some embodiments, the Fc fragment is selected from SEQ ID NOS: 102-110 prior to the insertion of the second targeting domain.

In some embodiments, the first targeting domain comprises an aflibercept VEGF binding domain. In some embodiments, the first targeting domain comprises SEQ ID NO:1.

In some embodiments, the first targeting domain comprises the VEGF binding domain of antibody As. In some embodiments, the first targeting domain comprises SEQ ID NOS:88 and 89.

In some embodiments, the first targeting domain comprises a bevacizumab VEGF binding domain or a variant/mutant thereof. In some embodiments, the first targeting domain comprises SEQ ID NOS:122 and 124. In some embodiments, the first targeting domain comprises SEQ ID NOS: 123 and 124.

In some embodiments, the second targeting domain comprises one or more copies of Ang2-specific peptide. In some embodiments, the Ang2-specific peptide is selected from the group consisting of SEQ ID NOS 6, 7 and 18-73. In some embodiments, the second targeting domain comprises SEQ ID NO:6 or 7.

In some embodiments, the second targeting domain comprises a trebanaib peptide.

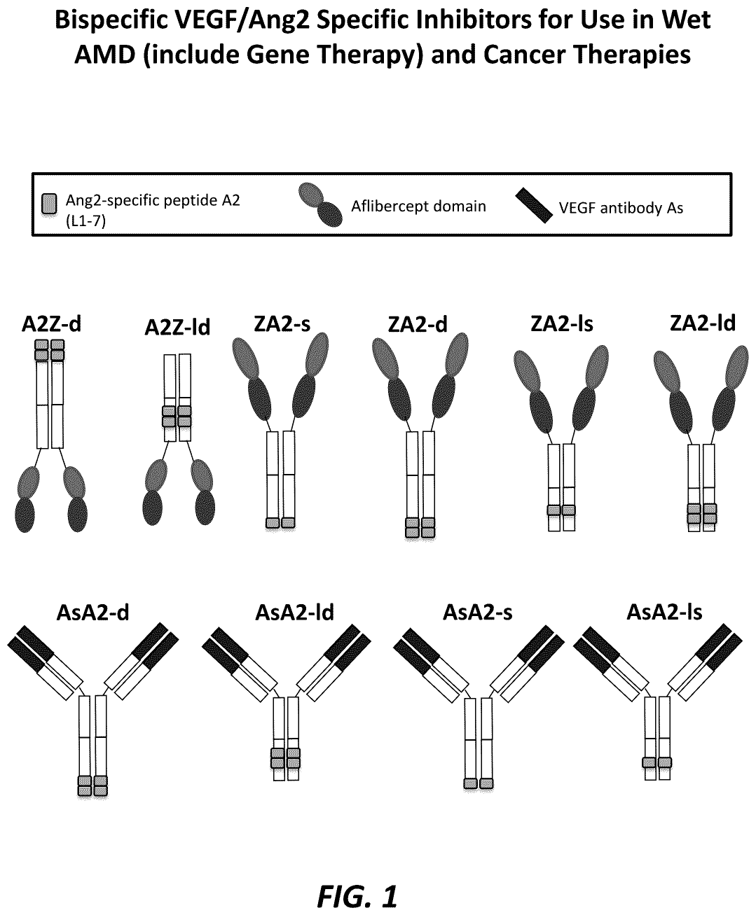

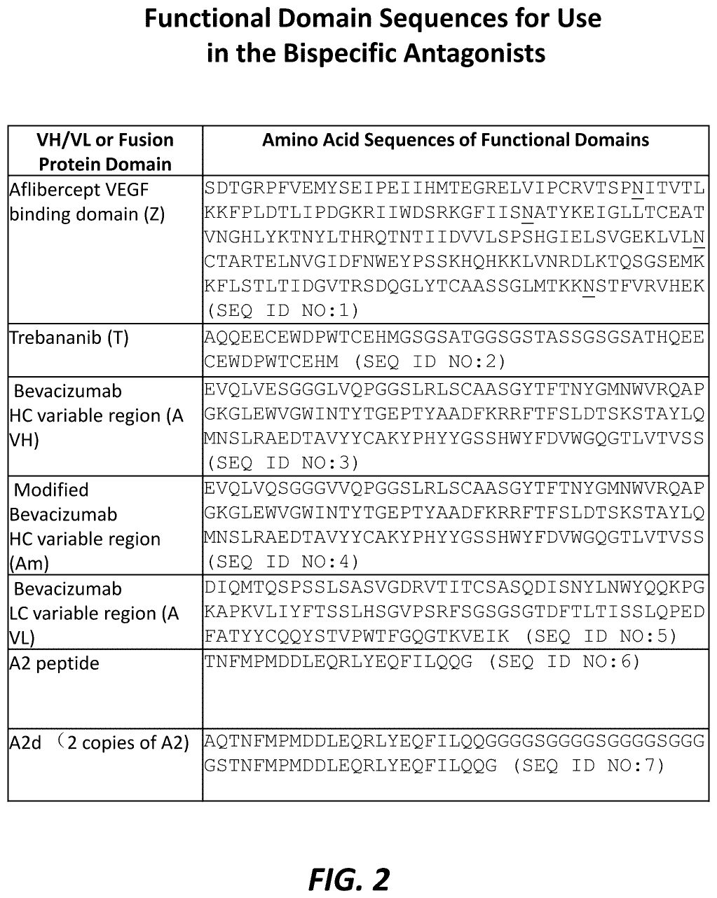

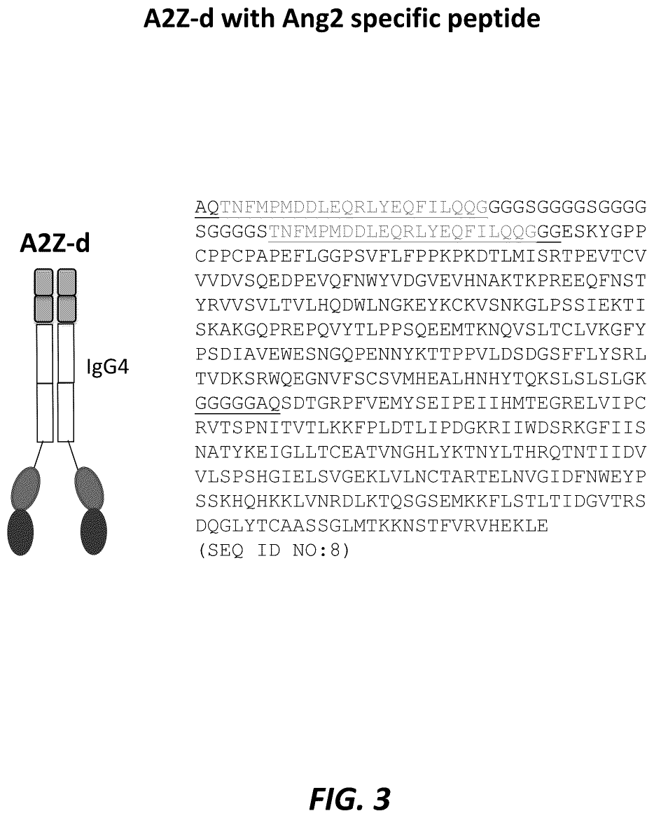

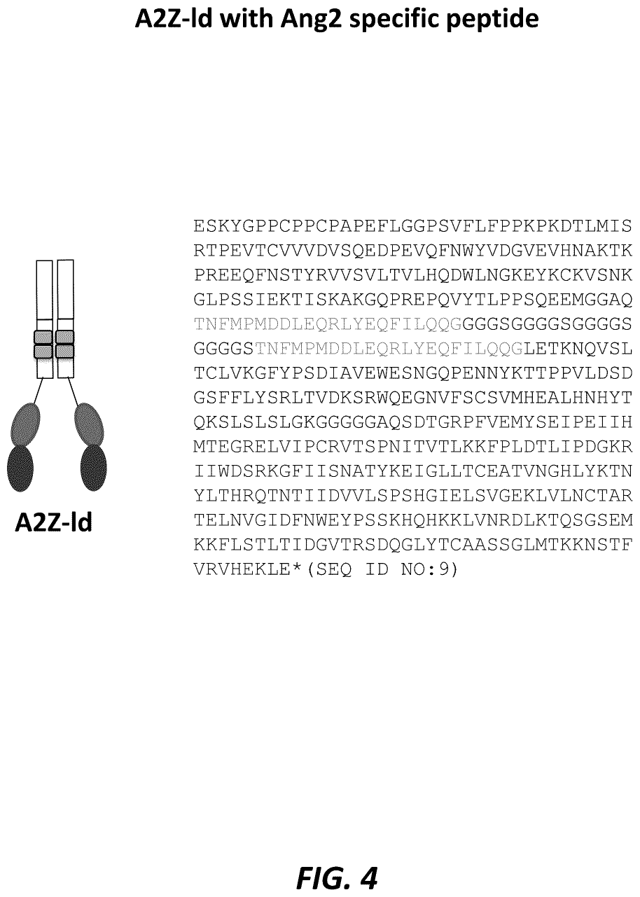

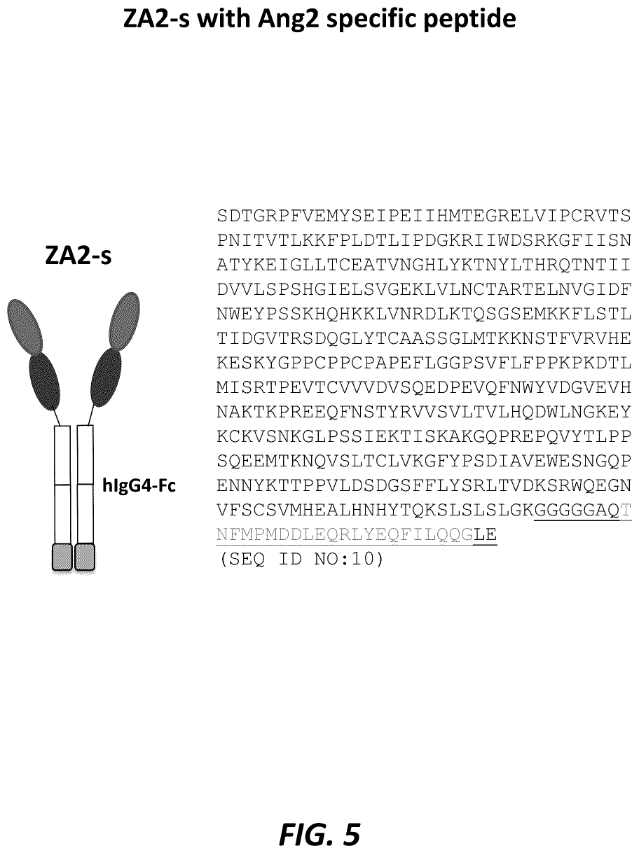

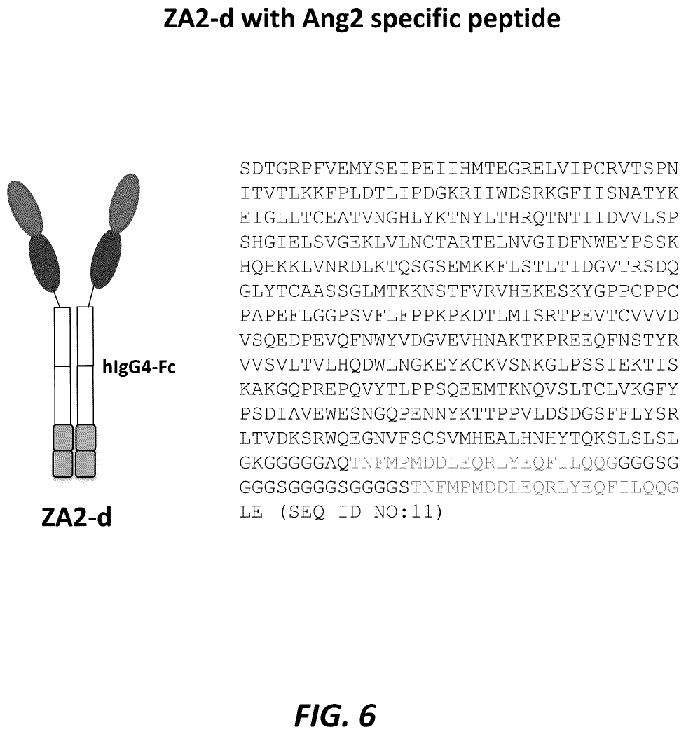

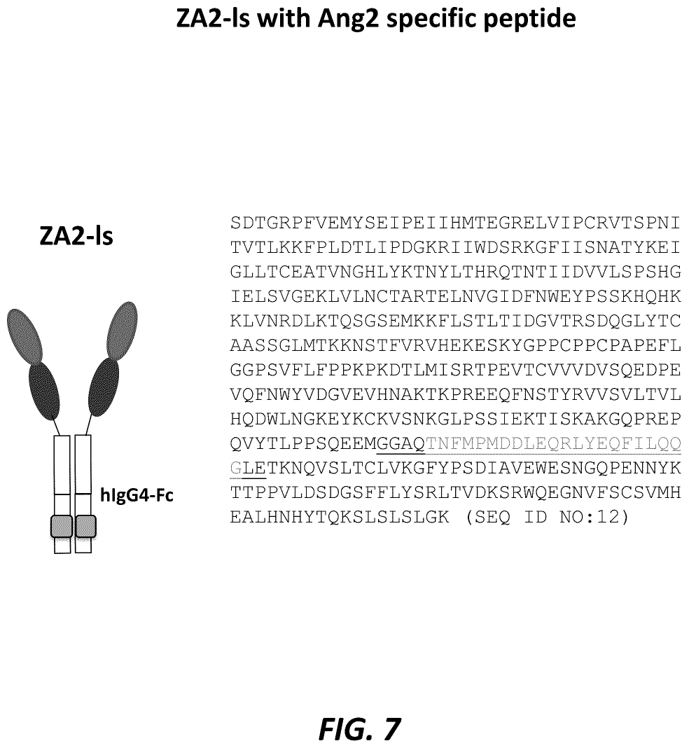

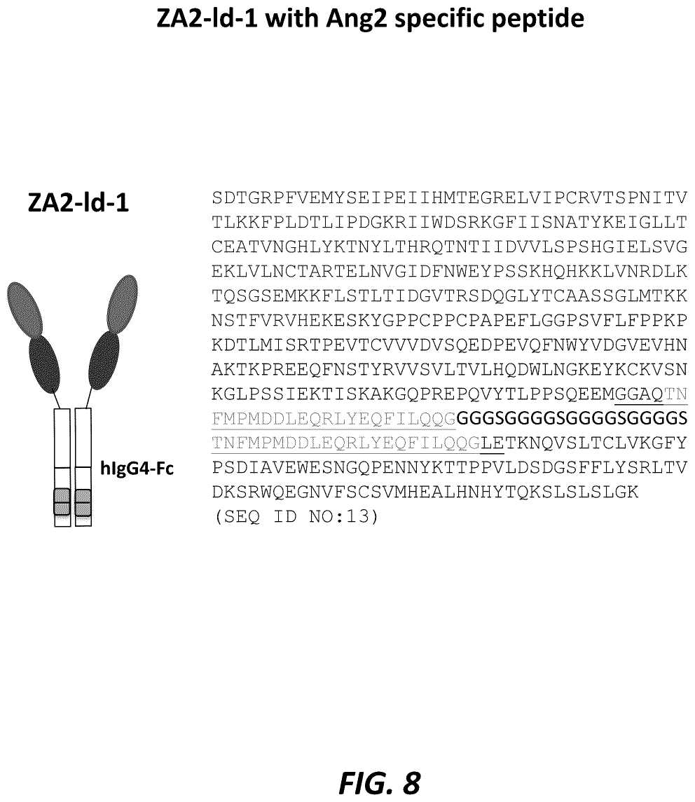

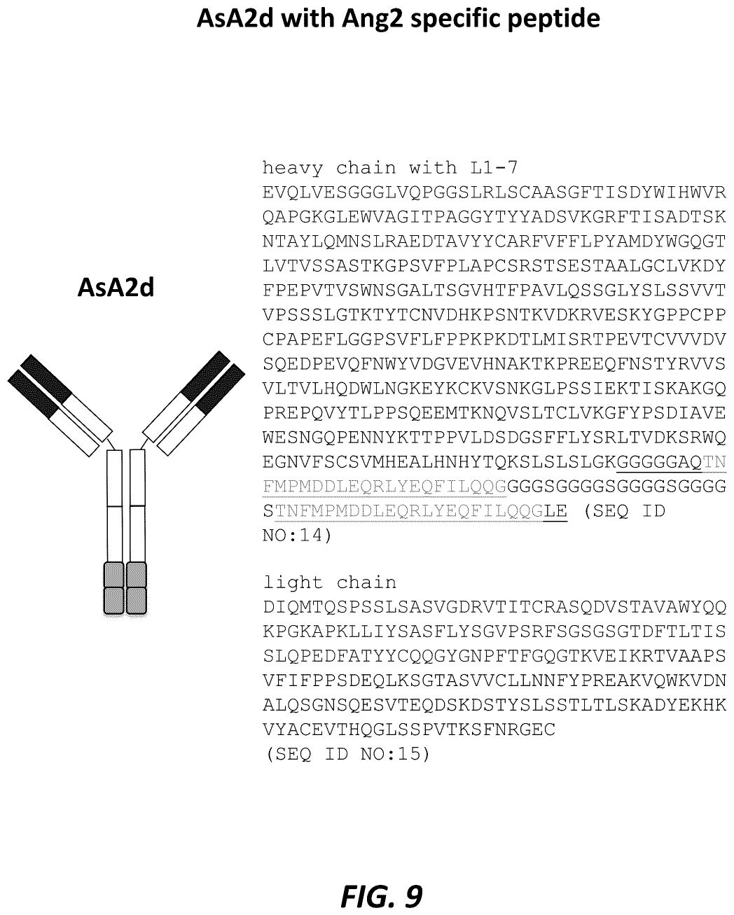

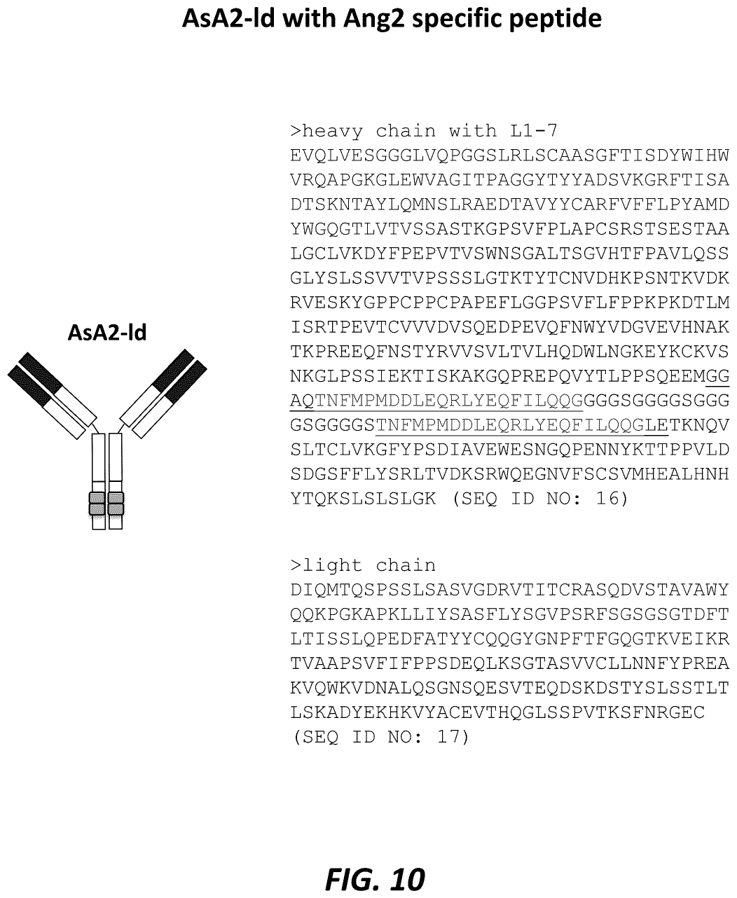

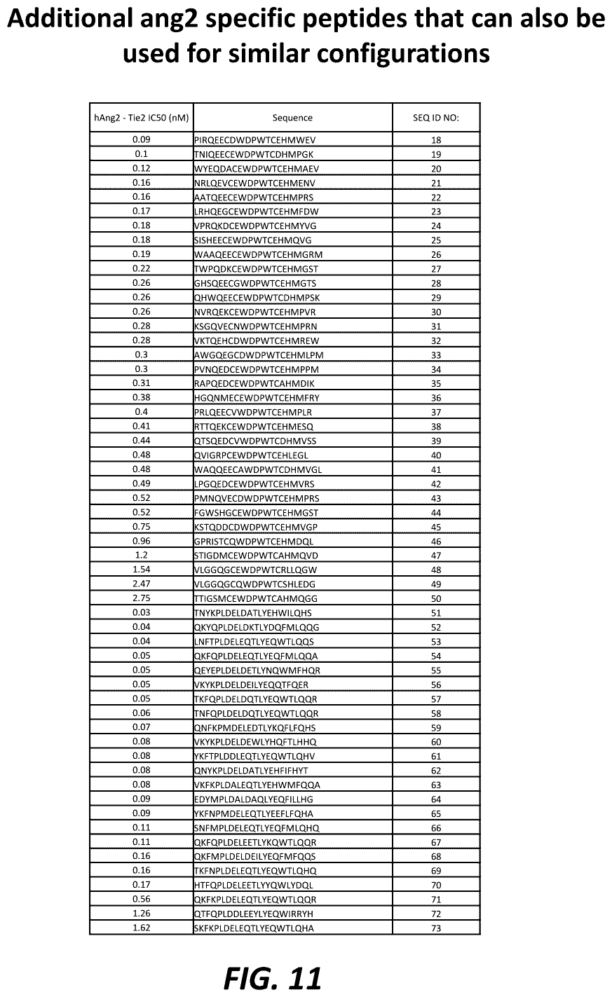

FIG. 1 shows exemplary bispecific antagonist configurations with a VEGF binding domain and one or more Ang-2 specific peptide. FIGS. 3-10 show the amino acid sequences corresponding to the eight bispecific antagonist configurations in FIG. 1.

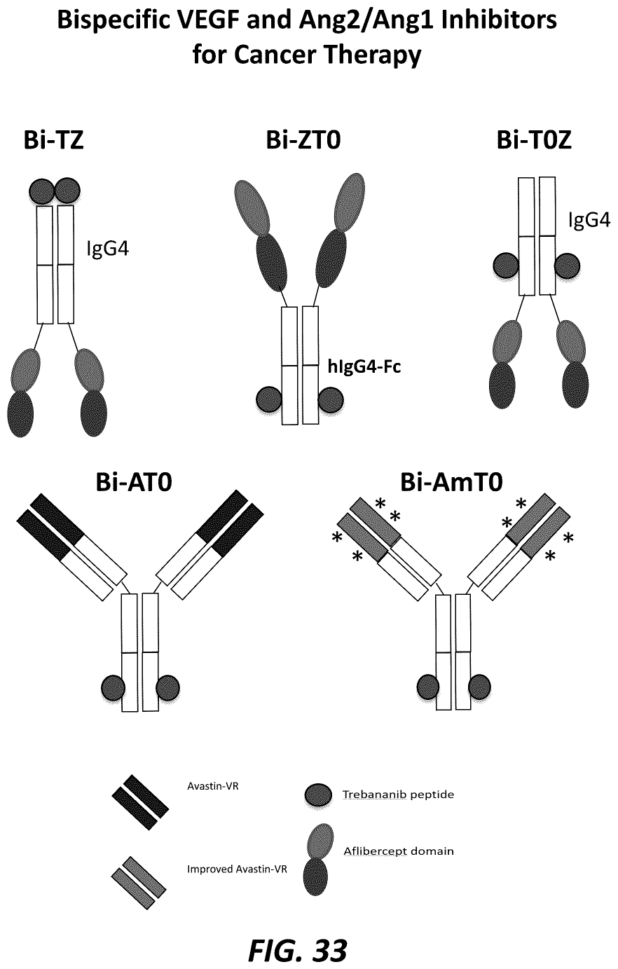

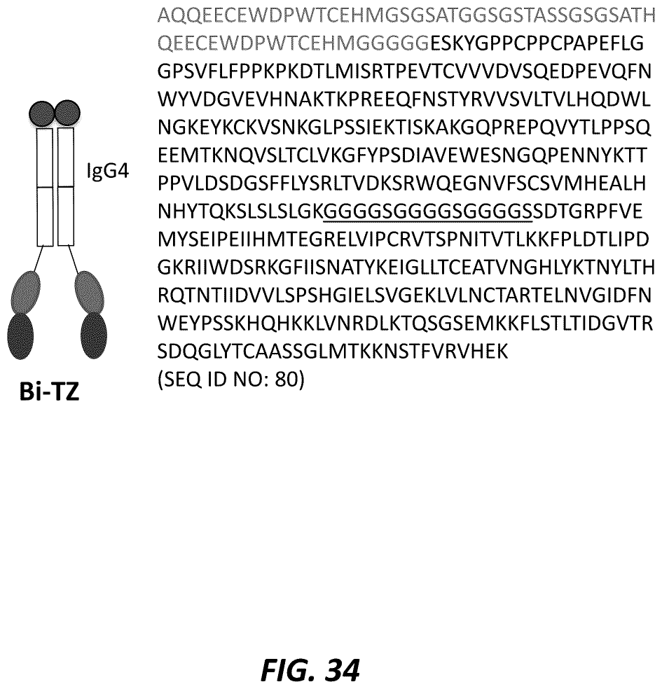

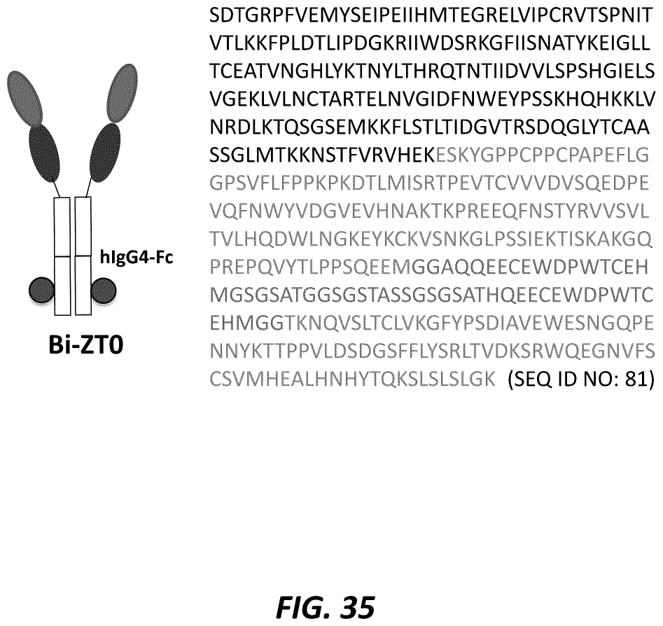

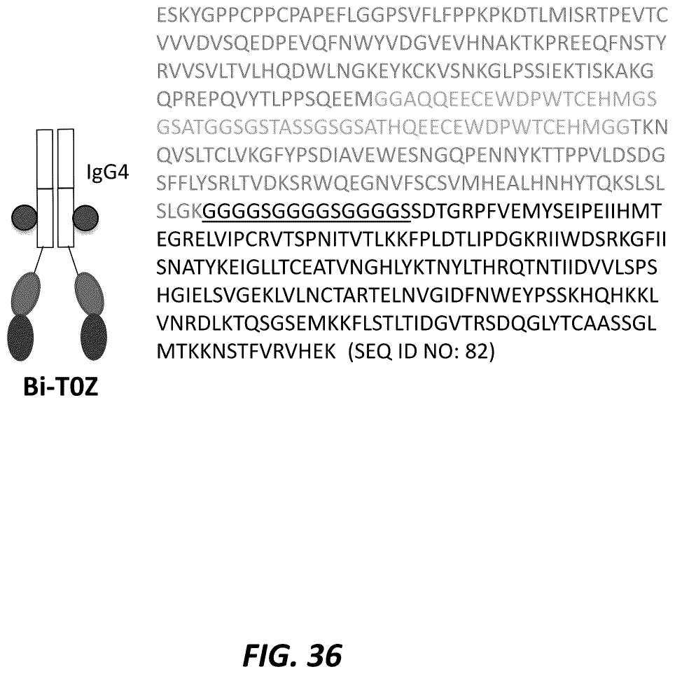

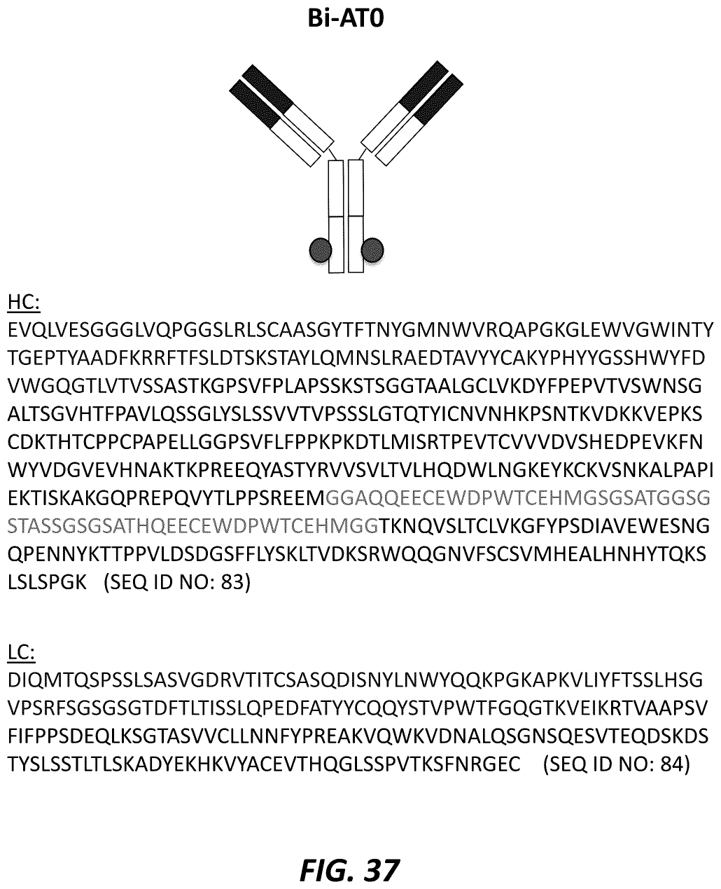

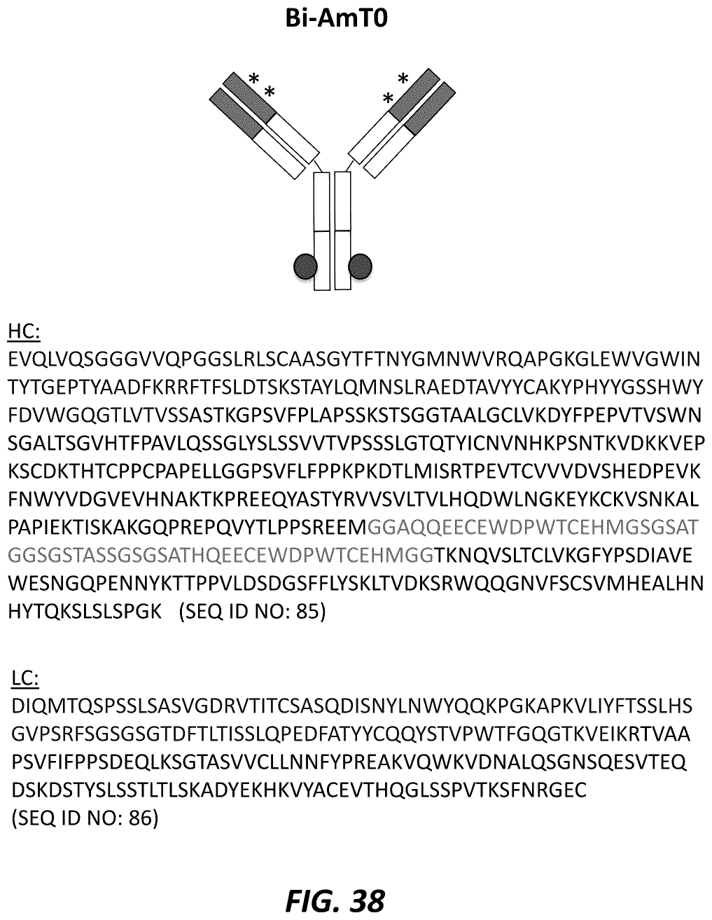

FIG. 33 shows exemplary bispecific antagonist configurations with VEGF binding domain and a trebananib peptide. FIGS. 34-38 show the amino acid sequences corresponding to the five bispecific antagonist configurations in FIG. 33

### 4. Trispecific Constructs

Another aspect of the present application relates to a trispecific antagonist that comprises: a protein scaffold comprising a Fc fragment of an immunoglobulin, a first targeting domain that binds specifically to VEGF or VEGFR, a second targeting domain comprising an inhibitor of the angiopoietin/Tie-2 signaling pathway, and a third target domain that binds specifically to PD-1.

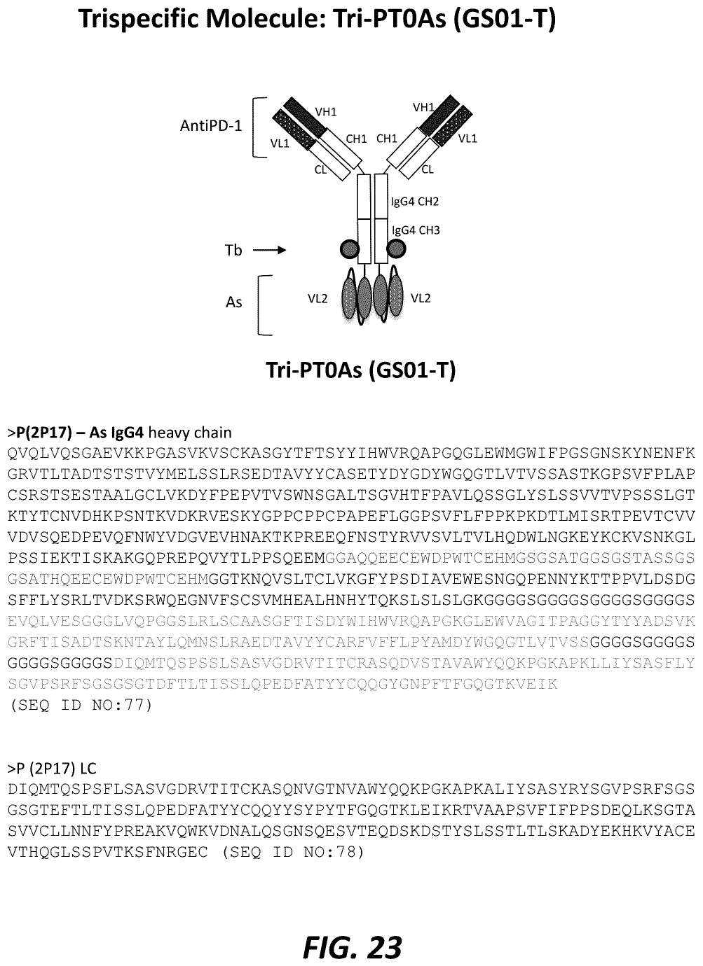

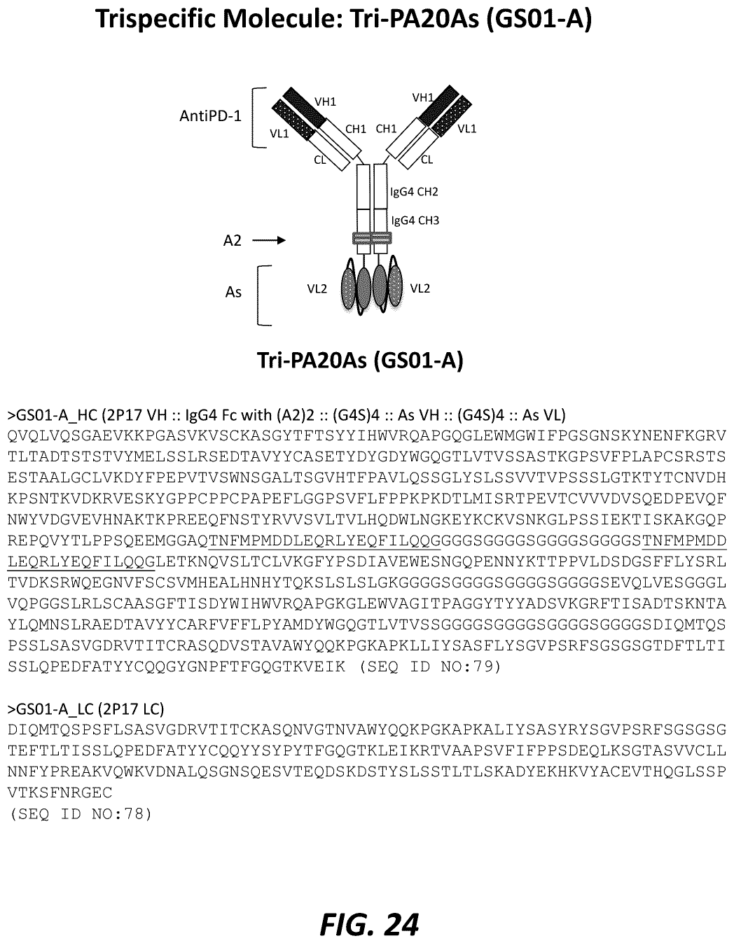

FIGS. 23 and 24 show two exemplary trispecific constructs of the present application. Trispecifc construct GS01-T (FIG. 23) contains the PD-1 binding domain of anti-PD-1 antibody 2P-17, a trebananib peptide inserted into the CH3 loop of each chain of the IgG4 Fc fragment, and an scFv converted from the anti-VEGF antibody As at the C-terminal of each chain of the Fc fragment. Trispecifc construct GS01-A (FIG. 24) contains the PD-1 binding domain of anti-PD-1 antibody 2P-17, an anti-Ang2 peptide A2 inserted into the CH3 loop of each chain of the IgG4 Fc fragment, and an scFv converted from the anti-VEGF antibody As at the C-terminal of each chain of the Fc fragment.

### 5. Modifications to the Multispecific Constructs of the Present Application

The asialoglycoprotein receptor 1 and 2 (ASGR1 and ASGR2) is transmembrane proteins that play a critical role in serum glycoprotein homeostasis by mediating the endocytosis and lysosomal degradation of glycoproteins with exposed terminal galactose or N-acetylgalactosamine residues. In some cases, glycosylation of proteins can lead to increased clearance by ASGR1 and ASGR2.

To improve the pharmacokinetics (i.e., achieve longer half-life) of the multispecific antagonists with VEGF binding domains, the inventors of the present application have created an aglycosylated variant of the VEGF binding component for limiting protein clearance by ASGR1 and ASGR2. In some embodiments, the aflibercept domain of the multispecific antagonists described herein is aglycosylated due to the replacement of asparagine (Asn, N) residues with glutamic acid (Glu, E) residues. N-linked glycosylation is the attachment of a glycan, to a nitrogen atom (the amide nitrogen) of an Asn residue of a protein. The Asn residue to be glycosylated must be located in a specific consensus sequence in the primary structure (Asn-X-Ser or Asn-X-Thr, where X refers to any amino acid except proline). By replacing the Asn residues with Glu residues (or any other amino acid residues), no glycosylation occurs on these sites. One or more Asn residues located in the specific consensus sequence described above may be replaced.

The multispecific antagonist variant with an aglycosylated VEGFR component retains the VEGF binding bioactivity and has been found to improve the alpha phase of pharmacokinetics compared to a glycosylated VEGF binding component (data not shown). An exemplary amino acid sequence of an aglycosylated aflibercept VEGF binding domain is shown in SEQ ID NO:87, i.e., in SDTGRPFVEMYSEIPEIIHMTEGRELVIPCRVTSPEITVTLKKFPLDTLIPDGKRIIWDSRKG FIISEATYKEIGLLTCEATVNGHLYKTNYLTHRQTNTIIDVVLSPSHGIELSVGEKLVLECT ARTELNVGIDFNWEYPSSKHQHKKLVNRDLKTQSGSEMKKFLSTLTIDGVTRSDQGLYT CAASSGLMTKKESTFVRVHEK. The replacement sites are underlined.

An exemplary amino acid sequence of a glycosylated aflibercept VEGF binding domain is shown in SEQ ID NO:1, i.e., SDTGRPFVEMYSEIPEIIHMTEGRELVIPCRVTSPNITVTLKKFPLDTLIPDGKRIIWDSRKG FIISNATYKEIGLLTCEATVNGHLYKTNYLTHRQTNTIIDVVLSPSHGIELSVGEKLVLNC TARTELNVGIDENWEYPSSKHQHKKLVNRDLKTQSGSEMKKFLSTLTIDGVTRSDQGLY TCAASSGLMTKKNSTFVRVHEK. The glycosylation sites are underlined.

Fc fusion proteins produced by recombinant DNA technology often face a serious problem of fully or partially degradation in cell culture (clipping) because of the host-cell derived proteases. Clipping generates low molecular weight (LMW) species of the target protein and leads to inactive polypeptides. While not wishing to be bound by any particular theory, it has been hypothesized that the location of the biological peptide affects the degree of degradation in the resulting fusion protein. The inventors of the present application found that insertion of the second targeting domain into the CH3 loop region of the Fc fragment significantly reduces clipping of the resulting multispecific antagonist.

In some embodiments, the inhibitory peptide is incorporated within Fc loop regions in each of the two CH3 regions in the immunoglobulin protein scaffold. The loop region is an irregular secondary structure in proteins that is not α-helix or ß-sheet, while it connects together β-sheets to β-sheets, β-sheets to α-helices, or α-helices to α-helices. The inhibitory peptide may be added by insertion (i.e., between amino acids in the previously existing Fc loop) or by replacement of amino acids in the previously existing Fc loop (i.e., removing amino acids in the previously existing Fc loop and adding peptide amino acids).

Any one of the binding domains corresponding to an anti-VEGF, anti-VEGFR, anti-angiopoietin-1/2, and/or anti-Tie2 receptor binding domain can be included in the bispecific antagonists of the present application. In addition, any of the antagonists described herein may include multiple binding specificities targeting VEGF, VEGFR, angiopoietin, and/or the Tie2 receptor. Moreover, any of the antibody antagonists may be engineered to target multiple epitopes in a given target.

The binding domains, including HCVRs and LCVRs described herein may be structurally linked to a protein scaffold in the form of a naturally-occurring CH1-CH2-CH3 or Fc (CH2-CH3) region or a non-naturally occurring or mutated Fc (CH2-CH3) region, e.g., an effectorless or mostly effectorless Fc (e.g., human IgG2 or IgG4). Accordingly, in certain embodiments the binding domains, including HCVRs and LCVRs, may be structurally linked to an CH1-CH2-CH3 or CH2-CH3 Ig scaffold comprising one or more modifications, typically to alter one or more functional properties of the antibody, such as serum half-life, complement fixation, Fc receptor binding, and/or antigen-dependent cellular cytotoxicity. Furthermore, an antibody described herein may be chemically modified (e.g., one or more chemical moieties can be attached to the antibody) or it may be modified to alter its glycosylation, to alter one or more functional properties of the antibody. More specifically, in certain embodiments, the antibodies in the present application may include modifications in the Fc region in order to generate an Fc variant with (a) increased or decreased antibody-dependent cell-mediated cytotoxicity (ADCC), (b) increased or decreased complement mediated cytotoxicity (CDC), (c) increased or decreased affinity for C1q and/or (d) increased or decreased affinity for a Fc receptor relative to the parent Fc. Such Fc region variants will generally comprise at least one amino acid modification in the Fc region. Combining amino acid modifications is thought to be particularly desirable. For example, the variant Fc region may include two, three, four, five, etc. substitutions therein, e.g., of the specific Fc region positions identified herein.

For uses where effector function is to be avoided altogether, e.g., when antigen binding alone is sufficient to generate the desired therapeutic benefit, and effector function only leads to (or increases the risk of) undesired side effects, IgG4 antibodies may be used, or antibodies or fragments lacking the Fc region or a substantial portion thereof can be devised, or the Fc may be mutated to eliminate glycosylation altogether (e.g., N297A). Alternatively, a hybrid construct of human IgG2 (CH1 domain and hinge region) and human IgG4 (CH2 and CH3 domains) may be generated that is devoid of effector function, lacking the ability to bind FcγRs (like IgG2) and activate complement (like IgG4). When using an IgG4 constant domain, it is usually preferable to include the substitution S228P, which mimics the hinge sequence in IgG1 and thereby stabilizes IgG4 molecules, reducing Fab-arm exchange between the therapeutic antibody and endogenous IgG4 in the patient being treated.

In certain embodiments, the binding domains, including HCVRs and LCVRs and other fragments described herein may be modified to increase its biological half-life. Various approaches may be employed, including e.g., those that decrease the binding affinity of the Fc region for FcRn. In one embodiment, the antibody is altered within the CH1 or CL region to contain a salvage receptor binding epitope taken from two loops of a CH2 domain of an Fc region of an IgG, as described in U.S. Pat. Nos. 5,869,046 and 6,121,022.

The numbering of residues in the Fc region is that of the EU index. Sequence variants disclosed herein are provided with reference to the residue number followed by the amino acid that is substituted in place of the naturally occurring amino acid, optionally preceded by the naturally occurring residue at that position. Where multiple amino acids may be present at a given position, e.g., if sequences differ between naturally occurring isotypes, or if multiple mutations may be substituted at the position, they are separated by slashes (e.g., “X/Y/Z”).

Exemplary Fc variants that increase binding to FcRn and/or improve pharmacokinetic properties include substitutions at positions 259, 308, and 434, including for example 259I, 308F, 428L, 428M, 434S, 434H, 434F, 434Y, and 434M. Other variants that increase Fc binding to FcRn include: 250E, 250Q, 428L, 428F, 250Q/428L (Hinton et al., 2004, J. Biol. Chem. 279(8): 6213-6216, Hinton et al. 2006 Journal of Immunology 176:346-356), 256A, 272A, 305A, 307A, 31 IA, 312A, 378Q, 380A, 382A, 434A (Shields et al. (2001) J. Biol. Chem., 276(9):6591-6604), 252F, 252Y, 252W, 254T, 256Q, 256E, 256D, 433R, 434F, 434Y, 252Y/254T/256E, 433K/434F/436H (Dall'Acqua et al. (2002) J. Immunol., 169:5171-5180, Dall'Acqua et al. (2006) J. Biol. Chem., 281:23514-23524, and U.S. Pat. No. 8,367,805.

Modification of certain conserved residues in an IgG Fc (1253, H310, Q311, H433, N434), such as the N434A variant (Yeung et al. (2009) J. Immunol. 182:7663), have been proposed as a way to increase FcRn affinity, thus increasing the half-life of the antibody in circulation (WO 98/023289). The combination Fc variant comprising M428L and N434S has been shown to increase FcRn binding and increase serum half-life up to five-fold (Zalevsky et al. (2010) Nat. Biotechnol. 28:157). The combination Fc variant comprising T307A, E380A and N434A modifications also extends half-life of IgG1 antibodies (Petkova et al. (2006) Int. Immunol. 18:1759). In addition, combination Fc variants comprising M252Y-M428L, M428L-N434H, M428L-N434F, M428L-N434Y, M428L-N434A, M428L-N434M, and M428L-N434S variants have also been shown to extend half-life (U.S. 2006/173170). Further, a combination Fc variant comprising M252Y, S254T and T256E was reported to increase half-life-nearly 4-fold. Dall'Acqua et al. (2006) J. Biol. Chem. 281:23514.

### IV. Pharmaceutical Compositions and Methods of Treatment

Angiogenesis inhibition constitutes a useful strategy for treating angiogenic disorders of the eye, such as wet macular degeneration, diabetic retinopathy, retinopathy of prematurity, restenosis following glaucoma treatment and neovascular glaucoma, and corneal neovascularization. Accordingly, one aspect of the present application relates to a pharmaceutical composition for treating an angiogenic disorder of the eye, characterized by aberrant or excessive angiogenesis.

In one embodiment, the pharmaceutical composition includes one or more multispecific antagonists of the present application in combination with one or more pharmaceutically acceptable carriers. Pharmaceutical compositions of the present application may include one or more different antagonists, one or more immunoconjugates, one or more other therapeutic agents, or a combination thereof as described herein. A multispecific antagonist of the present application can be used alone to inhibit angiogenesis or can be used in conjunction with another therapeutic agent as described below. The bispecific antagonist may include chimeric mouse-human binding sequences or humanized binding sequences. When the bispecific antagonist is administered together with another antagonist or therapeutic agent, the two can be administered before, during or after the bispecific antagonist.

In another aspect, a method for treating an eye disorder comprises administering to a subject in need thereof an effective amount of the pharmaceutical composition according to the present disclosure. In particular, the pharmaceutical compositions of the present application can be administered in vivo to human subjects to inhibit angiogenesis in a variety of eye diseases where downregulation of angiogenesis is desired. Accordingly, in preferred embodiments the present application provides methods of treating a subject with an eye disorder characterized by aberrant angiogenesis comprising administering to the subject a bispecific antagonist as described herein such that angiogenesis is down-regulated.

The treatment methods are particularly suitable for treating human patients with an angiogenic disorder of the eye, characterized by aberrant or excessive angiogenesis. Exemplary eye disorders for treatment with the pharmaceutical compositions described herein include any intraocular or external angiogenic disorder of the eye. For example, intraocular angiogenic disorders include disorders inside the eye, such as diabetic retinopathy, wet macular degeneration, retinopathy of prematurity, choroidal neovascularization, and neovascular glaucoma. External angiogenic disorders of the eye are exterior to the eye and include, for example, corneal neovascularization. Additional eye disorders for treatment include conditions resulting from aberrant pathologic angiogenesis resulting from medical interventions, such as restenosis following glaucoma treatment and angiogenesis resulting from corneal transplantation or cataract surgery.

In some embodiments, the bispecific antagonists of the present application are administered to cells in culture, followed by administration of the transduced cells to human subjects ex vivo to inhibit angiogenesis in an eye disease where downregulation of angiogenesis is desired.

In certain embodiments, angiogenesis inhibition can also be combined with conventional treatment regimens for the aforementioned eye disorders, including laser surgery, laser surgery in combination with a targeting drug (e.g., photodynamic therapy) and brachytherapy. In some embodiments, the bispecific antagonist may be combined with another therapeutic agent. Other combination therapies that may result in synergy with angiogenesis inhibition include co-administration with an anti-PDGFR antibody, an anti-ALK-1 antibody, an anti-FGFR1 antibody, an anti-FGFR2 antibody, an anti-TGF-β RI antibody, an anti-TGF-β RII antibody, anti-TGF-1 antibody, combinations thereof, or any small molecule antagonist directed against the foregoing targets.

Suitable dosages of the molecules used may depend on the age and weight of the subject and the concentration and/or formulation of the pharmaceutical composition. Any suitable route or mode of administration can be employed for providing the patient with a therapeutically or prophylactically effective dose of the bispecific antagonist composition. Exemplary routes or modes of administration include parenteral (e.g., intraocular, intravenous, intraarterial, intramuscular, subcutaneous), oral, topical (nasal, transdermal, intradermal or intraocular), mucosal (e.g., nasal or sublingual), or inhalation.

An intraocular injection may be administered by intravitreal injection (into the vitreous chamber behind the lens) or by intracameral injection (into the anterior chamber of the eye in front of the lens). In preferred embodiments for treatment of eye disorders, the multispecific antagonist of the present application are administered by intravitreal injection.

A pharmaceutical composition comprising a bispecific antagonist in accordance with the present disclosure may be formulated in any pharmaceutically acceptable carrier(s) or excipient(s). As used herein, the term “pharmaceutically acceptable carrier” includes any and all solvents, dispersion media, coatings, antibacterial and antifungal agents, isotonic and absorption delaying agents, and the like that are physiologically compatible. Pharmaceutical compositions may comprise suitable solid or gel phase carriers or excipients. Exemplary carriers or excipients include but are not limited to, calcium carbonate, calcium phosphate, various sugars, starches, cellulose derivatives, gelatin, and polymers such as polyethylene glycols. Exemplary pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the therapeutic agents.

The bispecific antagonist can be incorporated into a pharmaceutical composition suitable for parenteral administration. Suitable buffers include but are not limited to, sodium succinate, sodium citrate, sodium phosphate or potassium phosphate. Sodium chloride can be used to modify the toxicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 2-4%). Stabilizers can be used in both liquid and lyophilized dosage forms, principally 1-50 mM L-Methionine (optimally 5-10 mM). Other suitable bulking agents include glycine, arginine, can be included as 0-0.05% polysorbate-80 (optimally 0.005-0.01%). Additional surfactants include but are not limited to polysorbate 20 and BRIJ surfactants.

Therapeutic bispecific antagonist preparations can be lyophilized and stored as sterile powders, preferably under vacuum, and then reconstituted in bacteriostatic water (containing, for example, benzyl alcohol preservative) or in sterile water prior to injection. Pharmaceutical composition may be formulated for parenteral administration by injection e.g., by bolus injection or continuous infusion.

The therapeutic agents in the pharmaceutical compositions may be formulated in a “therapeutically effective amount” or a “prophylactically effective amount”. A “therapeutically effective amount” refers to an amount effective, at dosages and for periods of time necessary, to achieve the desired therapeutic result. A therapeutically effective amount of a recombinant vector may vary depending on the condition to be treated, the severity and course of the condition, the mode of administration, whether the antibody or agent is administered for preventive or therapeutic purposes, the bioavailability of the particular agent(s), the ability of the bispecific antagonist to elicit a desired response in the individual, previous therapy, the age, weight and sex of the patient, the patient's clinical history and response to the antibody, the type of the bispecific antagonist used, discretion of the attending physician, etc. A therapeutically effective amount is also one in which any toxic or detrimental effect of the recombinant vector is outweighed by the therapeutically beneficial effects. A “prophylactically effective amount” refers to an amount effective, at dosages and for periods of time necessary, to achieve the desired prophylactic result.

Preferably, the polypeptide domains in the bispecific antagonist are derived from the same host in which they are to be administered in order to reduce inflammatory responses against the administered therapeutic agents.

The bispecific antagonist is suitably administered to the patient at one time or over a series of treatments and may be administered to the patient at any time from diagnosis onwards. The bispecific antagonist may be administered as the sole treatment or in conjunction with other drugs or therapies useful in treating the condition in question.

For treatment of eye disorders, a therapeutically effective amount or prophylactically effective amount of the bispecific antagonist can be administered by intravitreal injection in a volume of about 10 μl to about 100 μl per injection, about 10 μl to about 90 μl per injection, about 10 μl to about 80 μl per injection, about 10 μl to about 70 μl per injection, about 10 μl to about 60 μl per injection, about 10 μl to about 50 μl per injection, about 10 μl to about 40 μl per injection, about 10 μl to about 30 μl per injection, about 10 μl to about 20 μl per injection, about 20 μl to about 100 μl per injection, about 20 μl to about 90 μl per injection, about 20 μl to about 80 μl per injection, about 20 μl to about 70 μl per injection, about 20 μl to about 60 μl per injection, about 20 μl to about 50 μl per injection, about 20 μl to about 40 μl per injection, about 20 μl to about 30 μl per injection, about 30 μl to about 100 μl per injection, about 30 μl to about 90 μl per injection, about 30 μl to about 80 μl per injection, about 30 μl to about 70 μl per injection, about 30 μl to about 60 μl per injection, about 30 μl to about 50 μl per injection, about 30 μl to about 40 μl per injection, about 40 μl to about 100 μl per injection, about 40 μl to about 90 μl per injection, about 40 μl to about 80 μl per injection, about 40 μl to about 70 μl per injection, about 40 μl to about 60 μl per injection, about 40 μl to about 50 μl per injection, about 50 μl to about 100 μl per injection, about 50 μl to about 90 μl per injection, about 50 μl to about 80 μl per injection, about 50 μl to about 70 μl per injection, about 50 μl to about 60 μl per injection, about 60 μl to about 100 μl per injection, about 60 μl to about 90 μl per injection, about 60 μl to about 70 μl per injection, about 70 μl to about 100 μl per injection, about 80 μl to about 90 μl per injection, about 90 μl to about 100 μl per injection, or any ul amount per injection corresponding to the foregoing integers.

A therapeutically effective or prophylactically effective amount of each bispecific antagonist may be administered intravitreally in a range from about 100 μg to about 1,000 μg per injection, about 100 μg to about 900 μg per injection, about 100 μg to about 800 μg per injection, about 100 μg to about 700 μg per injection, about 100 μg to about 600 μg per injection, about 100 μg to about 500 μg per injection, about 100 μg to about 400 μg per injection, about 100 μg to about 300 μg per injection, about 100 μg to about 200 μg per injection, about 200 μg to about 1,000 μg per injection, about 200 μg to about 900 μg per injection, about 200 μg to about 800 μg per injection, about 200 μg to about 700 μg per injection, about 200 μg to about 600 μg per injection, about 200 μg to about 500 μg per injection, about 200 μg to about 400 μg per injection, about 200 μg to about 300 μg per injection, about 300 μg to about 1,000 μg per injection, about 300 μg to about 900 μg per injection, about 300 μg to about 800 μg per injection, about 300 μg to about 700 μg per injection, about 300 μg to about 600 μg per injection, about 300 μg to about 500 μg per injection, about 300 μg to about 400 μg per injection, about 400 μg to about 1,000 μg per injection, about 400 μg to about 900 μg per injection, about 400 μg to about 800 μg per injection, about 400 μg to about 700 μg per injection, about 400 μg to about 600 μg per injection, about 400 μg to about 500 μg per injection, about 500 μg to about 1,000 μg per injection, about 500 μg to about 900 μg per injection, about 500 μg to about 800 μg per injection, about 500 μg to about 700 μg per injection, about 500 μg to about 600 μg per injection, about 600 μg to about 1,000 μg per injection, about 600 μg to about 900 μg per injection, about 600 μg to about 800 μg per injection, about 600 μg to about 700 μg per injection, about 700 μg to about 1,000 μg per injection, about 700 μg to about 900 μg per injection, about 700 μg to about 800 μg per injection, about 800 μg to about 1,000 μg per injection, about 800 μg to about 900 μg per injection, about 900 μg to about 1,000 μg per injection, or any μg amount per injection corresponding to the foregoing integers.

Further, each bispecific antagonist may be administered intravitreally once a week, once every two weeks, once every three weeks, once a month, once every two months, once every 3 months, once every 4 months, once every 6 months, once every 8 months, once a year, or any combination thereof.

In certain embodiments, the coding sequence for a bispecific antagonist is incorporated into a suitable expression vector (e.g., viral or non-viral vector) for expressing an effective amount of the bispecific antagonist in a patient with an angiogenic eye disorder.

In certain embodiments comprising administration of e.g., one or more recombinant AAV (rAAV) viruses. The rAAV virus is formed from rAAV vector. As used herein, a “rAAV vector” refers to a vector derived from an adeno-associated virus serotype, including without limitation, AAV-1, AAV-2, AAV-3, AAV-4, AAV-5, AAV-6, AAV-7, AAV-8, AAV-9 etc. Typically, an AAV vector has one or more AAV wild-type genes deleted in whole or part, usually the rep and/or cap genes, but retains functional flanking ITR sequences. Functional ITR sequences are necessary for the rescue, replication and packaging of the AAV virion. Thus, an AAV vector includes at least those sequences required in cis for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging.

The “rAAV virus” or “rAAV virion” is as an infectious, replication-defective virus composed of an AAV protein shell encapsulating a heterologous nucleotide sequence of interest that is flanked on both sides by AAV ITRs. The rAAV virion is produced in a suitable host cell comprising the rAAV vector, AAV helper functions, and accessory functions. In this manner, the host cell is rendered capable of encoding AAV polypeptides that are required for packaging the AAV vector (containing a recombinant nucleotide sequence of interest) into infectious recombinant virion particles for subsequent gene delivery. Generally, one or more helper functions (typically AAV rep and/or cap) are provided in trans to facilitate production (or packaging) of the rAAV vector to produce rAAV viruses for gene delivery of the multispecific antibodies of the present application.

In some embodiments, the the pharmaceutical composition comprises a rAAV vector or rAAV virus capable of expressing a bispecific antibody comprising: (1) a trebanib peptide; and (2) an aflibercept domain. In specific embodiments, the a rAAV vector or rAAV virus is capable of expressing a bispecific antibody selected from the group consisting of Bi-ZT-1, Bi-ZT-2, Bi-ZT-3, Bi-ZT-4, Bi-ZT5, A2Z-d, ZA2-s, ZA2-d, ZA2-Is, ZA2-Id-1, and ZA2-Id-2.

In some embodiments, the pharmaceutical composition comprises a rAAV vector or rAAV virus capable of expressing a bispecific antibody comprising: (1) a wild-type or mutant bevacizumab (Avastin®) domain; and (2) a trebanib peptide. In specific embodiments, the rAAV vector or rAAV virus is capable of expressing a bispecific antibody selected from the group consisting of Bi-BT-1, Bi-BT-2, AsA2d, and AsA2Id.

In some embodiments, the pharmaceutical composition comprises a rAAV vector or rAAV virus capable of expressing a trispecific antibody comprising: (1) a wild-type or mutant bevacizumab (Avastin®) N-terminal domain; (2) a wild-type or mutant TGF-β1 RII-ECD C-terminal domain; and (3) a trebananib peptide domain inserted within an IgG CH3 region. In specific embodiments, the rAAV vector or rAAV virus is capable of expressing a trispecific antibody selected from the group consisting of ABT, ABT-1U, ABmT-1U, and AmBT.

In some embodiments, the pharmaceutical composition comprises a rAAV in an amount comprising at least 10¹⁰, at least 10¹¹, at least 10¹², at least 10¹³, or at least 10¹⁴ genome copies (GC) or recombinant viral particles per kg, or any range thereof. In certain embodiments, pharmaceutical composition comprises an effective amount of the recombinant virus, such as rAAV, in an amount comprising at least 10¹⁰, at least 10¹¹, at least 10¹², at least 10¹³, at least 10¹⁴, at least 10¹⁵ genome copies or recombinant viral particles genome copies per subject, or any range thereof.

Dosages can be tested in several art-accepted animal models suitable for any particular eye disease.

Delivery methodologies may also include the use of polycationic condensed DNA linked or unlinked to killed viruses, ligand linked DNA, liposomes, eukaryotic cell delivery vehicles cells, deposition of photopolymerized hydrogel materials, use of a handheld gene transfer particle gun, ionizing radiation, nucleic charge neutralization or fusion with cell membranes, particle mediated gene transfer and the like.

### V. Methods of Making the Multispecific Antagonists

### A. Nucleic Acids and Host Cells for Antagonist Expression

In one aspect, the present application provides nucleic acids encoding the multispecific antagonist of the present application, and expression vectors comprising such nucleic acids. In some embodiments, nucleic acids encodes an HCVR and/or LCVR fragment of an antibody or fragment in accordance with the embodiments described herein, or any of the other antibodies and antibody fragments described herein.

DNA encoding an antigen binding site in a monoclonal antibody can be isolated and sequenced from the hybridoma cells using conventional procedures (e.g., by using oligonucleotide probes that are capable of binding specifically to genes encoding the heavy and light chains of the monoclonal antibodies). Alternatively, amino acid sequences from immunoglobulins of interest may be determined by direct protein sequencing, and suitable encoding nucleotide sequences can be designed according to a universal codon table. In other cases, nucleotide and amino acid sequences of antigen binding sites or other immunoglobulin sequences, including constant regions, hinge regions and the like may be obtained from published sources well known in the art.

Expression vectors may be used to synthesize the bispecific antagonists of the present disclosure in cultured cells in vitro or they may be directly administered to a patient to express the bispecific antagonist in vivo or ex vivo. As used herein, an “expression vector” refers to a viral or non-viral vector comprising a polynucleotide encoding one or more polypeptide chains corresponding to a bispecific antagonist of the present disclosure in a form suitable for expression from the polynucleotide(s) in a host cell for antagonist/antibody preparation purposes or for direct administration as a therapeutic agent.

A nucleic acid sequence is “operably linked” to another nucleic acid sequence when the former is placed into a functional relationship with the latter. For example, a DNA for a presequence or signal peptide is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a signal peptide, contiguous and in reading phase. However, enhancers do not have to be contiguous. Linking is accomplished by ligation at convenient restriction sites. If such sites do not exist, synthetic oligonucleotide adaptors or linkers may be used in accordance with conventional practice.

Nucleic acid sequences for expressing the bispecific antagonists typically include an amino terminal signal peptide sequence, which is removed from the mature protein. Since the signal peptide sequences can affect the levels of expression, the polynucleotides may encode any one of a variety of different N-terminal signal peptide sequences. It will be appreciated by those skilled in the art that the design of the expression vector can depend on such factors as the choice of the host cell to be transformed, the level of expression of protein desired, and the like.

The above described “regulatory sequences” refer to DNA sequences necessary for the expression of an operably linked coding sequence in one or more host organisms. The term “regulatory sequences” is intended to include promoters, enhancers and other expression control elements (e.g., polyadenylation signals). Regulatory sequences include those which direct constitutive expression of a nucleotide sequence in many types of host cells or those which direct expression of the nucleotide sequence only in certain host cells (e.g., tissue-specific regulatory sequences). Expression vectors generally contain sequences for transcriptional termination, and may additionally contain one or more elements positively affecting mRNA stability.

The expression vector contains one or more transcriptional regulatory elements, including promoters and/or enhancers, for directing expression of the bispecific antagonist. A promoter comprises a DNA sequence that functions to initiate transcription from a relatively fixed location in regard to the transcription start site. A promoter contains core elements required for basic interaction of RNA polymerase and transcription factors, and may operate in conjunction with other upstream elements and response elements.

As used herein, the term “promoter” is to be taken in its broadest context and includes transcriptional regulatory elements (TREs) from genomic genes or chimeric TREs therefrom, including the TATA box or initiator element for accurate transcription initiation, with or without additional TREs (i.e., upstream activating sequences, transcription factor binding sites, enhancers, and silencers) which regulate activation or repression of genes operably linked thereto in response to developmental and/or external stimuli, and trans-acting regulatory proteins or nucleic acids. A promoter may contain a genomic fragment or it may contain a chimera of one or more TREs combined together.

Preferred promoters are those capable of directing high-level expression in a target cell of interest. The promoters may include constitutive promoters (e.g., HCMV, SV40, elongation factor-1α (EF-1α)) or those exhibiting preferential expression in a particular cell type of interest. Enhancers generally refer to DNA sequences that function away from the transcription start site and can be either 5′ or 3′ to the transcription unit. Furthermore, enhancers can be within an intron as well as within the coding sequence. They are usually between 10 and 300 bp in length, and they function in cis. Enhancers function to increase and/or regulate transcription from nearby promoters. Preferred enhancers are those directing high-level expression in the antibody producing cell. Cell or tissue-specific transcriptional regulatory elements (TREs) can be incorporated into expression vectors to restrict expression to desired cell types. Pol III promoters (H1 or U6) are particularly useful for expressing shRNAs from which siRNAs are expressed. An expression vector may be designed to facilitate expression of the bispecific antagonist in one or more cell types.

In certain embodiments, one or more expression vectors may be engineered to express both the bispecific antagonist and one or more siRNAs targeting the VEGF pathway, Tie2 pathway, or other pathway associated with the eye disease for treatment.

An siRNA is a double-stranded RNA that can be engineered to induce sequence-specific post-transcriptional gene silencing of mRNAs. Synthetically produced siRNAs structurally mimic the types of siRNAs normally processed in cells by the enzyme Dicer. When expressed from an expression vector, the expression vector is engineered to transcribe a short double-stranded hairpin-like RNA (shRNA) that is processed into a targeted siRNA inside the cell. Synthetic siRNAs and shRNAs may be designed using well known algorithms and synthesized using a conventional DNA/RNA synthesizer.

To co-express the individual chains of the bispecific antagonist, a suitable splice donor and splice acceptor sequences may be incorporated for expressing both products. Alternatively, an internal ribosome binding sequence (IRES) or a 2A peptide sequence, may be employed for expressing multiple products from one promoter. An IRES provides a structure to which the ribosome can bind that does not need to be at the 5′ end of the mRNA. It can therefore direct a ribosome to initiate translation at a second initiation codon within a mRNA, allowing more than one polypeptide to be produced from a single mRNA. A 2A peptide contains short sequences mediating co-translational self-cleavage of the peptides upstream and downstream from the 2A site, allowing production of two different proteins from a single transcript in equimolar amounts. CHYSEL is a non-limiting example of a 2A peptide, which causes a translating eukaryotic ribosome to release the growing polypeptide chain that it is synthesizing without dissociating from the mRNA. The ribosome continues translating, thereby producing a second polypeptide.

An expression vector may comprise a viral vector or a non-viral vector. A viral vectors may be derived from an adeno-associated virus (AAV), adenovirus, herpesvirus, vaccinia virus, poliovirus, poxvirus, a retrovirus (including a lentivirus, such as HIV-1 and HIV-2), Sindbis and other RNA viruses, alphavirus, astrovirus, coronavirus, orthomyxovirus, papovavirus, paramyxovirus, parvovirus, picornavirus, togaviruses and the like. A non-viral vector is simply a “naked” expression vector that is not packaged with virally derived components (e.g., capsids and/or envelopes).

In certain cases, these vectors may be engineered to target certain diseases or cell populations by using the targeting characteristics inherent to the virus vector or engineered into the virus vector. Specific cells may be “targeted” for delivery of polynucleotides, as well as expression. Thus, the term “targeting”, in this case, may be based on the use of endogenous or heterologous binding agents in the form of capsids, envelope proteins, antibodies for delivery to specific cells, the use of tissue-specific regulatory elements for restricting expression to specific subset(s) of cells, or both.

In some embodiments, expression of the antibody chains is under the control of the regulatory element such as a tissue specific or ubiquitous promoter. In some embodiments, a ubiquitous promoter such as a CMV promoter, CMV-chicken beta-actin hybrid (CAG) promoter, or a tissue specific promoter to control the expression of a particular antibody heavy or light chain or single-chain derivative therefrom.

Non-viral expression vectors can be utilized for non-viral gene transfer, either by direct injection of naked DNA or by encapsulating the bispecific antagonist-encoding polynucleotides in liposomes, microparticles, microcapsules, virus-like particles, or erythrocyte ghosts. Such compositions can be further linked by chemical conjugation to targeting domains to facilitate targeted delivery and/or entry of nucleic acids into desired cells of interest. In addition, plasmid vectors may be incubated with synthetic gene transfer molecules such as polymeric DNA-binding cations like polylysine, protamine, and albumin, and chemically linked to cell targeting ligands such as asialoorosomucoid, insulin, galactose, lactose or transferrin.

Alternatively, naked DNA may be employed. Uptake efficiency of naked DNA may be improved by compaction or by using biodegradable latex beads. Such delivery may be improved further by treating the beads to increase hydrophobicity and thereby facilitate disruption of the endosome and release of the DNA into the cytoplasm.

### B. Methods for Producing Multispecific Antibodies

In one aspect, the present application provides host cells transformed with the fusion protein-encoded nucleic acids of the present application, including expression vectors encoding the bispecific antagonists of the present application. The host cells can be any bacterial or eukaryotic cell capable of expressing the fusion protein-encoded nucleic acids encoding nucleic acids or expression vectors or any of the other co-administered antibodies or antagonists described herein.

In another aspect, a method of producing a bispecific antagonist comprises culturing a host cell transformed with one or more fusion protein-encoding nucleic acids or expression vectors under conditions that allows production of the antagonist, and purifying the antagonist from the cell.

In a further aspect, the present application provides a method for producing an antagonist comprising culturing a cell transiently or stably expressing one or more constructs encoding one or more polypeptide chains in the antagonist; and purifying the antagonist from the cultured cells. Any cell capable of producing a functional antagonist may be used. In preferred embodiments, the antagonist-expressing cell is of eukaryotic or mammalian origin, preferably a human cell. Cells from various tissue cell types may be used to express the antagonist. In other embodiments, the cell is a yeast cell, an insect cell or a bacterial cell. Preferably, the antagonist-producing cell is stably transformed with a vector expressing the antagonist.

One or more expression vectors encoding an antagonist fusion protein subunit or an antibody antagonist heavy or light chain can be introduced into a cell by any conventional method, such as by naked DNA technique, cationic lipid-mediated transfection, polymer-mediated transfection, peptide-mediated transfection, virus-mediated infection, physical or chemical agents or treatments, electroporation, etc. In addition, cells may be transfected with one or more expression vectors for expressing a bispecific antagonist along with a selectable marker facilitating selection of stably transformed clones expressing the bispecific antagonist. The antagonists produced by such cells may be collected and/or purified according to techniques known in the art, such as by centrifugation, chromatography, etc.

Examples of suitable selectable markers for mammalian cells include dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, mycophenolic acid, or hygromycin. The three examples employ bacterial genes under eukaryotic control to convey resistance to the appropriate drug G418 or neomycin (geneticin), xgpt (mycophenolic acid) or hygromycin, respectively. Others include the neomycin analog G418 and puromycin.

Exemplary antagonist-expressing cells include human Jurkat, human embryonic kidney (HEK) 293, Chinese hamster ovary (CHO) cells, mouse WEHI fibrosarcoma cells, as well as unicellular protozoan species, such as Leishmania tarentolae. In addition, stably transformed, antagonist producing cell lines may be produced using primary cells immortalized with c-myc or other immortalizing agents.

In one embodiment, the cell line comprises a stably transformed Leishmania cell line, such as Leishmania tarentolae. Leishmania are known to provide a robust, fast-growing unicellular host for high level expression of eukaryotic proteins exhibiting mammalian-type glycosylation patterns. A commercially available Leishmania eukaryotic expression kit is available (Jena Bioscience GmbH, Jena, Germany).

In some embodiments, the cell line expresses at least 1 mg, at least 2 mg, at least 5 mg, at least 10 mg, at least 20 mg, at least 50 mg, at least 100 mg, at least 200 mg, at least 300 mg, at least 400 mg, or at least 500 mg of the antibody/liter of culture.

The antagonists of the present application may be isolated from antagonist expressing cells following culture and maintenance in any appropriate culture medium, such as RPMI, DMEM, and AIM V®. The antagonists can be purified using conventional protein purification methodologies (e.g., affinity purification, chromatography, etc.), including the use of Protein-A or Protein-G immunoaffinity purification. In some embodiments, antagonists are engineered for secretion into culture supernatants for isolation therefrom.

The present application is further illustrated by the following examples which should not be construed as limiting. The contents of all references, patents and published patent applications cited throughout this application, as well as the Figures and Tables are incorporated herein by reference.

### C. Homodimers and Heterodimers

One of the challenges for efficiently producing bispecific antagonists comprising Ig heavy and light chains concerns mispairing of the heavy and light chains, when co-expressing chains of different binding specificities. Table 1 lists several amino acid substitution options for overcoming mispairing between heavy chains of different binding specificities, which “enforce” or preferentially promote correct association between desired heavy chains. Any approach to prevent or reduce mispairing between heavy chains may be used to make the bispecific antagonists according to the present disclosure.

The “knobs-into-hole” (KiH) approach relies on modifications of the interface between the two CH3 domains where most interactions occur. Typically, a bulky residue is introduced into the CH3 domain of one antibody heavy chain and acts similarly to a key. In the other heavy chain, a “hole” is formed that is able to accommodate this bulky residue, mimicking a lock. The resulting heterodimeric Fc-part can be further stabilized by artificial disulfide bridges.

An alternative approach is based on charged residues with ionic interactions or steric complementarity. This includes altering the charge polarity in the CH3 interface so that co-expression of electrostatically matched Fc domains support favorable attractive interactions and heterodimer formation while retaining the hydrophobic core, whereas unfavorable repulsive charge interactions suppress homodimerization. See Table 1. The amino acid numbering in Table 1 follows the Kabat numbering scheme and can be applied to heavy chain amino acid sequences of the antibodies described herein.

In a further approach, leucine zipper (LZ) domains may be incorporated into a protein scaffold. A leucine zipper is a common three-dimensional structural motif in proteins, typically as part of a DNA-binding domain in various transcription factors. A single LZ typically contains 4-5 leucine residues at approximately 7-residue intervals, which forms an amphipathic alpha helix with a hydrophobic region running along one side. In a particular embodiment, a heterodimeric protein scaffold comprises a LZ from the c-jun transcription factor associated with a LZ from the c-fos transcription factor. Although c-jun is known to form jun-jun homodimers and c-fos does not form homodimers, the formation of jun-fos heterodimers is greatly favored over jun-jun homodimers.

A leucine zipper domain may be incorporated in place of CH2-CH3 sequences in the protein scaffold or it may be placed at the carboxy terminal end of the two heavy chains in a bispecific antibody antagonist. In the case of the latter, a furin cleavage site may be introduced between the carboxy terminal end of CH3 and the amino terminal end of the leucine zipper. This can facilitate furin-mediated cleavage of the leucine zipper following the heterodimerization step when co-expressing the heavy and light chains of the bispecific in an appropriate mammalian cell expression system (see Wranik et al., J. Biol. Chem., 287(5):43331-43339, 2012).

TABLE 1

| Type | HC1 | HC2 |
|---|---|---|
| Knobs-into-holes | Y349C, T366S, | S354C, T366W |
| | L368A, Y407V | |
| Ionic, electrostatic | S183E, E356K, | S183K, K370E, |
| | E357K, D399K | K409D, K439E |
| Ionic, electrostatic | K392D, K409D | E356K, D399K |
| HA-TF substitutions | S364H, F405A | Y349T, T394F |
| HF-TA substitutions | S364H, T394F | Y349T, F405A |
| Leucine zipper | human c-Jun | human c-fos |
| heterodimer | leucine zipper | leucine zipper |

The amino acid numbering in Table 1 follows the Kabat numbering scheme and can be applied to heavy chain amino acid sequences of the antibodies described herein. The mutations described in Table 1 may be applied to the sequence (published or otherwise) of any immunoglobulin IgG1 heavy chain, as well as other immunoglobulin classes, and subclasses (or isotypes) therein.

When co-expressing heavy and light chains of bispecific antibody antagonists, the light chains of one binding specificity can also mispair with heavy chains of a different binding specificity. Therefore, in certain embodiments, portions of the heavy chain, light chain or both may be modified relative to the “wild-type” antibody chains from which they are derived to prevent or reduce mispairing of both heavy chain constant regions to one another, as well mispairing of light chain constant regions to their heavy chain counterparts.

The light chain mispairing problem can be addressed in several ways. In some embodiments, sterically complementary mutations and/or disulfide bridges may be incorporated into the two VL/VH interfaces. In other embodiments, mutations can be incorporated based on ionic or electrostatic interactions. In some embodiments, light chain mispairing may be prevented or reduced by employing a first arm with an S183E mutation in the CH1 domain of the heavy chain and an S176K mutation in the CL domain of the light chain. A second arm may include an S183K mutation in the in the CH1 domain of the heavy chain and an S176E mutation in the CL domain of the light chain. In other embodiments, a “CrossMab” approach is employed, where one arm in the bispecific antibody (e.g., Fab) is left untouched, but in the other arm containing the other binding specificity, one or more domains in the light chain are swapped with one or more domains in the heavy chain at the heavy chain: light chain interface.

Methods, immunoglobulin domain sequences, including specific mutations for preventing mispairing of heavy and light chains as disclosed above are further described in U.S. Patent Application Publication Nos. 2014/0243505, 2013/0022601.

### D. Conjugates

In certain embodiments, the bispecific antagonists of the present application are chemically conjugated to one or more peptides and/or small molecule drugs. The peptides or small molecule drug can be the same or different. The peptides or small molecule drugs can be attached, for example to reduced SH groups and/or to carbohydrate side chains. Methods for making covalent or non-covalent conjugates of peptides or small molecule drugs with antibodies are known in the art and any such known method may be utilized.

In some embodiments the peptide or small molecule drug is attached to the hinge region of a reduced antibody component via disulfide bond formation. Alternatively, such agents can be attached using a heterobifunctional cross-linkers, such as N-succinyl 3-(2-pyridyldithio)propionate (SPDP). General techniques for such conjugation are well-known in the art. In some embodiments, the peptide or small molecule drug is conjugated via a carbohydrate moiety in the Fc region of the antibody. The carbohydrate group can be used to increase the loading of the same agent that is bound to a thiol group, or the carbohydrate moiety can be used to bind a different therapeutic or diagnostic agent. Methods for conjugating peptide inhibitors or small molecule drugs to antibodies via antibody carbohydrate moieties are well-known to those of skill in the art. For example, in one embodiment, the method involves reacting an antibody component having an oxidized carbohydrate portion with a carrier polymer that has at least one free amine function. This reaction results in an initial Schiff base (imine) linkage, which can be stabilized by reduction to a secondary amine to form the final conjugate. Exemplary methods for conjugating small molecule drugs and peptides to antibodies are described in U.S. Patent Application Publication No. 2014/0356385.

Preferably, the bispecific antagonists in the present disclosure retain certain desirable characteristics and pharmacokinetic properties of antibodies, including a desirable in vitro and in vivo stability (e.g., lone half-life and shelf-life stability), efficient delivery into desired target cells, increased affinity for binding partners, desirable antibody-dependent cell-mediated cytotoxicity and complement-dependent cytotoxicity, and reduced renal clearance or excretion. Accordingly, careful attention to size and need for particular constant region effector functions may be considered in the design of the bispecific antagonists.

The bispecific antagonists of the present application may range in size from 50 kD to 300 kD, from 50 kD to 250 kD, from 60 kD to 250 kD, from 80 kD to 250 kD, from 100 kD to 250 kD, from 125 kD to 250 kD, from 150 kD to 250 kD, from 60 kD to 225 kD, from 75 kD to 225 kD, from 100 kD to 225 kD, from 125 kD to 225 kD, from 150 kD to 225 kD, from 60 kD to 200 kD, from 75 kD to 200 kD, from 100 kD to 125 kD to 200 kD, from 150 kD to 200 kD, from 60 kD to 150 kD, from 75 kD to 150 kD, from 100 kD to 150 kD, from 60 kD to 125 kD, from 75 kD to 125 kD, from 75 kD to 100 kD, or any range encompassed by any combination of whole numbers listed in the above cited ranges or any ranges specified by any combination of whole numbers between any of the above cited ranges.

### EXAMPLES

### Example 1: Bispecific Antagonists Containing VEGF Binding Domains and Ang2-Specific Peptides

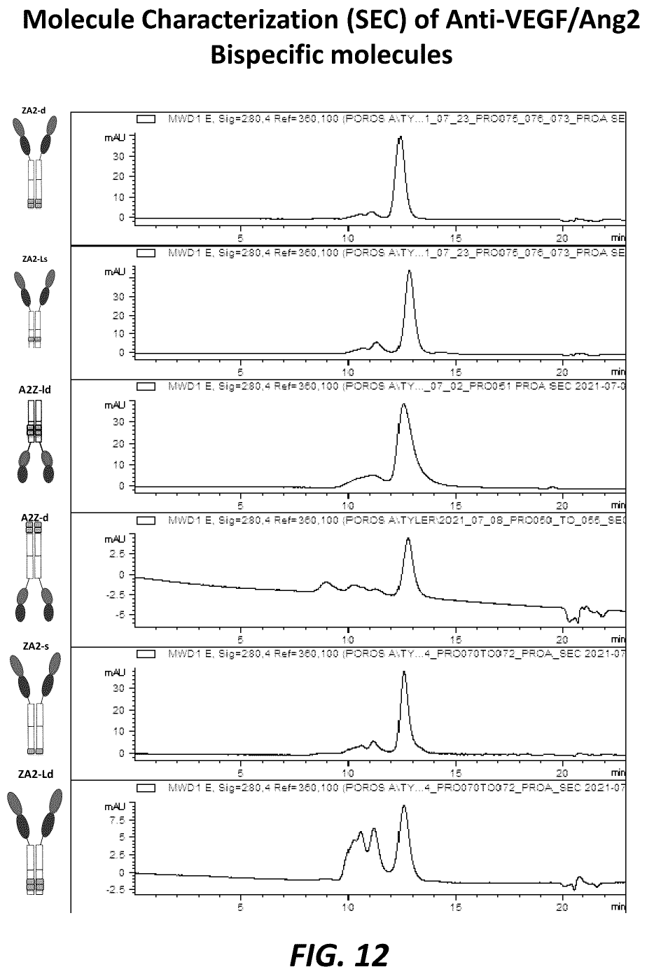

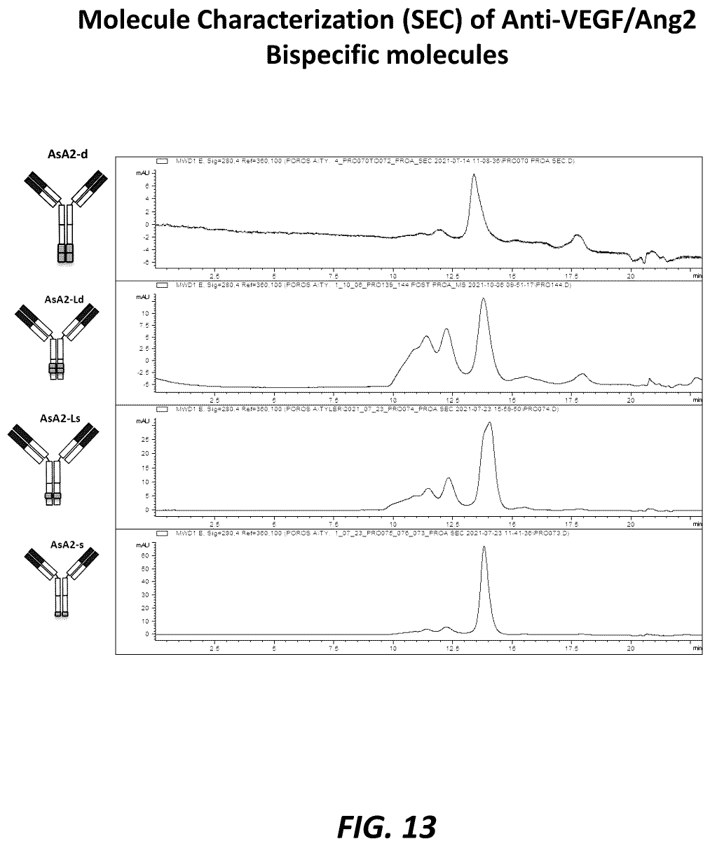

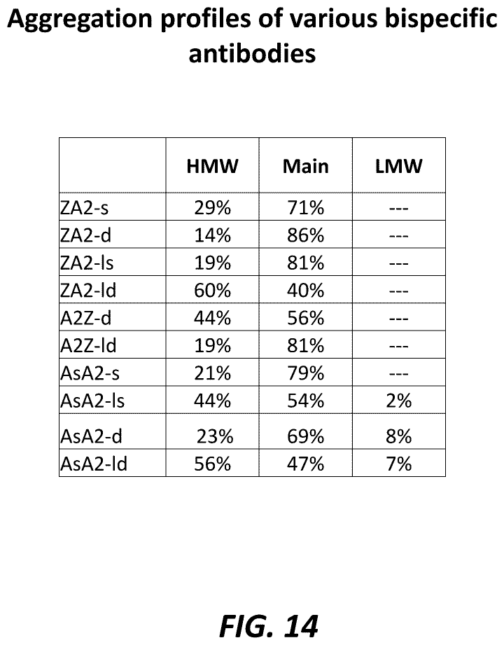

DNA expression vectors encoding the bispecific antagonists of FIG. 1 were constructed using standard molecular cloning techniques. The bisepecific constructs were transiently expressed in HEK293 cells, affinity purified by HPLC-Protein A and subjected to HPLC analysis for SEC profiles. Briefly, 5-10 μgs of purified proteins were injected to a TOSOH UP-SW3000 size exclusion column, and the UV peaks were integrated to calculate the percent distribution of high molecular weight, main peak, and low molecular weight species. FIGS. 12 and 13 show molecule characterization (SEC) of anti-VEGF/Ang2 bispecific constructs of FIG. 1. The aggregation profiles of the bispecific constructs are summarized in FIG. 14. These constructs may be used for treating wet AMD and cancers.

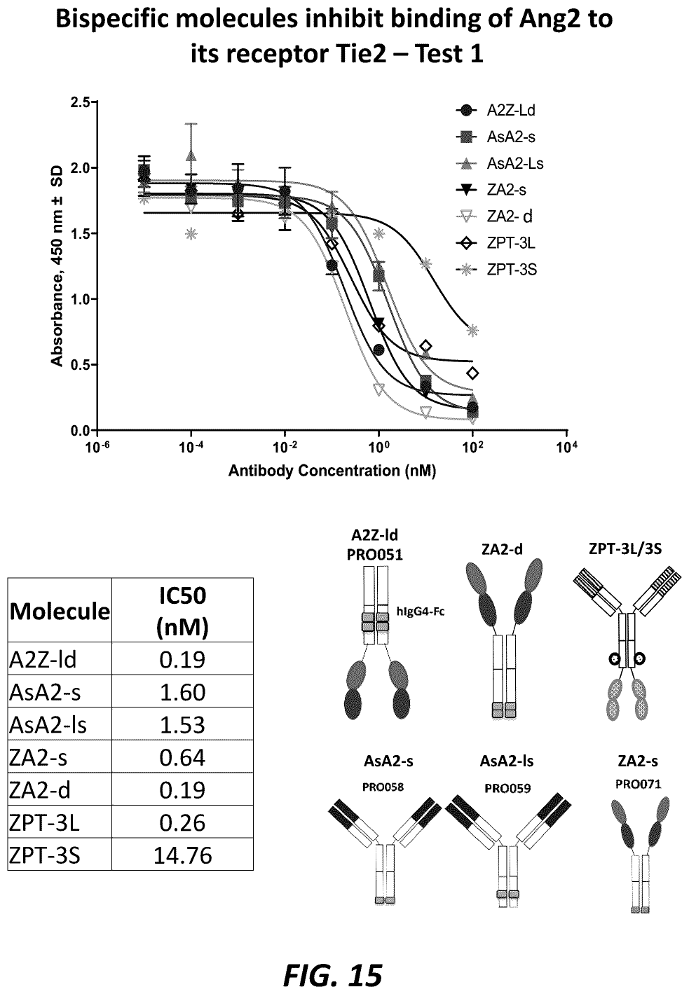

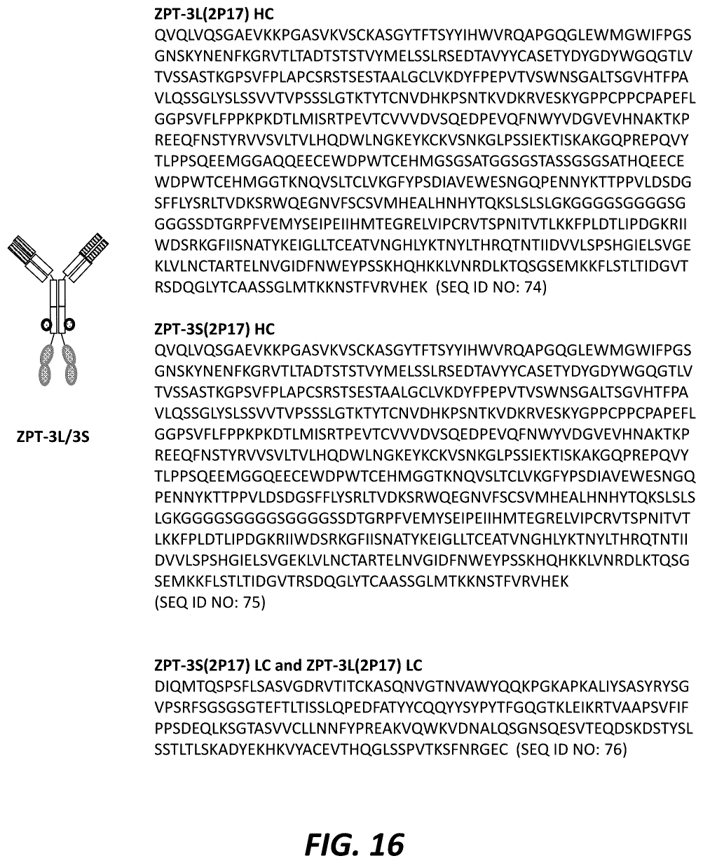

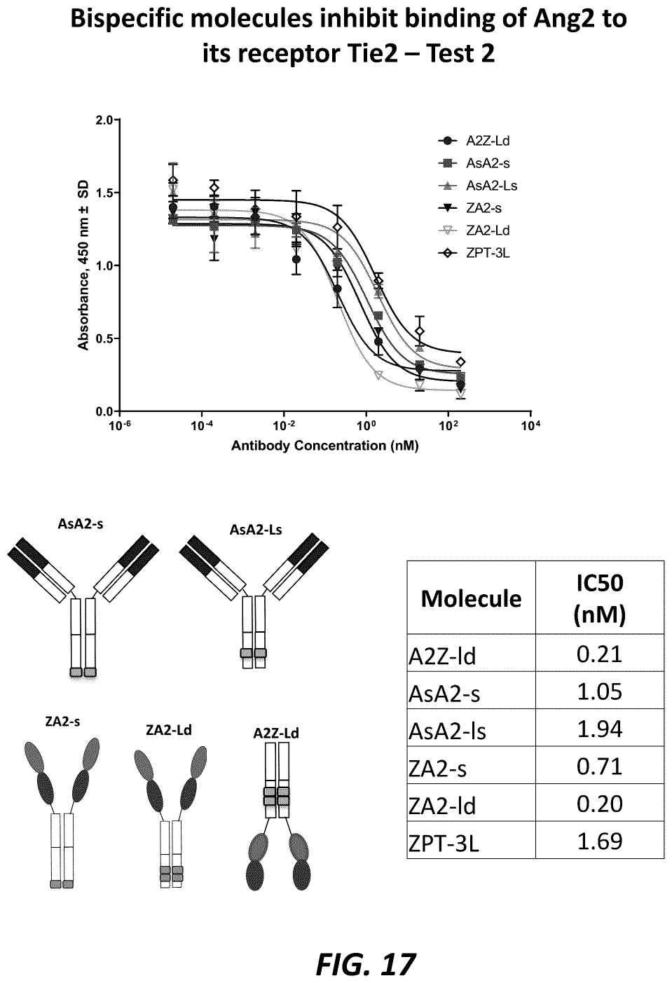

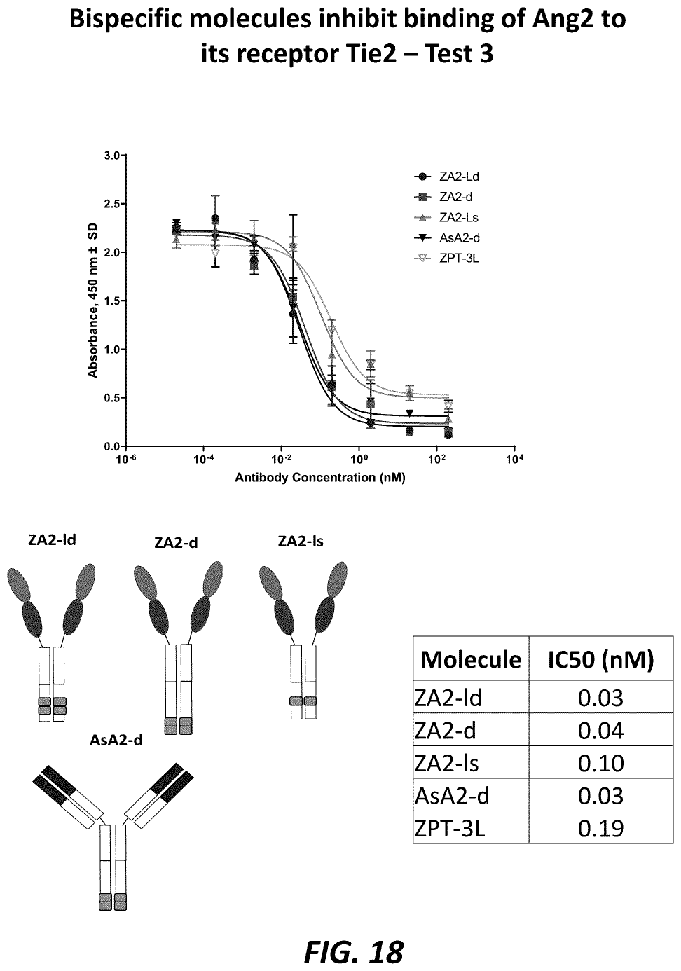

FIGS. 15, 17 and 18 are diagrams showing that exemplary bispecific molecules inhibit binding of Ang2 to its receptor Tie2. Compared to ZPT molecules (ZPT-3L/3S) which contain the long or short version of the trebananib peptide (AMG386) that inhibit both Ang2 and Ang1, A2 peptide is more potent and specifically inhibit Ang2 activity.

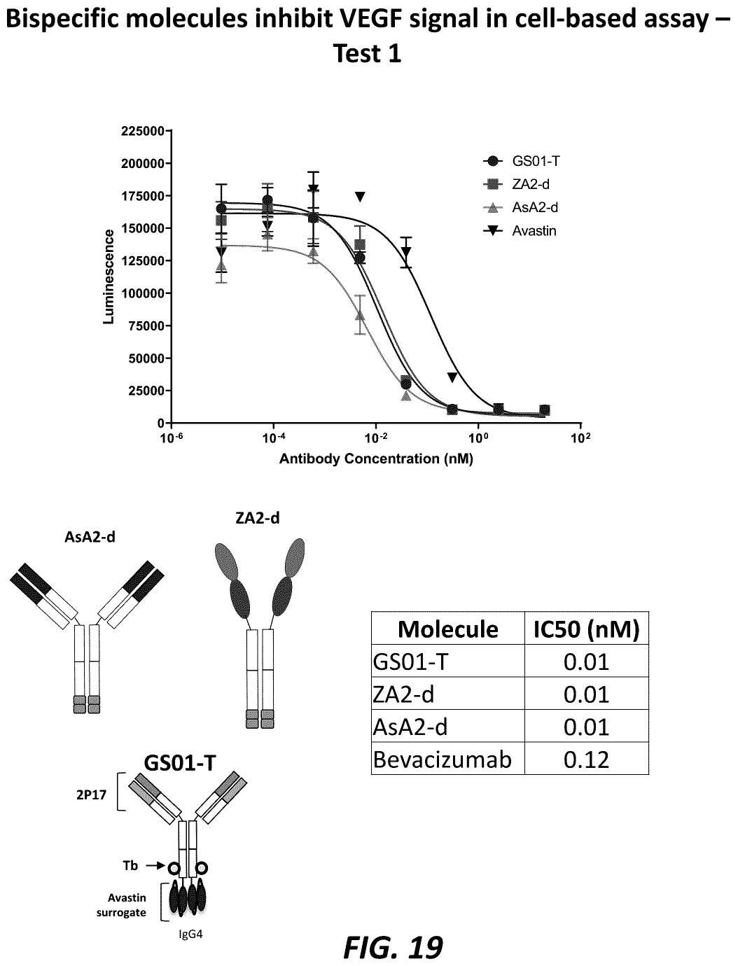

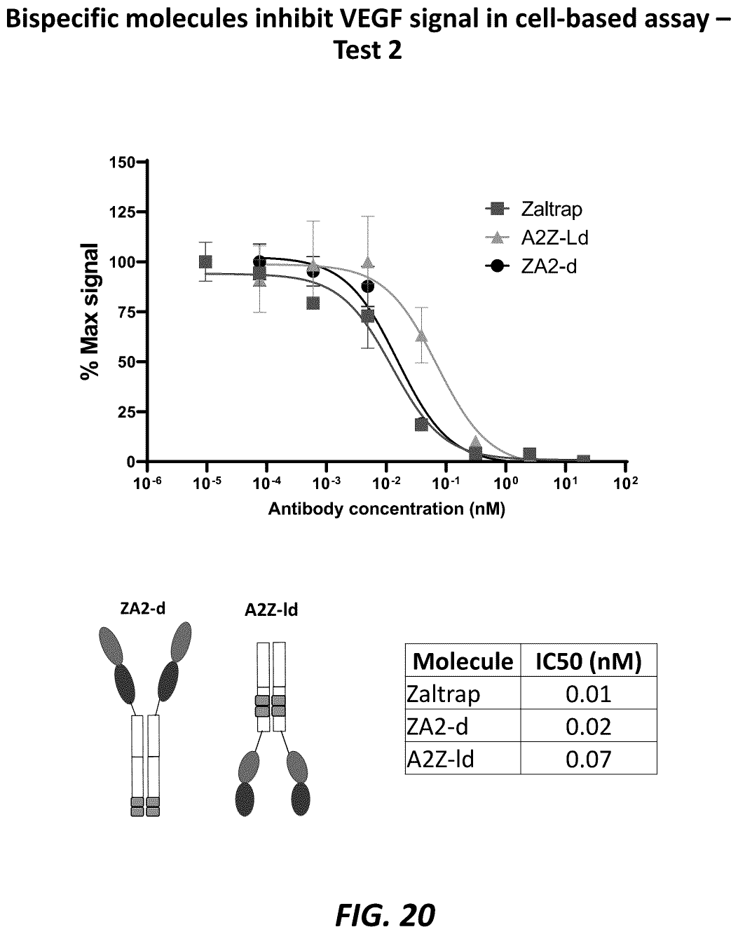

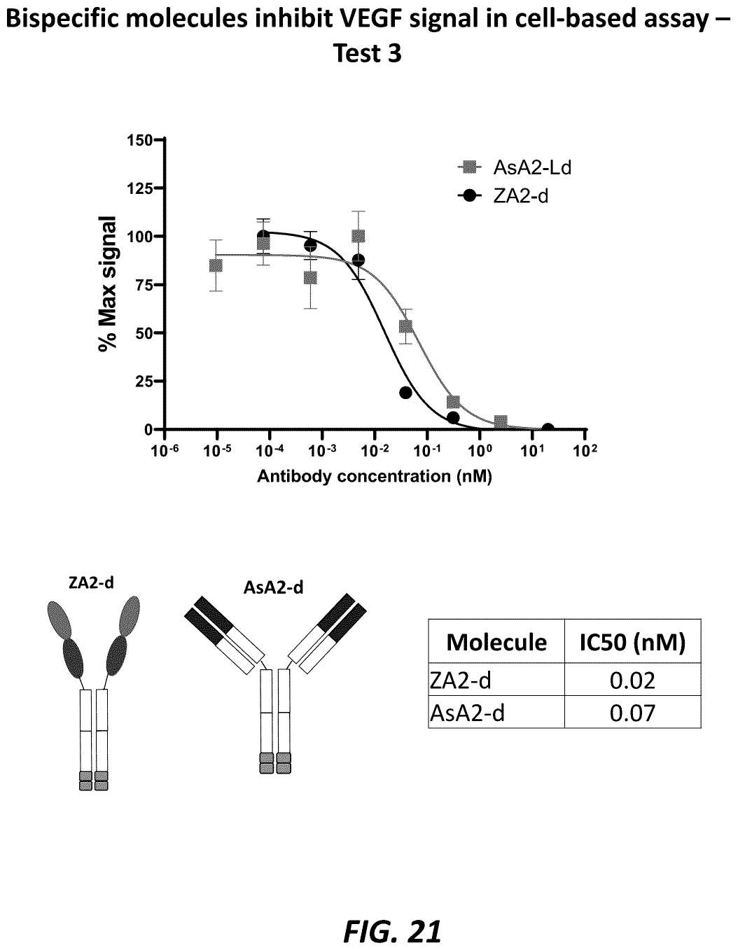

FIGS. 19-21 are diagrams showing that the exemplary bispecific molecules of the present application inhibit VEGF signal in cell-based assay. Bispecific constructs ZA2-d and A2Z-ld are very potent at inhibiting VEGF signals.

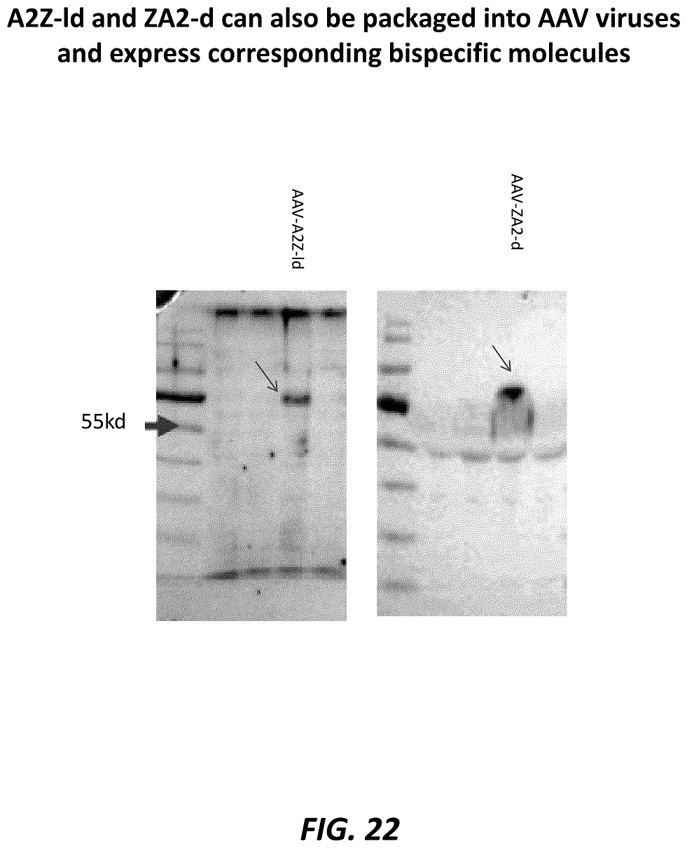

FIG. 22 is a Western blot showing that A2Z-ld and ZA2-d can also be packaged into AAV viruses and express corresponding bispecific molecules. cDNA encoding bispecific molecule A2Z-ld or ZA2-d was cloned into recombinat AAV viruses. 293 cells were transduced with recombinant AAV viruses carrying A2Z-Ld or ZA2-d cDNA for 72 hours. Culture medium were collected and 20 μl of supernatant were loaded on SDA PAGE and Western Blot were performed.

### Example 2: Trispecific Antagonists Containing VEGF Binding Domains, Tie2 Pathway Inhibitors and PD-1 Binding Domains

Trispecific constructs GS01-T and GS01-A were constructed. GS01-T contains an PD-1 binding domain (derived from anti-PD-1 mab 2P17), an VEGF binding domain (scFv derived from anti-VEGF antibody As) and an Ang1/Ang2 bining domain (Trebananib peptide) (FIG. 23). GS01-A contains an PD-1 binding domain (derived from anti-PD-1 mab 2P17), an VEGF binding domain (scFv derived from anti-VEGF antibody As) and an Ang2 bining domain (Ang2 binding peptide A2) (FIG. 24).

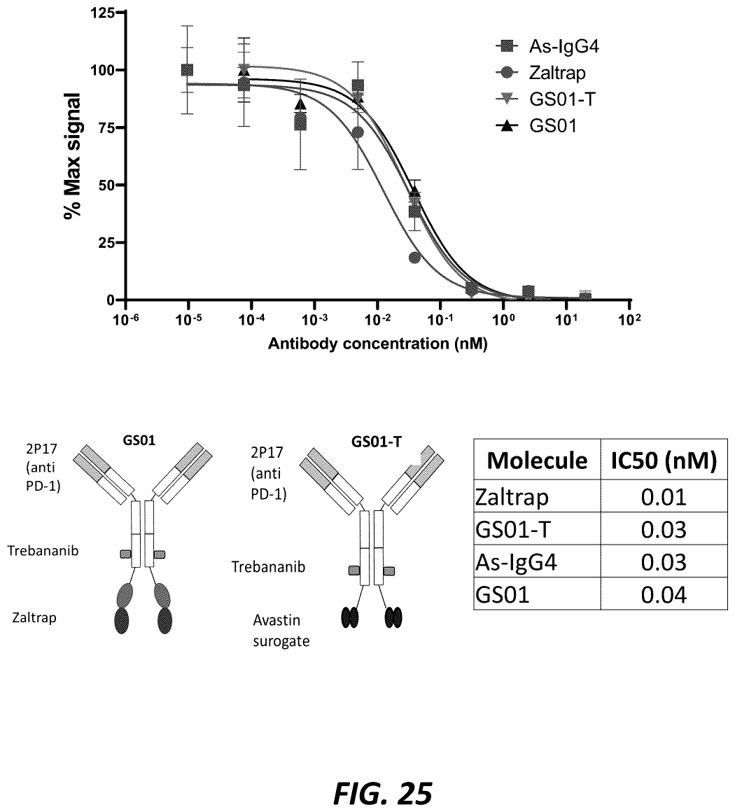

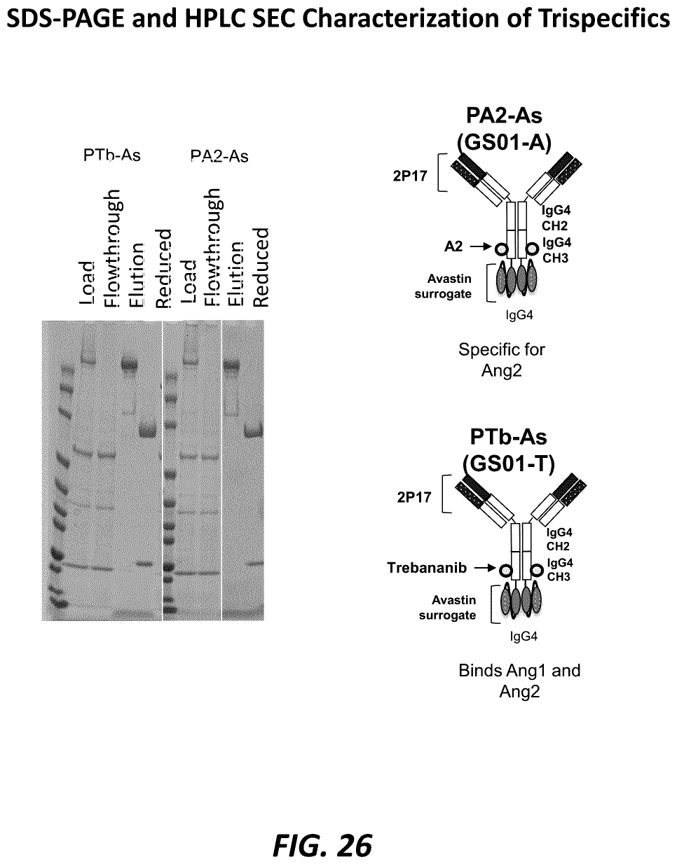

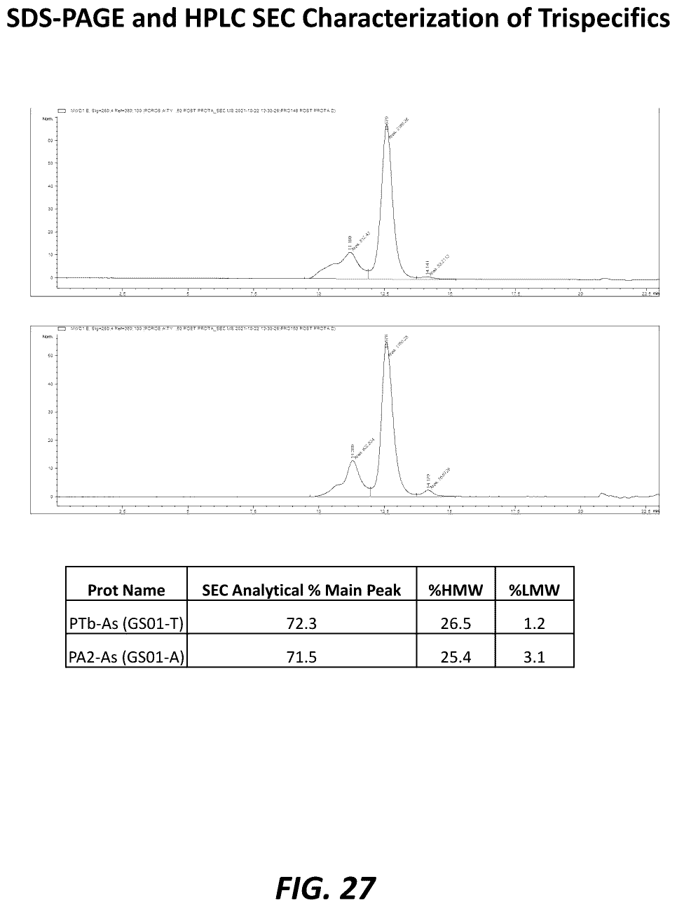

The trispecific contructs were expressed in HEK293 cells and purified with Protein A. FIG. 26 shows SDS-PAGE characterization of trispecific constructs GS01-A and GS01-T. FIG. 27 shows HPLC SEC characterization of trispecific constructs GS01-A and GS01-T. As shown in FIG. 25, both trispecific constructs inhibit VEGF signal in cell-based assay.

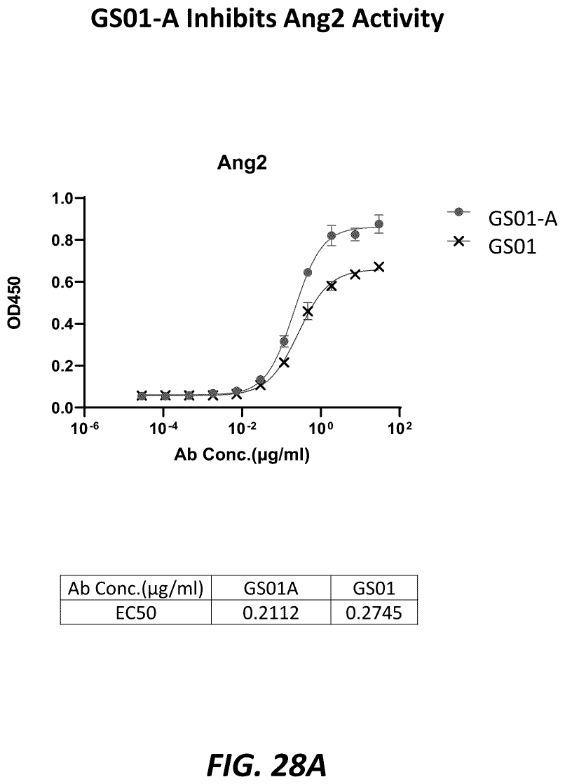

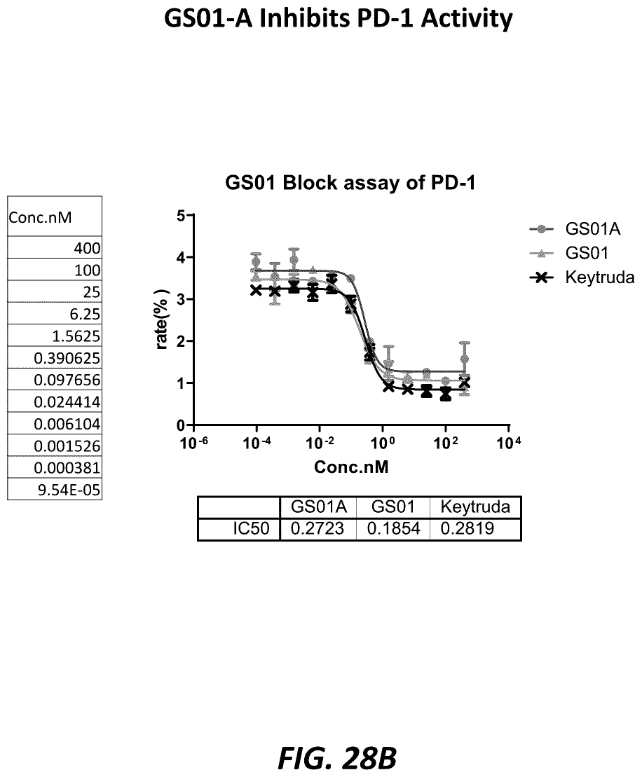

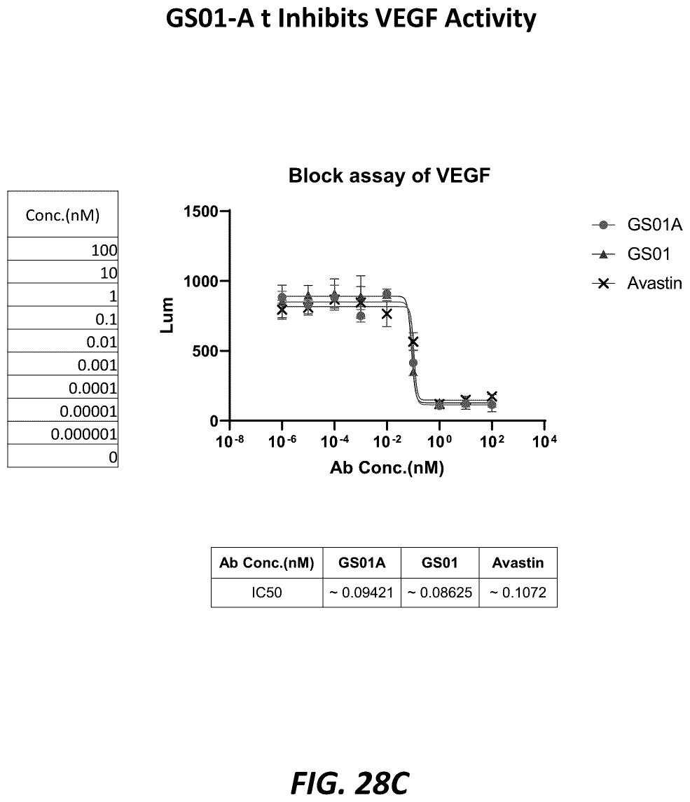

FIGS. 28A-28C show the blocking activity of GS01-A for Ang (FIG. 28A) PD-1 (FIG. 28B) and VEGF (FIG. 28C). GS01-A, which contains a scFv derived from anti-VEGF antibody As as the VEGF binding domain and the Ang2-binding peptide A2 (L1-7) as the Ang2 binding domain, showed higher Ang2 inhibition activity (FIG. 28A) compared to that of GS01 (ZPT-3L), which contains Avastin as the VEGF binding domain and trebananib as the Ang2 binding domain.

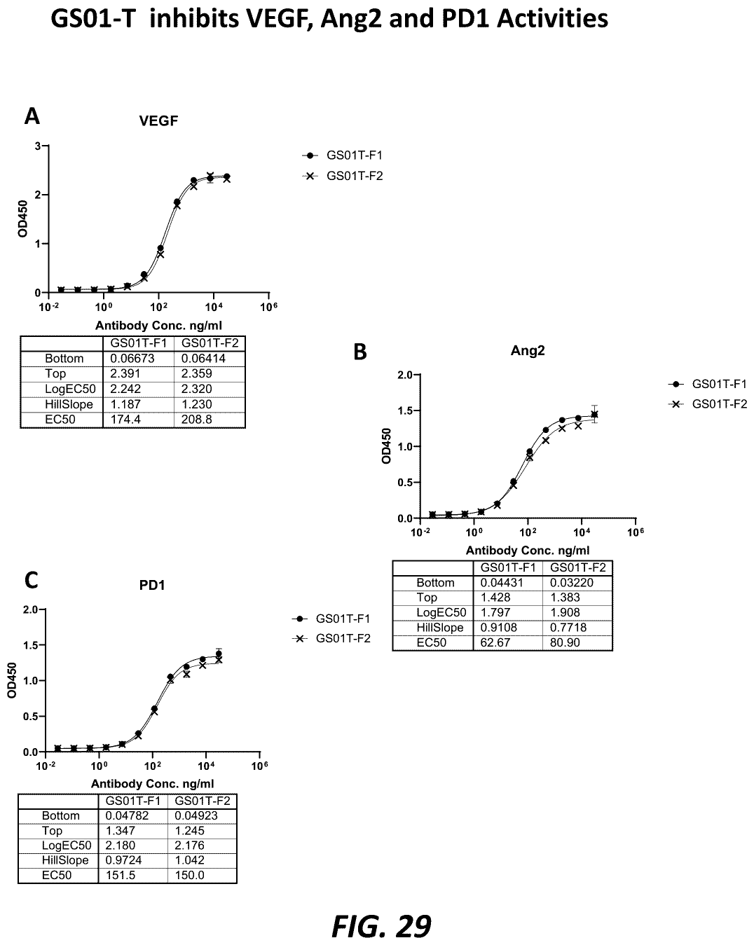

FIG. 29 shows the blocking activity of GS01-T for VEGF (panel A), Ang2 (panel B) and PD-1 (panel C).

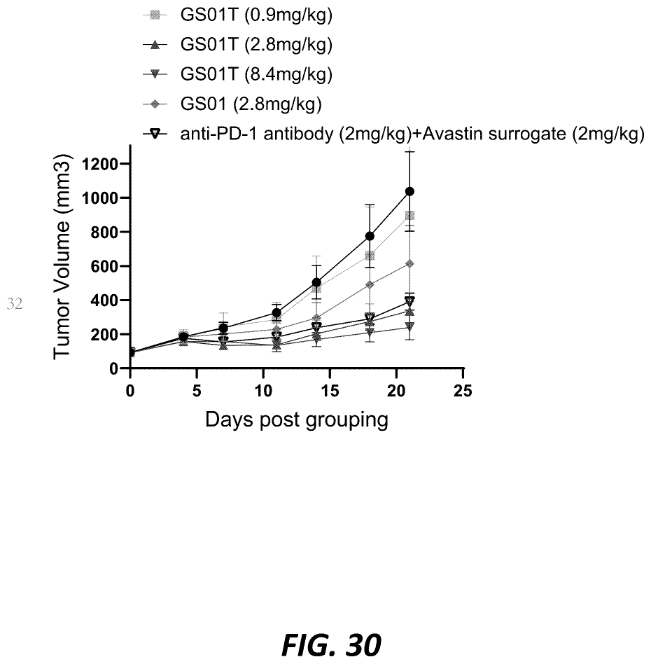

FIG. 30 shows the tumor inhibiting effect of GS01-T in hPD-1 knockin C57BL/6 mice bearing MC38. Briefly, hPD-1/hTIGIT knockin C57BL/6 were subcutaneously injected with MC38 tumor cells for tumor development. Tumor-bearing animals with mean tumor size of 92 mm³ were randomly enrolled into 6 groups consisting of eight each. The five groups were G1 control group (0.9% sodium chloride injection), G2 GS01-T (0.9 mg/kg), G3 GS01-T (2.8 mg/kg), G4 GS01-T (8.4 mg/kg), G5 GS01 (2.8 mg/kg) and G6 Anti-PD-1 antibody+Avastin surrogate (2 mg/kg+2 mg/kg). All test articles were intraperitoneally administrated to tumor-bearing mice twice weekly for total 6 times. The tumor volume growth inhibition rate (TGITV) was calculated. Compared with control group, by day 21 post grouping, the mean tumor volume of the control group was 1037±233 mm³, and that of the G2, G3, G4, G5 and G6 groups was 896±451 mm³, 336±70 mm³, 239±72 mm³, 614±225 mm³ and 390±49 mm³ respectively. The corresponding TGITV for G2˜G6 was 14.9%, 74.2%, 84.5%, 44.7% and 68.4%.

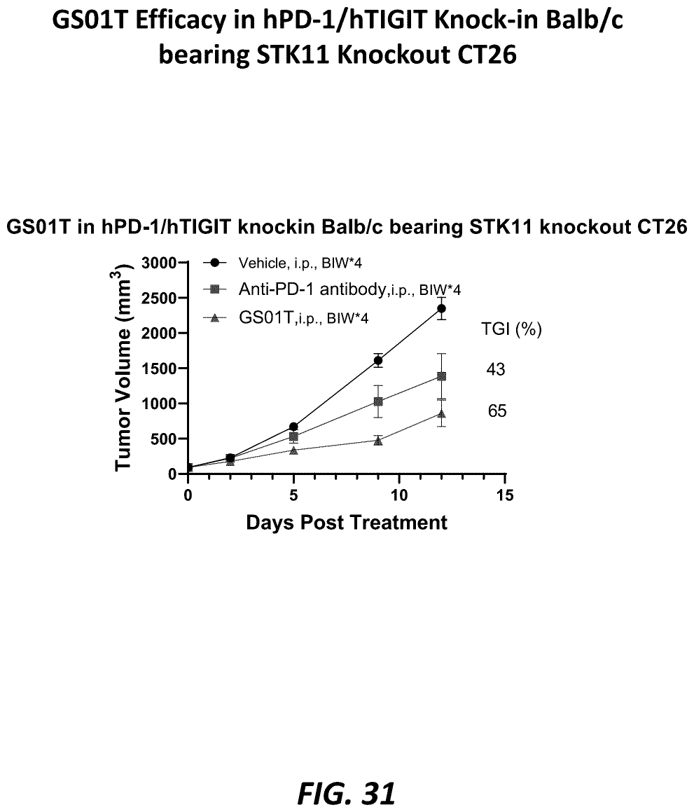

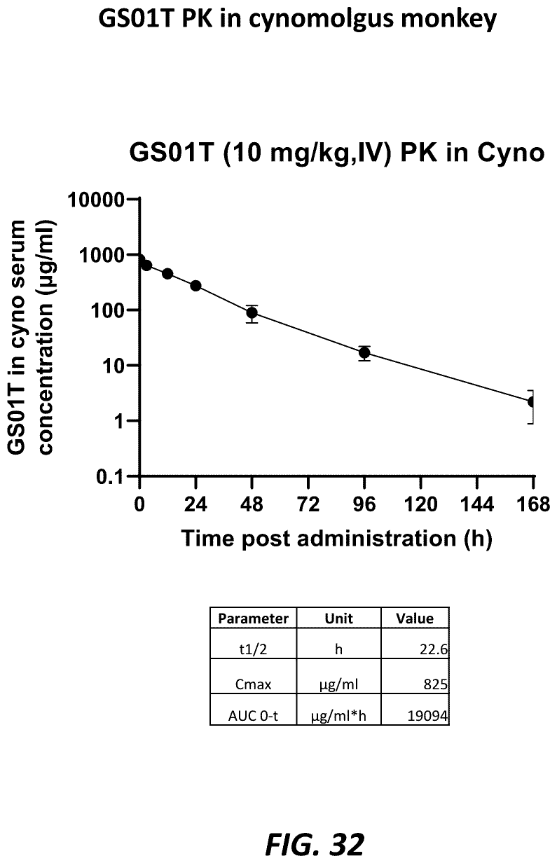

FIG. 31 shows the tumor inhibiting effect of GS01-T in hPD-1/hTIGIT Balb/c mice bearing STK11 KO CT26. Briefly, hPD-1/hTIGIT Balb/c were subcutaneously injected with STK11 knockout CT26 tumor cells for tumor development. Tumor-bearing animals with mean tumor size of 92 mm³ were randomly enrolled into 3 groups consisting of eight each. The three groups were G1 control group (0.9% sodium chloride injection), G2 Anti-PD-1 antibody (10 mg/kg) and G3 GS01T (12.5 mg/kg). All test articles were intraperitoneally administrated to tumor-bearing mice twice weekly for total 4 times. The tumor volume growth inhibition rate (TGI_{TV}) was calculated. Compared with control group, By day 12 post grouping, the mean tumor volume of the control group was 2348±159 mm³, and that of the G2 and G3 groups was 1385±321 mm³ and 857±189 mm³ respectively. The corresponding TGI_{TV} for G2 and G3 was 43.1% and 65.3%. FIG. 32 shows the pharmacokinetics of GS01-T in cynomolgus monkeys.

### Example 3: Bispecific Antagonists Containing VEGF Binding Domains and Trebananib Peptides

Bispecific antagonists containing VEGF binding domains and trebananib peptides were constructed. The configuration and sequences of bispecific VEGF and Ang2/Ang1 inhibitors Bi-TZ, Bi-ZT0, Bi-T0Z, Bi-AT0 and Bi-AmT0 are shown in FIGS. 33-38. These constructs are suitable for cancer therapy

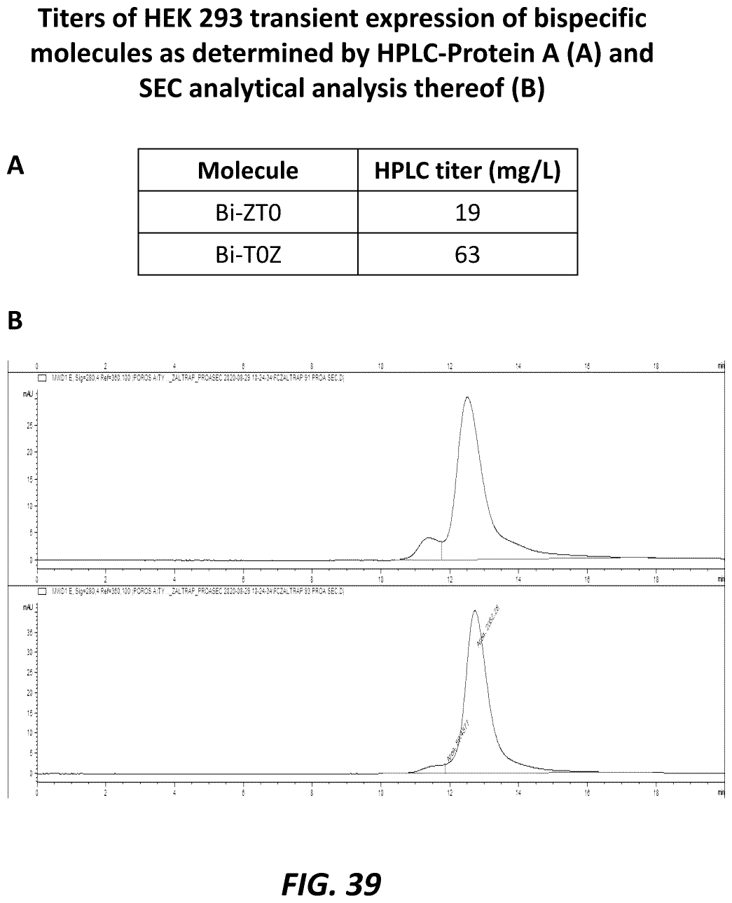

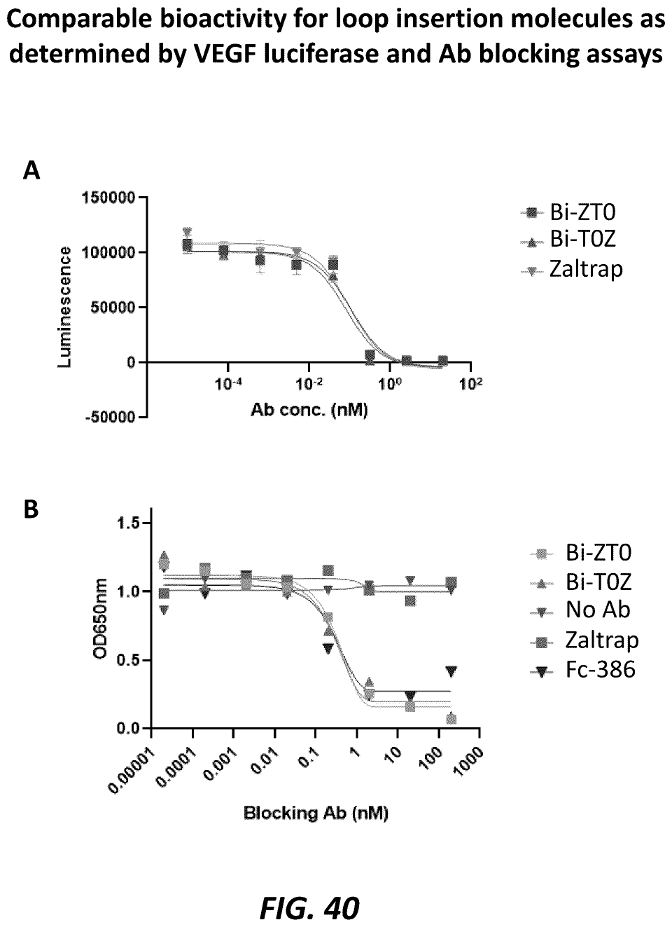

The bispecific contructs were expressed in HEK293 cells and purified with Protein A. FIG. 39 shows titers (Panel A) and SEC characterization of bispecific molecules Bi-ZT0 and Bi-T0Z (panel B). FIG. 40 shows the blocking activity of Bi-ZT0 and Bi-T0Z for VEGF (panel A) and Ang2 (panel B).

### Example 4: Functional Assays for the Multispecific Antagonists

A VEGF-VEGFR-2 blocking assay was used to evaluate the ability of the multispecific antagonists to block the interaction between VEGF and its receptor, VEGFR-2. An aflibercept fusion protein (Zaltrap) was used as a positive control. Briefly, a VEGFR2/NFAT Reporter—HEK293 Recombinant Cell Line (BPS Bioscience Catalog #: 79387) was plated at a density of 30 k cells per well into a white clear-bottom 96-well microplate. After 24 hours, serial dilutions of the indicated molecules and human VEGF165 were added to the wells. After 4 hours of incubation, 100 μl of ONE-Step™ Luciferase reagent was added, the plates were rocked at room temperature for ˜15 min., and the plates read for the luminescence signal.

A Tie2-Ang2 blocking assay was used to evaluate the ability of the multispecific antagonists to block the interaction between Ang2 and its receptor, the Tie2 tyrosine kinase receptor. 96-well assay plates were coated with 0.5 μg/ml of recombinant human Ang2 (Biolegend) in PBS at 4° C. overnight, then blocked with 1% BSA/PBS for 1 hour at room temperature. Serially diluted antagonists were then added to the plates along with recombinant human Tie2 receptor and incubated for 2 hours at room temperature. The plates were washed with Wash Buffer (0.05% Tween-20 in PBS) and then incubated with biotinylated anti-Tie2 antibody for 1 hour at room temperature. After washing with Wash Buffer, Streptavidin-HRP was added to the plate at 1:200 and incubated for 30 minutes at room temperature. The extent of Tie2 receptor binding was detected by measuring light absorbance at 650 nm after addition of TMB to the plate.

The above description is for the purpose of teaching a person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the claimed components and steps in any sequence which is effective to meet the objectives there intended, unless the context specifically indicates the contrary.

What is claimed is:

1. A bispecific antagonist, comprising:
(1) an immunoglobulin scaffold comprising a CH3 domain;
(2) a first targeting domain comprising one or more VEGF or VEGFR binding domains; and
(3) a second targeting domain comprising one or more Tie2 receptor or Ang binding domains.

2. The bispecific antagonist of claim 1, wherein the second targeting domain is inserted into a loop region of the CH3 domain.

3. The bispecific antagonist of claim 1, wherein the second targeting domain is linked to a N-terminal or a C-terminal of the immunoglobulin scaffold.

4. The bispecific antagonist of any one of claims 1-3, wherein the second targeting domain comprises a single copy or two copies of SEQ ID NO:6.

5. The bispecific antagonist of any one of claims 1-4, wherein the first targeting domain comprises SEQ ID NO:1.

6. The bispecific antagonist of any one of claims 1-4, wherein the first targeting domain comprises SEQ ID NO:88 and SEQ ID NO:89.

7. The bispecific antagonist of claim 1, wherein the first targeting domain comprises SEQ ID NO:1 and is attached to a C-terminal of the immunoglobulin scaffold, and wherein the second targeting domain comprises SEQ ID NO:7 and is inserted into a loop region of the CH3 domain of the immunoglobulin scaffold.

8. The bispecific antagonist of claim 1, wherein the first targeting domain comprises SEQ ID NO:1 and is attached to a N-terminal of the immunoglobulin scaffold, and wherein the second targeting domain comprises SEQ ID NO:7 and is linked to a C-terminal of the immunoglobulin scaffold.

9. A pharmaceutical composition, comprising
the bispecific antagonist of any one of claims 1 to 8; and
a pharmaceutically acceptable carrier.

10. A method for treating a condition in a subject, comprising:
administering to the subject an effective amount of the pharmaceutical composition of claim 9,
wherein the condition is wet AMD or cancer.

11. A trispecific antagonist, comprising:
(1) an immunoglobulin scaffold comprising a CH3 domain;
(2) a first targeting domain comprising one or more VEGF or VEGFR binding domains;
(3) a second targeting domain comprising one or more Tie2 receptor or Ang binding domains; and
(4) a third targeting domain comprising one or more PD-1 binding domains.

12. The trispecific antagonist of claim 11, wherein the second targeting domain is inserted into a loop region of the CH3 domain.

13. The trispecific antagonist of claim 11 or 12, wherein the second targeting domain comprises SEQ ID NO:2.

14. The bispecific antagonist of claim 11 or 12, wherein the second targeting domain comprises a single copy or two copies of SEQ ID NO:6.

15. The trispecific antagonist of any one of claims 11-14, wherein the first targeting domain comprises SEQ ID NO:88 and SEQ ID NO:89.

16. The trispecific antagonist of any one of claims 11-15, wherein the third targeting domain comprises SEQ ID NO:90 and SEQ ID NO:91.

17. The trispecific antagonist of claim 11, wherein the first targeting domain comprises SEQ ID NO:88 and SEQ ID NO:89 and is attached to a C-terminal of the immunoglobulin scaffold, wherein the second targeting domain comprises SEQ ID NO:2 and is inserted into a loop region of the CH3 domain of the immunoglobulin scaffold, and wherein the third targeting domain comprises SEQ ID NO:90 and SEQ ID NO:91 and is attached to a N-terminal of the immunoglobulin scaffold.

18. The trispecific antagonist of claim 11, wherein the first targeting domain comprises SEQ ID NO:88 and SEQ ID NO:89 and is attached to a C-terminal of the immunoglobulin scaffold, wherein the second targeting domain comprises SEQ ID NO:7 and is inserted into a loop region of the CH3 domain of the immunoglobulin scaffold, and wherein the third targeting domain comprises SEQ ID NO:90 and SEQ ID NO:91 and is attached to a N-terminal of the immunoglobulin scaffold.

19. A pharmaceutical composition, comprising
the bispecific antagonist of any one of claims 11 to 18; and
a pharmaceutically acceptable carrier.

20. A method for treating a condition in a subject, comprising:
administering to the subject an effective amount of the pharmaceutical composition of claim 19,
wherein the condition is wet AMD or cancer.


SEQUENCE LISTING (124 sequences)

SEQ ID NO: 1 - protein, 205 aa, Artificial Sequence
  Synthetic Aflibercept VEGF binding domain (Z)
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 2 - protein, 52 aa, Artificial Sequence
  Synthetic Trebananib (T)
AQQEECEWDPWTCEHMGSGSATGGSGSTASSGSGSATHQEECEWDPWTCEHM

SEQ ID NO: 3 - protein, 123 aa, Artificial Sequence [sequence omitted]

SEQ ID NO: 4 - protein, 123 aa, Artificial Sequence
  Synthetic Modified Bevacizumab HC variable region (Am)
EVQLVQSGGGVVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSS

SEQ ID NO: 5 - protein, 107 aa, Artificial Sequence [sequence omitted]

SEQ ID NO: 6 - protein, 22 aa, Artificial Sequence
  Synthetic A2 peptide
TNFMPMDDLEQRLYEQFILQQG

SEQ ID NO: 7 - protein, 65 aa, Artificial Sequence
  Synthetic A2d (2 copies of A2)
AQTNFMPMDDLEQRLYEQFILQQGGGGSGGGGSGGGGSGGGGSTNFMPMDDLEQRLYEQFILQQG

SEQ ID NO: 8 - protein, 510 aa, Artificial Sequence
  Synthetic A2Z-d
AQTNFMPMDDLEQRLYEQFILQQGGGGSGGGGSGGGGSGGGGSTNFMPMDDLEQRLYEQFILQQGGGESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGGAQSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKLE

SEQ ID NO: 9 - protein, 512 aa, Artificial Sequence
  Synthetic A2Z-ld
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMGGAQTNFMPMDDLEQRLYEQFILQQGGGGSGGGGSGGGGSGGGGSTNFMPMDDLEQRLYEQFILQQGLETKNQVSLTCLVKGFYPSDIAVEWESNGQPENNYKTTPPVLDSDGSFFLYSRLTVDKSRWQEGNVFSCSVMHEALHNHYTQKSLSLSLGKGGGGGAQSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKLE

SEQ ID NO: 10 - protein, 465 aa, Artificial Sequence
  Synthetic ZA2-s
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGGAQTNFMPMDDLEQRLYEQFILQQGLE

SEQ ID NO: 11 - protein, 506 aa, Artificial Sequence
  Synthetic ZA2-d
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGGAQTNFMPMDDLEQRLYEQFILQQGGGGSGGGGSGGGGSGGGGSTNFMPMDDLEQRLYEQFILQQGLE

SEQ ID NO: 12 - protein, 462 aa, Artificial Sequence
  Synthetic ZA2-ls
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMGGAQTNFMPMDDLEQRLYEQFILQQGLETKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 503 aa, Artificial Sequence
  Synthetic ZA2-ld-1
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMGGAQTNFMPMDDLEQRLYEQFILQQGGGGSGGGGSGGGGSGGGGSTNFMPMDDLEQRLYEQFILQQGLETKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 519 aa, Artificial Sequence
  Synthetic AsA2d heavy chain with L1-7
EVQLVESGGGLVQPGGSLRLSCAASGFTISDYWIHWVRQAPGKGLEWVAGITPAGGYTYYADSVKGRFTISADTSKNTAYLQMNSLRAEDTAVYYCARFVFFLPYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGGAQTNFMPMDDLEQRLYEQFILQQGGGGSGGGGSGGGGSGGGGSTNFMPMDDLEQRLYEQFILQQGLE

SEQ ID NO: 15 - protein, 214 aa, Artificial Sequence
  Synthetic AsA2d light chain
DIQMTQSPSSLSASVGDRVTITCRASQDVSTAVAWYQQKPGKAPKLLIYSASFLYSGVPSRFSGSGSGTDFTLTISSLQPEDFATYYCQQGYGNPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 516 aa, Artificial Sequence
  Synthetic AsA2-ld heavy chain with L1-7
EVQLVESGGGLVQPGGSLRLSCAASGFTISDYWIHWVRQAPGKGLEWVAGITPAGGYTYYADSVKGRFTISADTSKNTAYLQMNSLRAEDTAVYYCARFVFFLPYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMGGAQTNFMPMDDLEQRLYEQFILQQGGGGSGGGGSGGGGSGGGGSTNFMPMDDLEQRLYEQFILQQGLETKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 214 aa, Artificial Sequence; identical to SEQ ID NO: 15
  Synthetic AsA2-ld light chain

SEQ ID NO: 18 - protein, 20 aa, Artificial Sequence
  Synthetic ang2-specific peptide
PIRQEECDWDPWTCEHMWEV

SEQ ID NO: 19 - protein, 20 aa, Artificial Sequence
  Synthetic ang2-specific peptide
TNIQEECEWDPWTCDHMPGK

SEQ ID NO: 20 - protein, 20 aa, Artificial Sequence
  Synthetic ang2-specific peptide
WYEQDACEWDPWTCEHMAEV

SEQ ID NO: 21 - protein, 20 aa, Artificial Sequence
  Synthetic ang2-specific peptide
NRLQEVCEWDPWTCEHMENV

SEQ ID NO: 22 - protein, 20 aa, Artificial Sequence
  Synthetic ang2-specific peptide
AATQEECEWDPWTCEHMPRS

SEQ ID NO: 23 - protein, 20 aa, Artificial Sequence
  Synthetic ang2-specific peptide
LRHQEGCEWDPWTCEHMFDW

SEQ ID NO: 24 - protein, 20 aa, Artificial Sequence
  Synthetic ang2-specific peptide
VPRQKDCEWDPWTCEHMYVG

SEQ ID NO: 25 - protein, 20 aa, Artificial Sequence
  Synthetic ang2-specific peptide
SISHEECEWDPWTCEHMQVG

SEQ ID NO: 26 - protein, 20 aa, Artificial Sequence
  Synthetic ang2-specific peptide
WAAQEECEWDPWTCEHMGRM

SEQ ID NO: 27 - protein, 20 aa, Artificial Sequence
  Synthetic ang2-specific peptide
TWPQDKCEWDPWTCEHMGST

SEQ ID NO: 28 - protein, 20 aa, Artificial Sequence
  Synthetic ang2-specific peptide
GHSQEECGWDPWTCEHMGTS

SEQ ID NO: 29 - protein, 20 aa, Artificial Sequence
  Synthetic ang2-specific peptide
QHWQEECEWDPWTCDHMPSK

SEQ ID NO: 30 - protein, 20 aa, Artificial Sequence
  Synthetic ang2-specific peptide
NVRQEKCEWDPWTCEHMPVR

SEQ ID NO: 31 - protein, 20 aa, Artificial Sequence
  Synthetic ang2-specific peptide
KSGQVECNWDPWTCEHMPRN

SEQ ID NO: 32 - protein, 20 aa, Artificial Sequence
  Synthetic ang2-specific peptide
VKTQEHCDWDPWTCEHMREW

SEQ ID NO: 33 - protein, 20 aa, Artificial Sequence
  Synthetic ang2-specific peptide
AWGQEGCDWDPWTCEHMLPM

SEQ ID NO: 34 - protein, 20 aa, Artificial Sequence
  Synthetic ang2-specific peptide
PVNQEDCEWDPWTCEHMPPM

SEQ ID NO: 35 - protein, 20 aa, Artificial Sequence
  Synthetic ang2-specific peptide
RAPQEDCEWDPWTCAHMDIK

SEQ ID NO: 36 - protein, 20 aa, Artificial Sequence
  Synthetic ang2-specific peptide
HGQNMECEWDPWTCEHMFRY

SEQ ID NO: 37 - protein, 20 aa, Artificial Sequence
  Synthetic ang2-specific peptide
PRLQEECVWDPWTCEHMPLR

SEQ ID NO: 38 - protein, 20 aa, Artificial Sequence
  Synthetic ang2-specific peptide
RTTQEKCEWDPWTCEHMESQ

SEQ ID NO: 39 - protein, 20 aa, Artificial Sequence
  Synthetic ang2-specific peptide
QTSQEDCVWDPWTCDHMVSS

SEQ ID NO: 40 - protein, 20 aa, Artificial Sequence
  Synthetic ang2-specific peptide
QVIGRPCEWDPWTCEHLEGL

SEQ ID NO: 41 - protein, 20 aa, Artificial Sequence
  Synthetic ang2-specific peptide
WAQQEECAWDPWTCDHMVGL

SEQ ID NO: 42 - protein, 20 aa, Artificial Sequence
  Synthetic ang2-specific peptide
LPGQEDCEWDPWTCEHMVRS

SEQ ID NO: 43 - protein, 20 aa, Artificial Sequence
  Synthetic ang2-specific peptide
PMNQVECDWDPWTCEHMPRS

SEQ ID NO: 44 - protein, 20 aa, Artificial Sequence
  Synthetic ang2-specific peptide
FGWSHGCEWDPWTCEHMGST

SEQ ID NO: 45 - protein, 20 aa, Artificial Sequence
  Synthetic ang2-specific peptide
KSTQDDCDWDPWTCEHMVGP

SEQ ID NO: 46 - protein, 20 aa, Artificial Sequence
  Synthetic ang2-specific peptide
GPRISTCQWDPWTCEHMDQL

SEQ ID NO: 47 - protein, 20 aa, Artificial Sequence
  Synthetic ang2-specific peptide
STIGDMCEWDPWTCAHMQVD

SEQ ID NO: 48 - protein, 20 aa, Artificial Sequence
  Synthetic ang2-specific peptide
VLGGQGCEWDPWTCRLLQGW

SEQ ID NO: 49 - protein, 20 aa, Artificial Sequence
  Synthetic ang2-specific peptide
VLGGQGCQWDPWTCSHLEDG

SEQ ID NO: 50 - protein, 20 aa, Artificial Sequence
  Synthetic ang2-specific peptide
TTIGSMCEWDPWTCAHMQGG

SEQ ID NO: 51 - protein, 22 aa, Artificial Sequence
  Synthetic ang2-specific peptide
TNYKPLDELDATLYEHWILQHS

SEQ ID NO: 52 - protein, 22 aa, Artificial Sequence
  Synthetic ang2-specific peptide
QKYQPLDELDKTLYDQFMLQQG

SEQ ID NO: 53 - protein, 22 aa, Artificial Sequence
  Synthetic ang2-specific peptide
LNFTPLDELEQTLYEQWTLQQS

SEQ ID NO: 54 - protein, 22 aa, Artificial Sequence
  Synthetic ang2-specific peptide
QKFQPLDELEQTLYEQFMLQQA

SEQ ID NO: 55 - protein, 22 aa, Artificial Sequence
  Synthetic ang2-specific peptide
QEYEPLDELDETLYNQWMFHQR

SEQ ID NO: 56 - protein, 22 aa, Artificial Sequence
  Synthetic ang2-specific peptide
VKYKPLDELDEILYEQQTFQER

SEQ ID NO: 57 - protein, 22 aa, Artificial Sequence
  Synthetic ang2-specific peptide
TKFQPLDELDQTLYEQWTLQQR

SEQ ID NO: 58 - protein, 22 aa, Artificial Sequence
  Synthetic ang2-specific peptide
TNFQPLDELDQTLYEQWTLQQR

SEQ ID NO: 59 - protein, 22 aa, Artificial Sequence
  Synthetic ang2-specific peptide
QNFKPMDELEDTLYKQFLFQHS

SEQ ID NO: 60 - protein, 22 aa, Artificial Sequence
  Synthetic ang2-specific peptide
VKYKPLDELDEWLYHQFTLHHQ

SEQ ID NO: 61 - protein, 22 aa, Artificial Sequence
  Synthetic ang2-specific peptide
YKFTPLDDLEQTLYEQWTLQHV

SEQ ID NO: 62 - protein, 22 aa, Artificial Sequence
  Synthetic ang2-specific peptide
QNYKPLDELDATLYEHFIFHYT

SEQ ID NO: 63 - protein, 22 aa, Artificial Sequence
  Synthetic ang2-specific peptide
VKFKPLDALEQTLYEHWMFQQA

SEQ ID NO: 64 - protein, 22 aa, Artificial Sequence
  Synthetic ang2-specific peptide
EDYMPLDALDAQLYEQFILLHG

SEQ ID NO: 65 - protein, 22 aa, Artificial Sequence
  Synthetic ang2-specific peptide
YKFNPMDELEQTLYEEFLFQHA

SEQ ID NO: 66 - protein, 22 aa, Artificial Sequence
  Synthetic ang2-specific peptide
SNFMPLDELEQTLYEQFMLQHQ

SEQ ID NO: 67 - protein, 22 aa, Artificial Sequence
  Synthetic ang2-specific peptide
QKFQPLDELEETLYKQWTLQQR

SEQ ID NO: 68 - protein, 22 aa, Artificial Sequence
  Synthetic ang2-specific peptide
QKFMPLDELDEILYEQFMFQQS

SEQ ID NO: 69 - protein, 22 aa, Artificial Sequence
  Synthetic ang2-specific peptide
TKFNPLDELEQTLYEQWTLQHQ

SEQ ID NO: 70 - protein, 22 aa, Artificial Sequence
  Synthetic ang2-specific peptide
HTFQPLDELEETLYYQWLYDQL

SEQ ID NO: 71 - protein, 22 aa, Artificial Sequence
  Synthetic ang2-specific peptide
QKFKPLDELEQTLYEQWTLQQR

SEQ ID NO: 72 - protein, 22 aa, Artificial Sequence
  Synthetic ang2-specific peptide
QTFQPLDDLEEYLYEQWIRRYH

SEQ ID NO: 73 - protein, 22 aa, Artificial Sequence
  Synthetic ang2-specific peptide
SKFKPLDELEQTLYEQWTLQHA

SEQ ID NO: 74 - protein, 720 aa, Artificial Sequence
  Synthetic ZPT-3L(2P17) HC
QVQLVQSGAEVKKPGASVKVSCKASGYTFTSYYIHWVRQAPGQGLEWMGWIFPGSGNSKYNENFKGRVTLTADTSTSTVYMELSSLRSEDTAVYYCASETYDYGDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMGGAQQEECEWDPWTCEHMGSGSATGGSGSTASSGSGSATHQEECEWDPWTCEHMGGTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 75 - protein, 682 aa, Artificial Sequence
  Synthetic ZPT-3S(2P17) HC
QVQLVQSGAEVKKPGASVKVSCKASGYTFTSYYIHWVRQAPGQGLEWMGWIFPGSGNSKYNENFKGRVTLTADTSTSTVYMELSSLRSEDTAVYYCASETYDYGDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMGGQEECEWDPWTCEHMGGTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 76 - protein, 214 aa, Artificial Sequence
  Synthetic ZPT-3S(2P17) LC and ZPT-3L(2P17) LC
DIQMTQSPSFLSASVGDRVTITCKASQNVGTNVAWYQQKPGKAPKALIYSASYRYSGVPSRFSGSGSGTEFTLTISSLQPEDFATYYCQQYYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 767 aa, Artificial Sequence
  Synthetic Tri-PT0As (GS01T)HC
QVQLVQSGAEVKKPGASVKVSCKASGYTFTSYYIHWVRQAPGQGLEWMGWIFPGSGNSKYNENFKGRVTLTADTSTSTVYMELSSLRSEDTAVYYCASETYDYGDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMGGAQQEECEWDPWTCEHMGSGSATGGSGSTASSGSGSATHQEECEWDPWTCEHMGGTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSEVQLVESGGGLVQPGGSLRLSCAASGFTISDYWIHWVRQAPGKGLEWVAGITPAGGYTYYADSVKGRFTISADTSKNTAYLQMNSLRAEDTAVYYCARFVFFLPYAMDYWGQGTLVTVSSGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQGYGNPFTFGQGTKVEIK

SEQ ID NO: 78 - protein, 214 aa, Artificial Sequence; identical to SEQ ID NO: 76
  Synthetic Tri-PT0As (GS01T)LC

SEQ ID NO: 79 - protein, 780 aa, Artificial Sequence
  Synthetic Tri-PA20As (GS01-A) HC
QVQLVQSGAEVKKPGASVKVSCKASGYTFTSYYIHWVRQAPGQGLEWMGWIFPGSGNSKYNENFKGRVTLTADTSTSTVYMELSSLRSEDTAVYYCASETYDYGDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMGGAQTNFMPMDDLEQRLYEQFILQQGGGGSGGGGSGGGGSGGGGSTNFMPMDDLEQRLYEQFILQQGLETKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSEVQLVESGGGLVQPGGSLRLSCAASGFTISDYWIHWVRQAPGKGLEWVAGITPAGGYTYYADSVKGRFTISADTSKNTAYLQMNSLRAEDTAVYYCARFVFFLPYAMDYWGQGTLVTVSSGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQGYGNPFTFGQGTKVEIK

SEQ ID NO: 80 - protein, 506 aa, Artificial Sequence
  Synthetic Bi-TZ
AQQEECEWDPWTCEHMGSGSATGGSGSTASSGSGSATHQEECEWDPWTCEHMGGGGGESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 81 - protein, 490 aa, Artificial Sequence
  Synthetic Bi0ZT0
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMGGAQQEECEWDPWTCEHMGSGSATGGSGSTASSGSGSATHQEECEWDPWTCEHMGGTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 82 - protein, 505 aa, Artificial Sequence
  Synthetic Bi-T0Z
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMGGAQQEECEWDPWTCEHMGSGSATGGSGSTASSGSGSATHQEECEWDPWTCEHMGGTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 83 - protein, 509 aa, Artificial Sequence
  Synthetic Bi-AT0 HC
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMGGAQQEECEWDPWTCEHMGSGSATGGSGSTASSGSGSATHQEECEWDPWTCEHMGGTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 214 aa, Artificial Sequence
  Synthetic Bi-AT0 LC
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 509 aa, Artificial Sequence
  Synthetic Bi-AmT0 HC
EVQLVQSGGGVVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMGGAQQEECEWDPWTCEHMGSGSATGGSGSTASSGSGSATHQEECEWDPWTCEHMGGTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 214 aa, Artificial Sequence; identical to SEQ ID NO: 84
  Synthetic Bi-AmT0 LC

SEQ ID NO: 87 - protein, 205 aa, Artificial Sequence
  Synthetic afilibercept (aglycosylated)
SDTGRPFVEMYSEIPEIIHMTEGRELVIPCRVTSPEITVTLKKFPLDTLIPDGKRIIWDSRKGFIISEATYKEIGLLTCEATVNGHLYKTNYLTHRQTNTIIDVVLSPSHGIELSVGEKLVLECTARTELNVGIDFNWEYPSSKHQHKKLVNRDLKTQSGSEMKKFLSTLTIDGVTRSDQGLYTCAASSGLMTKKESTFVRVHEK

SEQ ID NO: 88 - protein, 120 aa, Artificial Sequence
  Synthetic As HCVR
EVQLVESGGGLVQPGGSLRLSCAASGFTISDYWIHWVRQAPGKGLEWVAGITPAGGYTYYADSVKGRFTISADTSKNTAYLQMNSLRAEDTAVYYCARFVFFLPYAMDYWGQGTLVTVSS

SEQ ID NO: 89 - protein, 107 aa, Artificial Sequence
  Synthetic As LCVR
DIQMTQSPSSLSASVGDRVTITCRASQDVSTAVAWYQQKPGKAPKLLIYSASFLYSGVPSRFSGSGSGTDFTLTISSLQPEDFATYYCQQGYGNPFTFGQGTKVEIK

SEQ ID NO: 90 - protein, 117 aa, Artificial Sequence
  Synthetic 2P17 HCVR
QVQLVQSGAEVKKPGASVKVSCKASGYTFTSYYIHWVRQAPGQGLEWMGWIFPGSGNSKYNENFKGRVTLTADTSTSTVYMELSSLRSEDTAVYYCASETYDYGDYWGQGTLVTVSS

SEQ ID NO: 91 - protein, 107 aa, Artificial Sequence
  Synthetic 2P17 LCVR
DIQMTQSPSFLSASVGDRVTITCKASQNVGTNVAWYQQKPGKAPKALIYSASYRYSGVPSRFSGSGSGTEFTLTISSLQPEDFATYYCQQYYSYPYTFGQGTKLEIK

SEQ ID NO: 92 - protein, 330 aa, Artificial Sequence [sequence omitted]

SEQ ID NO: 93 - protein, 330 aa, Artificial Sequence
  Synthetic IgG1 CH1-CH2-CH3 with a K447A mutation
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 94 - protein, 330 aa, Artificial Sequence
  Synthetic IgG1 CH1-CH2-CH3 with the N297A mutation
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 326 aa, Artificial Sequence
  Synthetic wild type IgG2 CH1-CH2-CH3
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 96 - protein, 325 aa, Artificial Sequence
  Synthetic IgG2 CH1-CH2-CH3 with carboxy terminal lysine deleted
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 97 - protein, 327 aa, Artificial Sequence
  Synthetic Wild type IgG4 CH1-CH2-CH3
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 98 - protein, 327 aa, Artificial Sequence
  Synthetic IgG4 CH1-CH2-CH3 with the hinge S231P mutation
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLSLGK

SEQ ID NO: 99 - protein, 327 aa, Artificial Sequence
  Synthetic IgG4 CH1-CH2-CH3 with S231P and K447A
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLSLGA

SEQ ID NO: 100 - protein, 326 aa, Artificial Sequence
  Synthetic IgG4 CH1-CH2-CH3 with S231P and the carboxy-terminal lysine deleted
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLSLG

SEQ ID NO: 101 - protein, 107 aa, Artificial Sequence [sequence omitted]

SEQ ID NO: 102 - protein, 227 aa, Artificial Sequence
  Synthetic wild type IgG1 Fc
DKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 227 aa, Artificial Sequence
  Synthetic a-glycosylated IgG1 Fc
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 225 aa, Artificial Sequence
  Synthetic wild type IgG4 Fc
GPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 105 - protein, 225 aa, Artificial Sequence
  Synthetic IgG4 Fc with hinge mutation
GPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLSLGK

SEQ ID NO: 106 - protein, 232 aa, Artificial Sequence
  Synthetic wild type IgG2 Fc
DKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 107 - protein, 226 aa, Artificial Sequence
  Synthetic IgG1 Fc with deletion of carboxy- terminal lysine
DKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 108 - protein, 226 aa, Artificial Sequence
  Synthetic a-glycosylated IgG1 Fc with deletion of carboxy-terminal lysine
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 109 - protein, 224 aa, Artificial Sequence
  Synthetic IgG4 Fc with hinge mutation and deletion of carboxy-terminal lysine
GPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLSLG

SEQ ID NO: 110 - protein, 231 aa, Artificial Sequence
  Synthetic IgG2 Fc with deletion of carboxy- terminal lysine
DKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 111 - protein, 103 aa, Artificial Sequence
  Synthetic IgG1 CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 112 - protein, 102 aa, Artificial Sequence
  Synthetic IgG4 CH1
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKY

SEQ ID NO: 113 - protein, 3 aa, Artificial Sequence
  Synthetic Linker sequence
GGG

SEQ ID NO: 114 - protein, 5 aa, Artificial Sequence
  Synthetic Linker sequence
GGGGS

SEQ ID NO: 115 - protein, 10 aa, Artificial Sequence
  Synthetic Linker sequence
GGGGSGGGGS

SEQ ID NO: 116 - protein, 15 aa, Artificial Sequence
  Synthetic Linker sequence
GGGGSGGGGSGGGGS

SEQ ID NO: 117 - protein, 20 aa, Artificial Sequence
  Synthetic Linker sequence
GGGGSGGGGSGGGGSGGGGS

SEQ ID NO: 118 - protein, 30 aa, Artificial Sequence
  Synthetic Linker sequence
GGGGSGGGGSGGGGSGGGGSGGGGSGGGGS

SEQ ID NO: 119 - protein, 12 aa, Artificial Sequence
  Synthetic amino acid
ETFLSTNKLENQ

SEQ ID NO: 120 - protein, 13 aa, Artificial Sequence
  Synthetic amino acid
NSLSNASEFRAPY

SEQ ID NO: 121 - protein, 7 aa, Artificial Sequence
  Synthetic amino acid
NLLMAAS

SEQ ID NO: 122 - protein, 123 aa, Artificial Sequence [sequence omitted]

SEQ ID NO: 123 - protein, 123 aa, Artificial Sequence; identical to SEQ ID NO: 4
  Synthetic Improved bevacizumab - HCVR

SEQ ID NO: 124 - protein, 107 aa, Artificial Sequence [sequence omitted]